(19) United States
(12) Patent Application Publication
(10) Pub. No.: US 2026/0337667 A1
(43) Pub. Date: Oct. 29, 2026

(54) REAGENT FOR IN SITU HYBRIDIZATION QUALITY CONTROL

(71) Applicant: RESOLVE BIOSCIENCES GMBH, Monheim am Rhein (DE)

(72) Inventors: Seiichiro WATANABE, Monheim am Rhein (DE); Lawrence GREENFIELD, Monheim am Rhein (DE); Christian KORFHAGE, Monheim am Rhein (DE); Cheng Frank ZHONG, Monheim am Rhein (DE); Frank REINECKE, Monheim am Rhein (DE)

(73) Assignee: RESOLVE BIOSCIENCES GMBH, Monheim am Rhein (DE)

(21) Appl. No.: 19/491,501

(22) PCT Filed: Jun. 16, 2024
(86) PCT No.: PCT/EP2024/066706

§ 371 Date: Dec. 9, 2025

Related U.S. Application Data
(60) Provisional application No. 63/508,732, filed on Jun. 16, 2023.

(51) Int. Cl. C12Q 1/6841

(52) U.S. Cl. CPC C12Q 1/6841

(57) ABSTRACT

A monitoring probe comprising a nucleotide sequence, which corresponds to a translator (c) allowing the hybridisation of a signal oligonucleotide; an identifier element allowing the hybridisation of a decoding element, wherein the Identifier comprises a sequence which is essentially complementary to a section of the unique set identifier of an identifier element (T) of a corresponding decoder; a target nucleic add sequence allowing the hybridisation of a binding element. Both probe and target may be immobilised. The target may be obtained from a tissue sample. The probe may be used to monitor ambient temperature ranges. The probe supervises the quality (QC) of manufactured reagents and kits in the field of in-situ hybridisation during all stages of manufacturing. Kits and control slides comprising the monitoring probes.

### RELATED APPLICATIONS

The present application is a US regional phase entry of international application no PCT/EP2024/066706, filed Jun. 16, 2024, and claiming priority to U.S. Prov. Ser. No. 63/508,732, filed Jun. 16, 2023, each of which is hereby incorporated by reference in its entirety.

### SEQUENCE LISTING

The instant application contains a Sequence Listing which has been submitted electronically in XML format and is hereby incorporated by reference in its entirety. Said XML copy, created on Dec. 18, 2025, is named Resolve_045US_SL.xml and is 51,586 bytes in size.

### FIELD OF THE DISCLOSURE

In order to supervise the quality of manufactured reagents and kits, especially in the field of laboratory (especially in diagnostic labs) and/or medical applications, an effective quality control (QC) during all stages of manufacturing is essential and a requirement for pharmaceutical approval. A good QC ensures that the produced materials will meet the intended performance when used by a customer.

Especially in the technical field of in situ hybridization, surrogate QC methods (pH, ionic strength, absorbance, orthogonal analytical assays) are used to measure individual kit components. However, these methods do not measure the overall performance of the entire assay or workflow. Failure of assay performance due to interactions between the individual kit components, interaction between the kits with the instrument, user error, laboratory environmental conditions outside the desired assay range or use of unacceptable additional components supplied by the user are missed when controls are used which do not monitor the entire workflow. Especially unforeseen misuse and/or unforeseen interactions are difficult to monitor with QC-test methods which monitor only very limited portions of the assay.

On the other hand, more holistic quality controls for in situ hybridization validation, require carrying out the entire workflow to assess the final performance data of a sample. These controls typically run in parallel to the customer's test with standardized tissue and/or cell line samples.

Although these types of controls can monitor interactions among the individual kit components, environment, and instrument, they give little information as to the source of performance failure. Variability, for example inherent in tissue and cell lines, make it difficult to rule out the sample itself as a source of failed performance. In addition, failure of the entire workflow performance gives little insight as to which part of the workflow caused the failure. Because of the external nature of these types of QC, they need to be run as a separate sample and cannot be used as an internal control.

There is the need for a nonbiological, easy to manufacture, set of reagents that can monitor the entire in situ hybridization workflow and can be designed to identify different critical steps of the process.

The present disclosure provides reagents and methods which overcome these problems.

### BACKGROUND

In the prior art some approaches for QC-assays have been established.

Wilcox, J N (1993). Fundamental Principles of In Situ Hybridization. J of Histochem. Cytochem. 41 (12): 1725-1733 suggest the use of control tissue as an exogenous control. As a negative control, the article recommends the use of an RNA target that is not expressed in the tissue of interest. Alternatively, the use of probes to the antisense sequence can serve as a negative control. As a positive control, the article recommends the use of an RNA target of known distribution and expression level. However, this method has the disadvantage that the use of a gene expressed in some tissue precludes the ability to measure expression of that gene, is qualitative due to not knowing the exact level of expression and cannot be randomly distributed across the sample.

Rosenstraus, M; Wang, Z; Chang, S-Y; DeBonville, D; and Spadaro, J P (1998). An Internal Control for Routine Diagnostic PCR: Design, Properties, and Effect on Clinical Performance. J. Clin. Micro. 36 (1): 191-197, describe the use of internal control for PCR diagnostic tests consisting of a synthetic exogenous RNA spiked into the clinical sample at the initiation of sample preparation, prior to amplification. However, this method has the disadvantage that the internal control described measures all steps required for purification of nucleic acid and amplification of the purified target. Spiking in of an exogenous RNA control does not specifically measure hybridization efficiency.

Buckwalter, S P; Sloan, L M; Cunningham, S A; Espy, M J; Uhl, J R; Jones, M F; Vetter, E A; Mandrekar, J; Cockerill III, F R; Pritt, B S; Patel, R; Wengenack, N L. (2014). Inhibition Controls for Qualitative Real-Time PCR Assays: Are They Necessary for All Specimen Matrices? J. Clin. Micro. 52 (6): 2139-2143 describe the diversity of internal controls used in amplification reactions, such as plasmids, whole viruses, genomic DNA, and whole organisms. It describes the frequency of clinical samples demonstrating inhibition. However, the article only describes the use of controls for monitoring inhibition of amplification.

Zambenedetti, M R; Pavoni, D P; Dallabona, A C; Dominguez, A C; Poersch, CdO; Pragoso, S P; and Krieger, M A (2017). Internal Control for Real-Time Polymerase Chain Reaction Based on MS2 Bacteriophage for RNA Viruses Diagnostics. Mem Inst Oswaldo Cruz, Rio de Janeiro. 112 (5): 339-347 describe that an internal control is necessary to differentiate the true negative results from a failure in the workflow. The article also assesses the importance of using an internal control to assess the sample preparation steps prior to amplification. However, the described internal control measures failure of amplification without differentiating which steps within the process result in failed performance.

Xie, F; Timme, K A; and Wood, J R. (2018). Using Single Molecule mRNA Fluorescent in Situ Hybridization (RNA-FISH) to Quantify mRNAs in Individual Murine Oocytes and Embryos. Sci. Rep. 8 (1): 7930-7941 describe the use of naturally expressed genes as a positive control and specially designed non hybridizing probes as a negative control. Alternative approaches for designing negative controls include running samples omitting some of the probes. These approaches require the use of a second sample. However, the use of an internally expressed gene does not permit absolution quantification of the degree of inhibition since the exact number of target genes are unknown. Omission of a probe set on a second sample requires the processing of a second sample.

Tang, Z; Gu, J; Tang, G; and Medeiros, L J. (2018). Quality Assurance/Quality Control of Fluorescence in Situ Hybridization Tests in Hematologic Malignancies. OBM Genetics. 2 (4): doi:10.21926/obm.genet.1804038 describes the use of quality assurance/quality control (QA/QC) measurements applied for FISH tests by following the American College of Medical Genetics and Genomics (ACMGG) technical standards and guideline. The ACMGG guidelines recommend the use of both an external positive and external negative control for each batch of clinical samples. However, external controls require separate samples to be run, which reduces sample throughput and increases the cost of the test. In addition, the use of an external control does not serve as controls for the assay using the actual sample.

Aralar, A; Yuan, Y; Chen, K; Geng, Y; Velez D O; Sinha, M; Lawrence, S M; and Fraley, S I. (2020) Improving Quantitative Power in Digital PCR through Digital High-Resolution Melting. J. Clin. Micro. 58 (6): e00325-20 describe the use of an exogenous internal control. It defines an internal control (IC) as a nontarget DNA sequence present in each reaction mixture and is coamplified simultaneously with the target sequence. Furthermore, it describes that without an IC, a negative reaction (no signal) can mean that there was no target sequence present in the reaction mixture or that the reaction was inhibited, resulting in a false-negative call. However, as with other amplification-based internal controls, the approach measures all steps required for amplification. It lacks the ability to identify which steps during sample preparation and amplification resulted in the failure/inhibition.

Young, A P; Jackson, D J; and Wyeth, R C. (2022) A Technical Review and Guide to RNA Fluorescence in situ Hybridization. Peer J. March 19; 8:e8806; (Section on Controls for an ISH experiment); describes the use of a positive control against an internally expressed gene. As a negative control, the publication suggests the use of RNase treatment of a negative control sample. The publication suggests an alternative negative control would be to include a probe to the antisense sequence. However, using probes against genes expressed in some tissues precludes the ability to detect expression of that gene in any tissue. In addition, it is a relative internal control in that it is unknown the exact level of expression of the targeted positive control gene. This form of positive control is dependent on where within the tissue the gene is expressed and cannot be distributed randomly across the slide. A separate treatment of a sample is used to include a negative control in a run.

Li, J. (2005). Internal Control for In Situ Hybridization. United States Patent Application 2005/0272032 A1 Publication Date: Dec. 8, 2005 describe a method for monitoring the quality of in situ hybridization analysis of a nuclear DNA target in a tissue or cell sample using a mitochondrial DNA probe as an internal control. The invention also provides a reagent for in situ hybridization detection of a nuclear DNA target and mitochondrial DNA target in a tissue or cell sample. However, the approach uses nontargeted DNA sequences inherent in the sample. The absolute amount of internal control target is unknown, therefore efficiency cannot be determined.

FISH Reference Standard. Horizon. Catalogue No. HD717Certificate of Analysis describes as a quality control, Horizon's Fish Standard uses negative and positive cell line cores and assesses genomic aberrations specific to the FISH assay. However, the use of cell lines to qualify this quality control is dependent on biological variability. In addition, failure to pass the qualification gives no indication as to what step of the entire workflow failed.

Commercially available PeptiQuant™ Plus. instrument performance kits. The product literature describes the use of a panel of peptides spiked into samples as quality controls for mass spectrometry. The approach enables multiplexing, spiking a known quantity into the sample and covers a broad dynamic range of masses. However, the approach is limited to mass spectrometry.

Commercially available RNAscope® reference guide: Guidelines and Protocols for Obtaining Optimized RNA in situ Hybridization Results with Any Tissue Type from Any Species. ACD. This recommends the use of two levels of quality control: A technical workflow check and a sample/RNA quality check. The technical quality control check ensures that the assay and reagents are working correctly. Recommends using a control sample tested on two separate slides: one slide with a housekeeping gene positive control probe and another slide with a nonspecific bacterial gene negative control. They recommend checking tissue sample quality with positive and negative controls before running the experiment. Their recommended positive and negative controls are genes inherently expressed in the sample. However, it requires the use of additional positive and negative samples. Decreases throughput. Increases overall cost of running a sample with control. When using additional control samples, it does not serve as controls measuring characteristic assay of the sample itself.

Against this background, it is an object underlying the present disclosure to provide a method by means of which the disadvantages of the prior art methods can be reduced or even overcome.

### SUMMARY OF THE DISCLOSURE

In a first aspect the disclosure pertains to monitoring probes comprising at least one element having a nucleotide sequence, wherein said element is selected from the group consisting of
a translator (c) element comprising a nucleotide sequence allowing the binding/hybridization of a signal oligonucleotide;
an identifier element t (t) comprising a nucleotide sequence allowing the binding/hybridization of a decoding element;
a target nucleic acid sequence allowing the binding/hybridization of a binding element.

In a second aspect the disclosure pertains to monitoring probes comprising at least one identifier element (t) comprising a nucleotide sequence which is essentially complementary to at least a section of the unique set identifier sequence of an identifier element (T) of a corresponding decoding element. In one embodiment the nucleotide acid sequence of the monitoring probe is attached to a particle selected from a non-magnetic bead particle, magnetic bead particle, a streptavidin coated particle, a biotin coated particle, and/or any combination thereof. In another embodiment the probe further comprises a binding element complementary to at least a section of a target nucleotide acid sequence. In a further embodiment the nucleic acid of the monitoring probe is attached, preferably by a lasting attraction between atoms or ions that enable the formation of a molecule; the bond may result from the electrostatic force between oppositely charged ions as in ionic bonds, or through the sharing of electrons as in covalent bonds. Thus, the attachment is preferably by covalent bond, hydrogen bonds, a van-der-Waals interaction and/or ionic bond to a particle selected from a magnetic bead particle, a streptavidin coated particle, a biotin coated particle, and/or any combination thereof.

In yet a further embodiment the monitoring probe is designed to facilitate a fluorescent signal substantially equivalent to the signal intensity obtained for the target biological analytes. This can be done by a direct labelling of the monitoring probe, however, preferably by interacting with a signal oligonucleotide which either binds directly to the monitoring probe and/or indirectly via one or more decoding element(s). The use of decoding elements between the monitoring probe and the signal oligonucleotide allows for a multiplex approach, e.g., the use of the same signal for set of different monitoring probe(s), and/or the use of different signal oligonucleotides interacting with the same monitoring probe(s), e.g., in order to differentiate between different reaction steps and/or different reaction conditions.

In yet a further embodiment the nucleic acid sequence of the monitoring probe contains a spacer element between the portion to be immobilized onto the particle and the nucleic acid identifier element (t).

In yet a further embodiment the monitoring probe is immobilized on a slide surface through a poly-lysine coating of the slide surface. In yet a further embodiment the monitoring probe is immobilized to the poly-lysine coated slide surface using homobifunctional or heterobifunctional cross-linking reagents via a reactive group. In yet a further embodiment the monitoring probe or the slide surface comprise a reactive group consisting of an alkyne or azide, and the other contact surface comprises the partner azide or alkyne, such that the immobilization can occur using click chemistry.

In yet a further embodiment the size of the monitoring probe is 250 nm or less; 225 nm or less; 210 nm or less; but at least 90 nm, at least 130 nm at least 180 nm, in one embodiment the size of the monitoring probe is about 200 nm.

In a second aspect the disclosure pertains to method to monitor a in situ hybridization multiplex reaction wherein the method comprises at least the steps of:
1. contacting the analyte nucleotide sequence with at least one detecting nucleic acid sequence in order to conduct an in-situ hybridization; and
2. adding the monitoring probe according to any of claims 1 to 9 before, during and/or after adding the detecting nucleic acid sequence in step 1.

In a further embodiment the method to monitor an in situ hybridization multiplex reaction comprises at least one monitoring probe as described herein, wherein the in situ hybridization multiplex reaction is characterized by comprising:
a.) a binding element which is essentially complementary to at least a section of an analyte nucleotide sequence to be detected and an identifier element (T) comprising a nucleotide sequence which is unique to the analyte nucleotide sequence; and/or
b.) a decoding element comprising an identifier element (t) comprising a nucleotide sequence which is essentially complementary to at least a section of the unique set identifier sequence of the identifier element (T) of the corresponding binding element, and a translator element (c) comprising a nucleotide sequence allowing a specific hybridization of a signal oligonucleotide; and/or
c.) a signal oligonucleotide comprising a translator connector element (C) comprising a nucleotide sequence which is essentially complementary to at least a section of the nucleotide sequence of a translator element (c) comprised in a decoding oligonucleotide, and a signal element facilitating a signal which is specific for the polymorphic analyte; and
wherein the method comprises a monitoring probe comprising at least an identifier element (t) comprising a nucleotide sequence which is essentially complementary to at least a section of the unique set identifier sequence of the identifier element (T) of the corresponding binding element; and
wherein the method comprises at least the steps of:
1. contacting the binding element with the analyte nucleotide sequence;
2. contacting the binding element with the decoding element; and
3. contacting the signal oligonucleotide with the decoding element; and
wherein the monitoring probe is added before, during and/or after adding the decoding element in step 2.

The in-situ hybridization multiplex reaction is described in more details in this description, however, it should be mentioned in a first overview, that the monitoring probe of the present invention is designed to allow the monitoring of the correct in situ hybridization multiplex reaction by being part of the reaction, thereby being exposed to the same reactants and reactions steps as used in the in situ hybridization multiplex reaction.

In one embodiment the method/monitoring probe comprises a set of monitoring probes, wherein for each of the different identifier elements (t) of the decoding elements the set of monitoring probes comprises at least one monitoring probe with the same identifier elements (t).

In a third aspect the disclosure pertains to a detection mix comprising the monitoring probe as described herein, and further comprising at least one element selected from a decoy element consisting either of a magnetic bead particle, a streptavidin coated particle, a biotin coated particle, and/or any combination thereof; optionally bound to a nucleic acid oligonucleotide, which does not correspond to the identifier element (t); a decoding element comprising an identifier element (t) comprising a nucleotide sequence which is essentially complementary to at least a section of the unique set identifier sequence of the identifier element (T) of the corresponding binding element, and a translator element (c) comprising a nucleotide sequence allowing a specific hybridization of a signal oligonucleotide; a binding element which is essentially complementary to at least a section of an analyte nucleotide sequence to be detected and an identifier element (T) comprising a nucleotide sequence which is unique to the analyte nucleotide sequence; and a signal oligonucleotide comprising a translator connector element (C) comprising a nucleotide sequence which is essentially complementary to at least a section of the nucleotide sequence of a translator element (c) comprised in a decoding oligonucleotide, and a signal element facilitating a signal which is specific for the polymorphic analyte; as well as any combination thereof.

In a fourth aspect the disclosure encompasses a method, wherein the monitoring probe as disclosed herein, can be detected by a signal element with a fluorophore being the same or being different from the fluorophore used in the detection of the target nucleic acid sequence that permits normalizing the signal of the immobilized monitoring probe. In yet a further embodiment a first signal element with a first fluorophore allows for measurement of hybridization efficiency of the decoder and readout probe labelled with a second signal element with a second fluorophore by measuring the ratio between the first and the second fluorophore. In yet a further embodiment the first signal element with a fluorophore enables the detection of general nonspecific hybridization by identifying cycles in which the particle is labelled with a second signal element with a fluorophore that is inconsistent with the decoder element.

In a fifth aspect the disclosure pertains to a method for generating a detection mix as disclosed herein.

In a sixth aspect the disclosure pertains method for controlling the density of the monitoring probes disclosed herein on a slide surface by attaching azide or alkyne partner to a poly-lysine before coating it. In yet a further embodiment unreactive azide or alkyne is removed, followed by a reaction step with detection mix as disclosed herein.

In a seventh aspect the disclosure encompasses a monitoring probe as described herein placed underneath a tissue in order to assess the efficacy of a tissue sample preparation process comprising in situ oligonucleotides which penetrate through the tissue and hybridize to their intended targets.

In an eighth aspect the disclosure encompasses a method to monitor temperature ranges of an in-situ environment in which monitoring probe(s) as described herein have different delta G values corresponding to different melting points of the secondary structure in order to monitor temperature fluctuations that occur during the hybridization process. In yet a further embodiment the delta G value of the monitoring probes is adjusted by the sequence length or sequence composition. In yet a further embodiment the delta G value of the monitoring probes is adjusted by using modified bases which have higher stability than standard bases. In yet a further embodiment temperature fluctuation can be read-out by specific change in the fluorescence and/or color facilitated by the direct and/or indirect interaction of the monitoring probe with the signal oligonucleotide.

The “delta G value” is defined herein as is the difference in free energy between the products and the reactants. If this value is negative, the reaction is spontaneous, if it is positive, the reaction is non-spontaneous, and if it is zero, the reaction is at equilibrium. In in-situ hybridization the delta G values corresponds to different melting points of the secondary structure and is a measure for the hybridization strength.

Thus, in a nineth aspect the disclosure encompasses a set of monitoring probes adapted to any of the methods disclosed herein.

In a tenth aspect the disclosure pertains also to a kit of parts which enable the optimization of in situ hybridization methods, comprising at least slides containing immobilized monitoring probes and/or binding sites for monitoring probes. In yet a further embodiment the kit according is adapted for monitoring a tissue sample preparation.

In an eleventh aspect the disclosure pertains to a control slide for quality control comprising a set of immobilized monitoring probes and/or binding sites for monitoring probes as described hereinunder. In yet a further embodiment the control slide can be used for assessing in-situ hybridization instrument installations.

In a twelfth embodiment uses of the monitoring probe for monitoring in situ hybridizations is used in an analytical, laboratorial, diagnostic, pharmaceutical or medical context.

### In-Situ Hybridization Multiplex Reaction

The present disclosure pertains to a reagent (monitoring probe or monitoring probe set) for methods and kits for detection of a polymorphic analyte in a sample by specific signal-encoding of said analyte by in-situ hybridization.

In one embodiment, a method for detecting a polymorphic analyte in a sample by specific signal-encoding of said polymorphic analyte is disclosed, comprises the steps of:
(A) contacting the sample with a set of polymorphic analyte-specific probes for encoding different variations of the same polymorphic analyte, each analyte-specific probe interacting with a different variation and/or sub-structure of the polymorphic analyte, wherein the polymorphic analyte is present in the sample in at least as two or more variant forms of a specific nucleic acid sequence and wherein the sequences of each polymorphic analyte are related to each other with a sequence identity of between 85% to 99%, each polymorphic analyte-specific probe comprising
(aa) a binding element(S) that specifically interacts with one of the at least one or more variants and/or sub-structure of the polymorphic analyte to be encoded, and
(bb) an identifier element (T) comprising a nucleotide sequence which is unique to the polymorphic analyte to be encoded (unique set identifier sequence), wherein each set of polymorphic analyte-specific probes differ from another set of polymorphic analyte-specific probes in the nucleotide sequence of the identifier element (T), and
(B) contacting the sample with at least a first set of decoding oligonucleotides per polymorphic analyte, wherein in each set of decoding oligonucleotides for an individual analyte each decoding oligonucleotide of the for the first set of analyte-specific probes according to step A1 comprises:
(aa) an identifier connector element (t) comprising a nucleotide sequence which is essentially complementary to at least a section of the unique set identifier sequence of the identifier element (T) of the corresponding analyte-specific probe set A1, and
(bb) a translator element (c) comprising a nucleotide sequence allowing a specific hybridization of a signal oligonucleotide;
wherein the decoding oligonucleotides of a set for an individual analyte differ from the decoding oligonucleotides of another set for a different analyte in the first connect element (t); and
(C) contacting the sample with at least a set of signal oligonucleotides, each signal oligonucleotide comprising:
(aa) a translator connector element (C) comprising a nucleotide sequence which is essentially complementary to at least a section of the nucleotide sequence of a translator element (c) comprised in a decoding oligonucleotide, and
(bb) a signal element facilitating a signal which is specific for the polymorphic analyte; and
(D) Detecting the signal caused by the signal element;
(E) Optionally selectively removing the decoding oligonucleotides and signal oligonucleotides from the sample, thereby essentially maintaining the specific binding of the analyte-specific probes to the analytes to be encoded;
(F) Performing further cycles comprising steps A) to E) for each further polymorphic analyte to generate an encoding scheme with a specific signal per set of polymorphic analytes, wherein in particular the last cycle may stop with step (D).

Depending on the design of the probe set, the detected signal may label a gene family, a single gene, a specific genetic locus, a benign or malign genetic variation, genetic specificity or biomarker in a cluster of patients, a virus-genome, a certain subset of nucleic acid variation (e.g. a group of SNPs, a mutation, a virus-variant, a gene-variant, a transcript, isoforms, splice-variants, etc.).

As such, the technology allows the high-throughput detection of nucleic acids with variable sequences of at least 60%, at least 65%, at least 70%, at least 75%, at least 80%, at least 85%, at least 90%, and up to 92%, up to 95%, up to 98%, up to 99%, up to 99.9% sequence identity. In some embodiments between 65% to 99%, between 70% to 99%, between 75% to 99%, between 80% to 99%, between 85% to 99%, between 85% to 98%, between 85% to 95%, between 90% to 95% sequence identity in a sample.

The technology is also more robust than conventional methods, since the signal is still detectable even if just a subset of probes bind the target. As such the paradigm “the label identifies the probe” is overcome and the rule “the label identifies the target” was applied instead.

In a further embodiment, this disclosure relates to in vitro methods for diagnosis of a disease selected from the group comprising cancer, neuronal diseases, cardiovascular diseases, inflammatory diseases, autoimmune diseases, diseases due to a viral or bacterial infection, skin diseases, skeletal muscle diseases, dental diseases and prenatal diseases comprising the use of the multiplex method according to the present disclosure.

In yet another embodiment, this disclosure provides in vitro methods for diagnosis of a disease in plants selected from the group comprising: diseases caused by biotic stress, preferably by infectious and/or parasitic origin, or diseases caused by abiotic stress, preferably caused by nutritional deficiencies and/or unfavourable environment, said method comprising the use of the multiplex method according to the present disclosure.

In yet another embodiment, this disclosure relates to optical multiplexing systems suitable for the method according to the present disclosure, comprising at least:
at least one reaction vessel for containing the kits or part of the kits according to the present disclosure;
a detection unit comprising a microscope, in particular a fluorescence microscope
a camera
a liquid handling device.

In yet another embodiment, this embodiment provides in vitro methods for screening, identifying and/or testing a substance and/or drug comprising:
(a) contacting a test-sample comprising a sample with a substance and/or drug
(b) detecting different analytes in a sample by sequential signal-encoding of said analytes with a method according to the present disclosure.

According to the present disclosure, unique tags (identifier) are used per target (e.g., mRNA of one single gene) or for a target group. Groups can be formed to be indicative for a certain identity, process, biological function or disease (examples: cell type, inflammation, signal processing, cancer).

Surprisingly, the methods and kits according to the present disclosure led to the reduction of complexity. Many different probes with different binding sequences share the same (one per target) unique tag. These tags have reduced the sequence complexity (to one per target) and also have predetermined constant properties (e.g., thermodynamic stability).

Advantages of the methods and kits according to the present disclosure are:
a) Full flexibility of the process to determine the identity of the tag, e.g. use more or less signals and/or rounds, varying numbers of fluorophores, number of total signals per tag→lower numbers of targets (e.g. 20) can be identified with high confidence in less rounds (e.g. 4) than a large number of targets (e.g. 100, these need 8 rounds for the same level of confidence), even if in both cases the exact same unique tags are used.
b) All unique tags are used (recycled) in many consecutive rounds of hybridization and all primary probes contribute (provide information about their identity) in every round of identification.
c) As all tags share the same predefined properties (e.g. thermodynamic stability which allows for selective denaturing).

In some advantageous embodiments, the unique tags are design as follow:
No cross-hybridization between all oligonucleotides of the process (probes, decoders, readout), so that all tag sequences are usable together (compatible)
No cross-hybridization between connector elements (bridges) of different unique tags
Stability of hybridization of the unique tags should be in a narrow range: as stable as possible (fast hybridization, i.e., short cycle times) but significantly different (in this case less stable) than the primary probe (for differential denaturation, without removing primary probes).

Therefore, the present description pertains in particular to the usage of a set of labeled and unlabeled nucleic acid sequences for specific quantitative and/or spatial detection of different analytes in parallel via specific hybridization. The technology allows the discrimination of more different analytes than different detection signals are available. The discrimination is realized via sequential signal-coding of the analytes achieved by several cycles of specific hybridization, detection of signals and selective elution of the hybridized nucleic acid sequences. In contrast to other state-of-the-art methods, the oligonucleotides providing the detectable signal are not directly interacting with sample-specific nucleic acid sequences but are mediated by so called “decoding-oligonucleotides”. This mechanism decouples the dependency between the analyte-specific oligonucleotides and the signal oligonucleotides. The use of decoding-oligonucleotides allows a much higher flexibility while dramatically decreasing the number of different signal oligonucleotides needed which in turn increases the coding capacity achieved with a certain number of detection rounds. The utilization of decoding-oligonucleotides leads to a sequential signal-coding technology that is e.g., more flexible, cheaper, simpler, faster and/or more accurate than other methods.

Furthermore, the present disclosure pertains to the use of improved decoding-oligonucleotides to increase the efficiency of the encoding scheme. The so called “multi-decoders” allows the recruiting of more than just one signal oligonucleotide and therefore can generate new signal types by utilizing the combination of two or more different signal-oligonucleotides without decreasing the brightness of the signals.

Furthermore, due to the use of a first set of analyte-specific probes according to step A1 (i.e. the transcript plexity of A1) which is at least 10 times higher in numbers than the number of probes and/or targets of the second set of analyte-specific probes according to step A2 (i.e. the transcript plexity of A2) as well as the use of at least one, preferably at least two different sets of decoding oligonucleotides for set A1 and set A2, spatially overlapping targets inside a tissue and or cell culture sample which distance is beyond the diffraction limit can be detected. Further advantages pertain to an improvement of the overall signal to noise ratio, the signal spread (i.e signals of transcripts with higher expression levels can be detected together with lowly expressed genes) and the multiplexing capability, without increasing optical crowding.

Before the disclosure is described in detail, it is to be understood that this disclosure is not limited to the particular component parts of the steps of the methods described. It is also to be understood that the terminology used herein is for purposes of describing particular embodiments only and is not intended to be limiting. It must be noted that, as used in the specification and the appended claims, the singular forms “a,” “an” and “the” include singular and/or plural referents unless the context clearly dictates otherwise. It is moreover to be understood that, in case parameter ranges are given which are delimited by numeric values, the ranges are deemed to include these limitation values.

### BRIEF DESCRIPTION OF THE DRAWINGS

FIG. 1: Embodiment where the analyte is a nucleic acid, and the probe set comprises oligonucleotides specifically binding to the analyte. The probes comprise a unique identifier sequence allowing hybridization of decoding oligonucleotides.

FIG. 2: Embodiment where the analyte is a protein, and the probe set comprises proteins (here: antibodies) specifically binding to the analyte. The probes comprise a unique identifier sequence allowing hybridization of decoding oligonucleotides.

FIG. 3: Flowchart of the method according to the disclosure.

FIG. 4: Alternative options for the application of decoding and signal oligonucleotides.

FIG. 5: Example for signal encoding of three different nucleic acid sequences by two different signal types and three detection rounds; in this example, the encoding scheme includes error detection.

FIG. 6: Number of generated code words (logarithmic scale) against number of detection cycles.

FIG. 7: Calculated total efficiency of a 5-round encoding scheme based on single step efficiencies.

FIG. 8: Comparison of relative transcript abundances between different experiments.

FIG. 9: Correlation of relative transcript abundances between different experiments.

FIG. 10: Comparison of intercellular distribution of signals.

FIG. 11: Comparison of intracellular distribution of signals.

FIG. 12: Distribution pattern of different cell cycle dependent transcripts.

FIG. 13: Detection of multiple targets using an 8 round code with 2 labels (A and B) and no label (-). The targets 1, 2, 3, 4, 5, 20, and n are represented. The rounds 1, 2, 3, and 8 of the coding scheme are represented. Herein, the blank is part of the code.

FIG. 14: Detection of multiple targets can be performed by an encoding scheme using a detectable marker. The ending scheme may comprise also the “0” as a marker. That means that at a specific position the transcript is not detected. Consequently, the encoding scheme may be represented by the following constructs using only two gene specific probes;
1) With detectable label F: detectable during imaging
2) With detectable label F and quencher Q: not detectable during imaging
3) With quencher Q: not detectable during imaging
4) Without label F: not detectable during imaging
5) Without signalling oligonucleotide: not detectable during imaging
6) With a decoder oligonucleotide that cannot recruit a signalling oligonucleotide
7) Without decoder oligonucleotide: not detectable during imaging

FIG. 15: Possible structures of a multi-decoder. The numbers depict the examples. (A) is the unique identifier sequence, (a) is the corresponding sequence of the decoding oligonucleotide or multi-decoder and (c1) to (c3) are different sequence elements, that specifically bind to different signal oligonucleotides. Examples 2 to 5 show different versions of multi decoders. The order of the different sequence elements as well as the number of signal oligonucleotide binding elements is not fixed. Example 1 shows a normal decoding oligonucleotide since there is only one signal oligonucleotide binding element (c1).

FIG. 16: Example for signal encoding of three different nucleic acid sequences by using multi-decoders and two different signal oligonucleotides creating three different signal types and three detection rounds. In this example, the encoding scheme includes error detection and correction.

FIG. 17: Number of generated codewords (logarithmic scale) against number of detection cycles. The number of codewords for merFISH does not exponentially increase with the number of detection cycles but gets less effective with each added round. In contrast, the number of codewords for intronSeqFISH, the method of the present disclosure without using multi-decoders, the method with multi-decoders increases exponentially. The slope of the curve for the method using multi-decoders is much higher than that of the prior invention, leading to more than 20 000 000 times more code words usable after 20 rounds of detection.

FIG. 18: Embodiment of a monitoring probe according to the present disclosure, wherein the monitoring probe is bound to a bead, wherein the monitoring probe comprises a target nucleic acid sequence allowing the binding/hybridization of a binding element (primary probe), wherein a decoder element (decoder) binds to the binding element and wherein a signal oligonucleotide binds to the decoder element for readout.

FIG. 19: Visual representation of mRNA and control particle.

FIG. 20: Particle attachment to microscope slights.

FIG. 21: Assay for parameter optimization,

FIG. 22: Example for the use of ZEN's DFIs.

FIG. 23: Results of MC Run of 1-plex particle

FIG. 24: Results of MC Run of 1-plex particle, 6-plex particle, 12-plex particle

FIG. 25: MC Run of 19-plex particles

FIG. 26: Embodiments of the monitoring probe according to the present disclosure. A) Monitoring probes (1) attached to a particle (3) like a magnetic bead, which is immobilized to a slide surface (2). On the right a tissue sample (4) is place on top of the particles. B) shows a specific embodiment of the particle, wherein the monitoring probe comprises a translator (c) element (1a) comprising a nucleotide sequence allowing the binding/hybridization of a signal oligonucleotide. On the right, the binding of the signal oligonucleotides (5) is shown. C) shows a specific embodiment of the particle, wherein the monitoring probe comprises an identifier element (t) (1b) comprising a nucleotide sequence allowing the binding/hybridization of a decoding element (6). On the right, the binding of the decoding elements (6) to the monitoring probe and the binding of the signal oligonucleotides to the decoding elements is shown. D) shows a specific embodiment of the particle, wherein the monitoring probe comprises a target nucleic acid sequence (1c) allowing the binding/hybridization of a binding element (7). On the right, the binding of the decoding elements (6) to the monitoring probe, the binding of the decoding element (6) and the binding of the signal oligonucleotides (5) to the decoding elements (6) is shown.

### DETAILED DESCRIPTION OF THE DISCLOSURE

Disclosed herein are novel reagents (monitoring probe and/or a set of monitoring probes) for monitoring in situ hybridization multiplex methods. One advantage of the present disclosure is that the at least one monitoring probe is part of the reaction and therefore is exposed to the same reactants and reactions steps as the in-situ hybridization multiplex analysis. Furthermore, another advantage is that the monitoring probe itself has “multiplexing-ability”, i.e., interacts directly or, preferably, indirectly with one or more signal oligonucleotides via decoding elements. This allows a combinatorial variation which allows to generate a readout which is far more complex and carries much more information, by the use of still only a few elements, than conventional monitoring methods. This “multiplexing-ability” allows for example to adjust the signal intensity to the respective in situ hybridization multiplex analysis; allows to monitor intermediate steps of the reaction in different colours and/or allows fluctuations in the reaction process, e.g., temperature fluctuations. The at least one monitoring probe can be present in solution and/or immobilized on a surface, for example a reaction matrix, a column, a slide, and the like.

In particular, the present disclosure pertains to methods of ground truth generation for detection optimization and transcript classification.

DNA, RNA or other molecules within a biological tissue sample can be detected within their spatially oriented environment, using fluorescence imaging techniques, such as smFISH. Recent development in combinatorial smFISH chemistry, e.g. the spatial biology methods of the present disclosure enable parallel detection of large number of such molecules within cells or tissues. Such detection system has been demonstrated to identify biologically relevant transcripts (RNAs) with high sensitivity and low error rate. However, the actual quantities of the target molecules remain unknown, as no prior tools exist to do absolute quantification of molecules within cells or tissue samples while preserving the spatial orientation of such samples. Due to the lack of actual quantities of targets under measurement, the sensitivity (true positive #/(true positive #+false negative #)) of such measurement has only been estimated using detected events using traditional methods. For example, the number of a RNA target detected by smFISH has been used as a reference for 100% detectable targets within cells or tissue samples. However, multiple MC data sets have shown higher number of detectable RNA targets than what smFISH quantifies. In addition, the smFISH quantification methods rely on subjective settings of intensity threshold, making the ground truth reference a moving target. In this document, multiple methods are described to generate the actual number of targets or ground truth information, which can be used for the optimization of detection and classification.

In particular, the detection methods according to the present disclosure uses oligo hybridization and de-hybridization to RNA targets to create on states (fluorophores attached) and off states in multiple cycles. Within each cycle, fluorescence images of the cells and tissues samples are collected. Image analysis is performed to identify the on and off states of measured locations within the samples. The specific sequence of the on and off states associated with a target can be considered as a barcode for unique identification of the target. The deviation of the on and off states for any location under measurement is the results of many factors, such as integrity of RNA targets within the sample, oligos' hybridization reaction completeness, none-specific binding of fluorophores, image quality and uniformity from the optics used, camera pixels' temporal noise, etc. Due to the complex mechanism to generate the on and off states, it is critical to generate the ground truth with reduced complexity.

In some advantageous embodiments, the present disclosure pertains to oligo attached beads (monitoring probes) and uses thereof to create ground truth. The oligos attached to the beads can be
a) same oligos as the decoder tails (translator element (c)) that binds to signal oligonucleotides;
b) same oligos as ‘tails’ on gene specific probes (identifier element (t)) for binding with decoder elements;
c) combination of the designed target sequences on a RNA.

The different configurations allow the oligo beads to generate on and off states that associated with different complexity of the hybridization chemistry. The quantity of such beads can be controlled by the concentration of the bead solution. The beads can be deposited onto a glass slide. The beads can be quantified by other measurement techniques, e.g. bright field or dark field microscope imaging, to set the ground truth. Tissue samples can be placed on top of these beads. Some or all of these species with or without tissue samples on top can be measured and quantified against the ground truth information obtained. The information can be used to identify each step of the assay, e.g. by measuring counts of gene specific oligo combo beads without tissue on top and then with tissue on top, one can quantify oligo penetration and hybridization reaction completeness.

Some further embodiments of the present disclosure pertain to the use of computer simulated signal values to create ground truth. Measured signal values from actual known targets can be used to simulate the ground truth. The simulated signal values can be infused within a data set with certain spatial distribution or at specific locations of interest. The on and off state can be set to match barcodes in use or not in use. The signal values can be adjusted to low and high levels for optimization. The signals can also be generated in low and high spatial density levels with known separations. The flexible parameter setting of the ground truth information provides image analysis development absolute reference for event classification, model training and optimization.

Some further embodiments of the present disclosure pertain to the use of known quantity targets within the biological sample, e.g. DNA targets. DNA FISH targets of known species can be used as the ground truth, as the number of copies is known. Detecting such targets within the tissue sample provides absolute calibration of the detection system.

In some advantageous embodiments, the monitoring probe comprises at least one identifier element (“tail”) which unique oligonucleotide-sequence allows the specific recognition by at least one decoding element via selective hybridization. The decoding element then allows the detection of a signal oligonucleotide, either directly, or by a chain of two, three, four or more decoding elements. The signal oligonucleotide then provides an optical signal, which could be enzymatic, a dye or, preferably a fluorescent signal.

The monitoring probe can be present either in solution or is attached to a surface, either directly or via a particle, such as a magnetic bead, a biotin-tag, a strep-tag, or the like. In one embodiment the oligonucleotide comprises a spacer sequence between the identifier element (“tail”) and the part of the oligonucleotide which is attached to the surface and/or bead. In one embodiment the monitoring probe is immobilized on the same slide on which the in-situ reaction takes place.

As such the disclosure describes a monitoring probe (“control particle”) that can be used for the following purposes:
1. Quality control (QC) and release of kit components and final kit
2. Evaluation of workflows and validation of software and hardware updates.
3. Performance of instrument qualification during installation or instrument repair.
4. Internal standard for customer runs
5. Training of customers new to the in-situ hybridization workflow.
6. Validating that a customer can perform the protocol prior to running clinical samples in a diagnostic test.
7. Evaluation of the efficacy of tissue preparation to enable diffusion of detection sequences into (and through) the tissue.

In one embodiment, the final monitoring reagent consists of a set of monitoring probes, each monitoring probe having a different attached identifier element. In the examples used in this disclosure, the identifier elements attached to the particles are referred to as “tail” sequences. Each of the tail sequences is complementary to one of the decoder sequences. The combination of different decoder pools and cycles of detection allows for multiplexing of the different unique elements into a control particle pool. At the end of the run, the identity of each particle can be determined by the sequence and colour of fluorescence at each cycle. Alternative to using tail sequences, the attached sequence could be a unique sequence analogous to a targeted RNA. This would enable monitoring the entire workflow, including the overnight steps needed to hybridize the probe sets to the tissue. Alternatively, the sequence attached to the particle could be complementary to the readout probe. This would enable the particle to serve as a control to the final steps involved in binding and washing the readout probe.

The use of magnetic particles as shown in the examples hereinunder facilitate manufacturing and washing of the control beads using a magnetic field for separation. Alternatively, nonmagnetic particles can be used. When using nonmagnetic particles, the binding and washing steps can be accomplished using standard batch or chromatographical processes. Alternatively, the sequences could be attached to the slide by spotting directly.

To serve as a relevant control, the number of attached oligonucleotides needs to be comparable to the number of tails that would be present on a targeted mRNA analyte. Having too many oligonucleotides per particle would result in fluorescent signals that generates images of inappropriate size and/or intensity. Although the number of tails attached per particle can be achieved by titrating the concentration of tails during attachment, a more controlled method would be using a decoy. When using streptavidin-coated particles, decoys can consist of biotinylated oligonucleotide, free biotin, or any moiety able to bind to the biotin binding site. Using a decoy to control the final number of tails attached to the particle results in a more robust manufacturing process and allows saturation of the particle binding sites independent of the number of particles in the preparation.

Each element in the set of control particles will have a unique sequence attached to the surface. Each element can be manufactured, and the quality control can take place in bulk. Once validated, the individual elements can be pooled to generate the final control particle pool. Production of the particles in solution facilitates QC of the individual elements as well as the final pool of control particles.

The attachment of the oligonucleotide to the particle can be accomplished by a variety of chemistries, as is standard in the art. Attachment using biotinylated oligonucleotide and streptavidin-coated particles was the method selected in this invention. To ensure that the particle remains attached to the slide during the steps required to perform molecular cartography, the beads can be attached to the slide using a variety of chemistries. For example, when streptavidin-coated particles are used, the biotin moieties can be attached to the slide surface that will bind to biotin-binding sites on the particle's surface. Alternatively, reactive moieties can be added to the surface of the slide either directly or by attaching to the particle additional oligonucleotides that contain reactive groups such as alkyne or azides that enable click chemistry for covalent attachment.

One approach to manufacturing the control slides is to activate the particle with reactive group, such as an azide or alkyne. A second reactive group, such as the alkyne or azide, can be attached to a coating polymer (Poly lysine) in solution. Activating a coating polymer in solution allows for more controlled manufacturing and the ability to QC and characterize the bulk material. One approach would be to coat the slide surface with the bulk activated polymer followed by attachment of the particle to the coated slide surface. Alternatively, the activated bulk polymer can react with the activated particle followed by coating the particle-polymer complex to the slide surface.

The tails and decoders can be designed to monitor environmental conditions that may influence the performance of the platform. By designing a series of sequences covering different delta G values, the control particles will be able to detect instrument or environmental temperatures that fall outside of the desired range of the assay. By designing decoder sequences with different delta G values at either end, the environmental temperatures at different steps of the hybridization workflow can be monitored by the workflow. Altered delta G values can be accomplished by selecting the base composition of the sequence, altering the sequence length or the use of modified bases such as LNA, PNA, Methyl phosphonates, C-5 propynyl derivatives, or incorporation of minor-group binding dyes.

Quantification of hybridization efficiencies can be done in many ways. The attached tail can be fluorescently labeled with an orthogonal fluorophore (fluorophore 2) to quantify the amount of tail attached to each individual particle. Comparing the amount of fluorescent readout probe (fluorophore 1) to the amount of attached tail (fluorophore 2), enables quantification of hybridization efficiency. The orthogonal fluorophore can be used to label the particle directly, the attached tail or the decoder. Depending on which moiety is labeled, various stages of the hybridization process can be quantified.

Slides coated with particles can be used directly without requiring any biological sample. Such control slides can be used for:
1. QC and release of kit reagents
2. Evaluation of workflows and validation of software and hardware updates.
3. Performance of instrument qualification during installation or instrument repair.
4. Training of customers new to the in-situ hybridization workflow.
5. Validating that a customer can perform the protocol prior to running clinical samples in a diagnostic test.

Alternatively, customer samples can be applied directly to slides containing attached control beads. In these cases, the control bead will:
1. Serve as an internal control to monitor the entire workflow used by the customer in running their sample.
2. Monitor the efficiency of the tissue preparation process in permeabilizing the tissue sufficiently to allow diffusion of the assay oligonucleotides into and through the tissues.

The present disclosure also describes the usage of at least one set of nucleic acid sequences for specific quantitative and/or spatial detection of different analytes in parallel via specific hybridization. The technology allows the discrimination of more different analytes than different detection signals are available. The discrimination may be realized via sequential signal-coding of the analytes achieved by several cycles of specific hybridization, detection of signals and selective elution of the hybridized nucleic acid sequences.

In contrast to other state-of-the-art methods, the oligonucleotides providing the detectable signal are not directly interacting with sample-specific nucleic acid sequences but are mediated by so called “decoding-oligonucleotides”. This mechanism decouples the dependency between the analyte-specific oligonucleotides and the signal oligonucleotides. The use of decoding-oligonucleotides allows a much higher flexibility while dramatically decreasing the number of different signal oligonucleotides needed which in turn increases the coding capacity achieved with a certain number of detection rounds.

Furthermore, in contrast to other state-of-the-art methods using already “decoding-oligonucleotides”, at least one, preferably at least two different sets of “decoding-oligonucleotides” are used directed to each of the at least one, preferably at least two different analytic sets, in order to allow the detection of different subgroups of targets within one analytical run and that any spatial resolving limitation no longer applies.

In one embodiment, the analytical rounds can be arranged consecutively, meaning the detection round(s) of a first analytical set is finished before the detection round(s) of a second analytical set start.

In a second embodiment of the method of the present invention, the analytical rounds can be arranged interleaved, meaning the detection rounds of a first analytical set and a second analytical set alternate in a certain pattern, e.g.:
Detection round 1=1st round of detection of first analytical set
Detection round 2=1st round of detection of second analytical set
Detection round 3=2nd round of detection of first analytical set
Detection round 4=2nd round of detection of second analytical set
Etc.

The utilization of decoding-oligonucleotides leads to a sequential signal-coding technology that is more flexible, cheaper, simpler, faster and/or more accurate than other methods and allows a resolution beyond the diffraction limit of optical microscopes.

### DEFINITIONS

According to the present disclosure an “slide” is any surface which can be used for in-situ hybridization. In some embodiments the slide is a microscope slide, in some embodiments made out of glass.

According to the present disclosure an “analytical set” is a set of probes specific for a subgroup of targets. Preferably the targets within a first analytical set directed to a subgroup of targets which do not usually show a high spatial overlap, whereas a second analytical set directed to a subgroup of targets which may spatially overlap with the first subgroup and/or may add additional information which allows a further differentiation of the signals found with the first analytical set. For example, a first analytical set may be directed to known activating mutations in promotor-structures with a high prevalence for cancer. The second analytical set may be directed to genes associated with cancer development when over-expressed. Thus, a colocalization of signals detected with the first and the second analytical set may indicate activated promotors in genes associated with cancer. Of course also other combinations can be envisaged, for example tissue-type-specific probes with cancer markers, proto-oncogenic targets in different combinations, etc.,

According to the present disclosure an “analyte” is the subject to be specifically detected as being present or absent in a sample and, in case of its presence, to encode it. It can be any kind of entity, including a protein, polypeptide, protein, or a nucleic acid molecule (e.g. RNA, PNA or DNA) of interest. The analyte provides at least one site for specific binding with analyte-specific probes. Sometimes herein the term “analyte” is replaced by “target”. An “analyte” according to the disclosure incudes a complex of subjects, e.g., at least one, preferably at least two individual nucleic acid, protein or peptides molecules. In an embodiment of the disclosure an “analyte” excludes a chromosome. In another embodiment of the disclosure an “analyte” excludes DNA.

In some embodiments, an analyte may be a “coding sequence”, “encoding sequence”, “structural nucleotide sequence” or “structural nucleic acid molecule” which refers to a nucleotide sequence that is translated into a polypeptide, usually via mRNA, when placed under the control of appropriate regulatory sequences. The boundaries of the coding sequence are determined by a translation start codon at the 5′-terminus and a translation stop codon at the 3′-terminus. A coding sequence can include, but is not limited to, genomic DNA, cDNA, EST and recombinant nucleotide sequences.

A “sample” as referred to herein is a composition in liquid or solid form suspected of comprising the analytes to be encoded. In particular, the sample is a biological sample, preferably comprising biological tissue, further preferably comprising biological cells and/or extracts and/or part of cells. For example, the cell is a prokaryotic cells or a eukaryotic cell, in particular a mammalian cell, in particular a human cell. In some embodiments, the biological tissue, biological cells, extracts and/or part of cells are fixed. In particular, the analytes are fixed in a permeabilized sample, such as a cell-containing sample.

As used in the present disclosure, “cell”, “cell line”, and “cell culture” can be used interchangeably and all such designations include progeny. Thus, the words “transformants” or “transformed cells” include the primary subject cell and cultures derived therefrom without regard for the number of transfers. It is also understood that all progeny may not be precisely identical in DNA content, due to deliberate or inadvertent mutations. Mutant progeny that have the same functionality as screened for in the originally transformed cell are included.

An “encoding scheme” may describe a set of code words that are associated with the analytes to be detected. Each code word refers to one of the analytes and can be distinguished from all other code words. A code word hereby is a sequence of signs provided by the detection cycles of the method. A sign within a code word is a detectable signal or the absence of a signal. A code word does not need to comprise of all different signals used in the method. The number of signs in a code word is defined by the number of detection cycles.

An “oligonucleotide” as used herein, refers to s short nucleic acid molecule, such as DNA, PNA, LNA or RNA. The length of the oligonucleotides is within the range 4-200 nucleotides (nt), preferably 6-80 nt, more preferably 8-60 nt, more preferably 10-50 nt, more preferably 12 to 35 depending on the number of consecutive sequence elements. The nucleic acid molecule can be fully or partially single-stranded. The oligonucleotides may be linear or may comprise hairpin or loop structures. The oligonucleotides may comprise modifications such as biotin, labeling moieties, blocking moieties, or other modifications.

The “analyte-specific probe” consists of at least one, preferably at least two elements, namely the so-called binding element(S) which specifically interacts with one of the analytes, and a so-called identifier element (T) comprising the ‘unique identifier sequence’. The binding element(S) may be a nucleic acid such as a hybridization sequence or an aptamer, or a peptidic structure such as an antibody.

The “monitoring probe” comprises at least one element, wherein this element may be
a translator (c) element according to the present disclosure, wherein the translator (c) comprises a nucleotide sequence allowing the binding/hybridization of a signal oligonucleotide;
an identifier element (t) comprising a nucleotide sequence allowing the binding/hybridization of a decoding element;
a target sequence allowing the binding/hybridization of a binding element according to the present disclosure.

In particular, the “monitoring probe” consists of at least one element, namely the so-called identifier element (T) comprising the ‘unique identifier sequence’. However, in further embodiments the monitoring probe is attached to a surface, selected from a particle (e.g., a magnetic or nonmagnetic bead), a functional group (e.g., streptavidin, biotin, a hybridization sequence or an aptamer, or a peptidic structure such as an antibody) and a surface (e.g., a slide), as well as any combination thereof. Thus, in one embodiment the monitoring probe may also comprise a so-called binding element(S) which specifically interacts with a target sequence.

In particular, in some embodiments the binding element(S) comprises moieties which are affinity moieties from affinity substances or affinity substances in their entirety selected from the group consisting of antibodies, antibody fragments, receptor ligands, enzyme substrates, lectins, cytokines, lymphokines, interleukins, angiogenic or virulence factors, allergens, peptidic allergens, recombinant allergens, allergen-idiotypical antibodies, autoimmune-provoking structures, tissue-rejection-inducing structures, immunoglobulin constant regions and their derivatives, mutants or combinations thereof. In further advantageous embodiments, the antibody fragment is a Fab, an scFv; a single domain, or a fragment thereof, a bis scFv, Fab2, Fab3, minibody, maxibody, diabody, triabody, tetrabody or tandab, in particular a single-chain variable fragment (scFv).

The “unique identifier sequence” as comprised by the analyte-specific probe and the monitoring probe is unique in its sequence compared to other unique identifiers. “Unique” in this context means that it specifically identifies only one analyte, such as Cyclin A, Cyclin D, Cyclin E etc., or, alternatively, it specifically identifies only a group of analytes, independently whether the group of analytes comprises a gene family or not. Therefore, the analyte or a group of analytes to be encoded by this unique identifier can be distinguished from all other analytes or groups of analytes that are to be encoded based on the unique identifier sequence of the identifier element (T). Or, in other words, there is only one ‘unique identifier sequence’ for a particular target sequence, analyte or a group of analytes, but not more than one, i.e., not even two. Due to the uniqueness of the unique identifier sequence the identifier element (T) hybridizes to exactly one type of decoding oligonucleotides. The length of the unique identifier sequence is within the range 8-60 nt, preferably 12-40 nt, more preferably 14-20 nt, depending on the number of analytes encoded in parallel and the stability of interaction needed. A unique identifier may be a sequence element of the analyte-specific probe, attached directly or by a linker, a covalent bond or high affinity binding modes, e.g., antibody-antigen interaction, streptavidin-biotin interaction etc. It is understood that the term “analyte specific probe” includes a plurality of probes which may differ in their binding elements(S) in a way that each probe binds to the same analyte but possibly to different parts thereof, for instance to different (e.g. neighboring) or overlapping sections of the nucleotide sequence comprised by the nucleic acid molecule to be encoded. However, each of the plurality of the probes comprises the same identifier element (T).

It is understood that the term “monitoring probe” includes a plurality of probes which may differ in their identifier element (T) in a way that each probe binds to the same decoder element and/or signal oligonucleotide, but possibly to different parts thereof, for instance to different (e.g. neighbouring) or overlapping sections of the nucleotide sequence comprised by the nucleic acid molecule to be encoded. However, in at least one embodiment, each of the plurality of the probes comprises the same binding element(S).

A “bipartite labeling probe” comprise a binding sequence capable of hybridizing the analyte and a binding probe sequence capable of binding a detectable signal molecule like a fluorophore or a nucleic acid sequence comprising a fluorophore.

A “decoding oligonucleotide” or an “adapter” or a/adapter segment” consists of at least one, preferably at least two sequence elements. One sequence element that can specifically bind to a unique identifier sequence, referred to as an “identifier connector element” (t) or “first connector element” (t), and a second sequence element specifically binding to a signal oligonucleotide, referred to as “translator element” (c). The length of the sequence elements is within the range 8-60 nt, preferably 12-40 nt, more preferably 14-20 nt, depending on the number of analytes to be encoded in parallel, the stability of interaction needed, and the number of different signal oligonucleotides used. The length of the two sequence elements may or may not be the same.

In some advantageous embodiments, the decoding oligonucleotide in the kits and/or methods of the present disclosure may be a “multi-decoder”. A “multi-decoder” is a decoding oligonucleotide that consists of at least three sequence elements. One sequence element (the identifier connector element (t)) can specifically bind to a unique identifier sequence (identifier element (T)) and at least one, preferably at least two other sequence elements (translator elements (c)) specifically bind different signal oligonucleotides (each of these sequence elements specifically binds a signal oligonucleotide that differs to all other signal oligonucleotides recruited by other elements of the multi-decoder). The length of the sequence elements is within the range 8-60 nt, preferably 12-40 nt, more preferably 14-20 nt, depending on the number of analytes detected in parallel, the stability needed and the number of different signal oligonucleotides used. The length of the sequence elements may or may not be the same.

Therefore, in some advantageous embodiments, the decoding oligonucleotide is a multi-decoder comprising
an identifier connector element (t) comprising a nucleotide sequence which is essentially complementary to at least a section of the unique identifier sequence of the identifier element (T) of the corresponding analyte-specific probe set, and
at least one, preferably at least two translator elements (c) comprising each a nucleotide sequence allowing a specific hybridization of a different signal oligonucleotide.

Therefore, the first translator element binds a different signal oligonucleotide as the second translator element. In particular, the signal oligonucleotides differ in the signal element comprised in the signal oligonucleotide, e.g. in the kind of the fluorophore.

A “signal oligonucleotide” or a “reporter” as used herein comprises at least one, preferably at least two elements, a so-called “translator connector element” (C) or “second connector element” (C) having a nucleotide sequence specifically hybridizable to at least a section of the nucleotide sequence of the translator element (c) of the decoding oligonucleotide, and a “signal element” which provides a detectable signal. This element can either actively generate a detectable signal or provide such a signal via manipulation, e.g. fluorescent excitation. Typical signal elements are, for example, enzymes that catalyze a detectable reaction, fluorophores, radioactive elements or dyes.

A “set” refers to a plurality of moieties or subjects, e.g., analyte-specific probes, monitoring probes, or decoding oligonucleotides, whether the individual members of said plurality are identical or different from each other. In an analyte specific probe set, the analyte specific probes are identical in the identifier element (T) but may comprise a different binding element(S) for specifically interacting with the same analyte but for specifically interacting with different sub-structures of the same analyte to be encoded. In a monitoring probe set, the monitoring probes are identical in the binding element(S) but may comprise a different identifier element (T) for specifically interacting with the surface on which they are immobilized, but for specifically interacting with different decoding elements. In some embodiments, the set may further comprise elements, such as a decoy, which may have a similar structure as the monitoring probe but may not comprise the same identifier element (T) in order to improve the signal-to-noise ratio.

“Selective denaturation” may be the process of eliminating bound decoding oligonucleotides and signal oligonucleotides with highest efficiency while at the same time the target specific probes have to stay hybridized with the highest efficiency. The total efficiency of these two combined events may to be at least 0.22 for two detection cycles, 0.37 for three detection cycles, 0.47 for four detection cycles, 0.55 for five detection cycles, 0.61 for six detection cycles, 0.65 for seven detection cycles, 0.69 for eight detection cycles, 0.72 for nine detection cycles and 0.74 for 10 detection cycles, 0.76 for 11 detection cycles and 0.78 for 12 detection cycles.

In an embodiment of the disclosure a single set refers to a plurality of oligonucleotides.

An “analyte specific probe set” refers to a plurality of moieties or sub-jects, e.g., analyte-specific probes that are different from each other and bind to independent regions of the analyte. A single analyte specific probe set is further characterized by the same unique identifier.

A “decoding oligonucleotide set” refers to a plurality of decoding oligonucleotides specific for a certain unique identifier needed to realize the encoding independent of the length of the code word. Each and all of the decoding oligonucleotides included in a “decoding oligonucleotide set” bind to the same unique identifier element (T) of the analyte-specific probe.

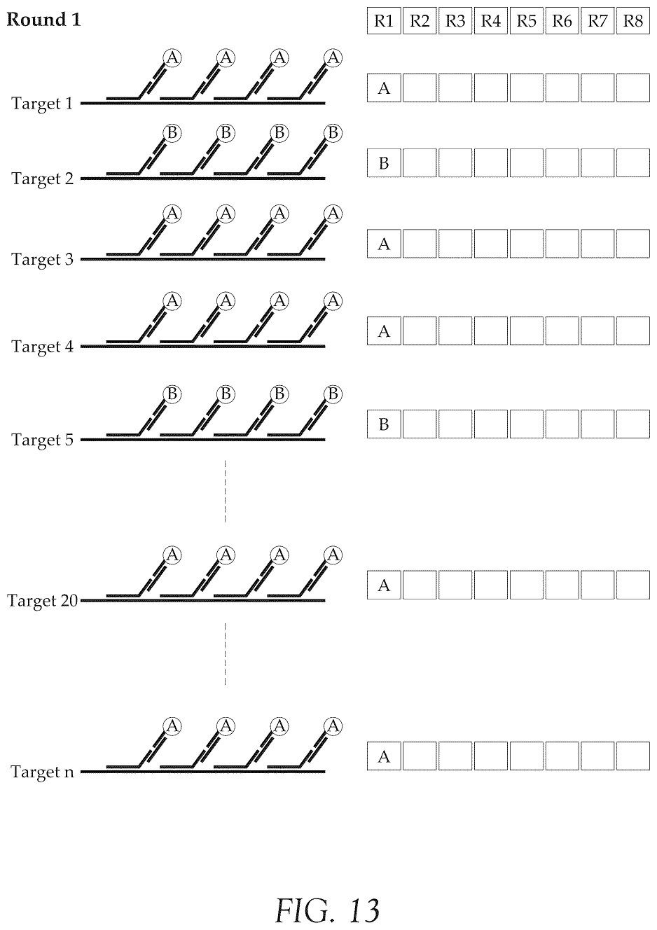

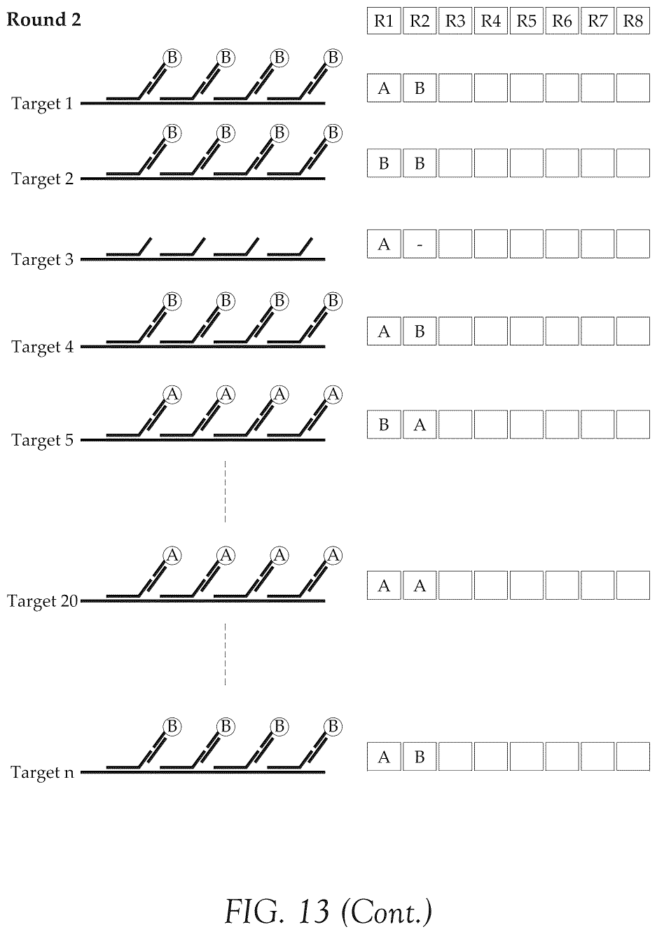

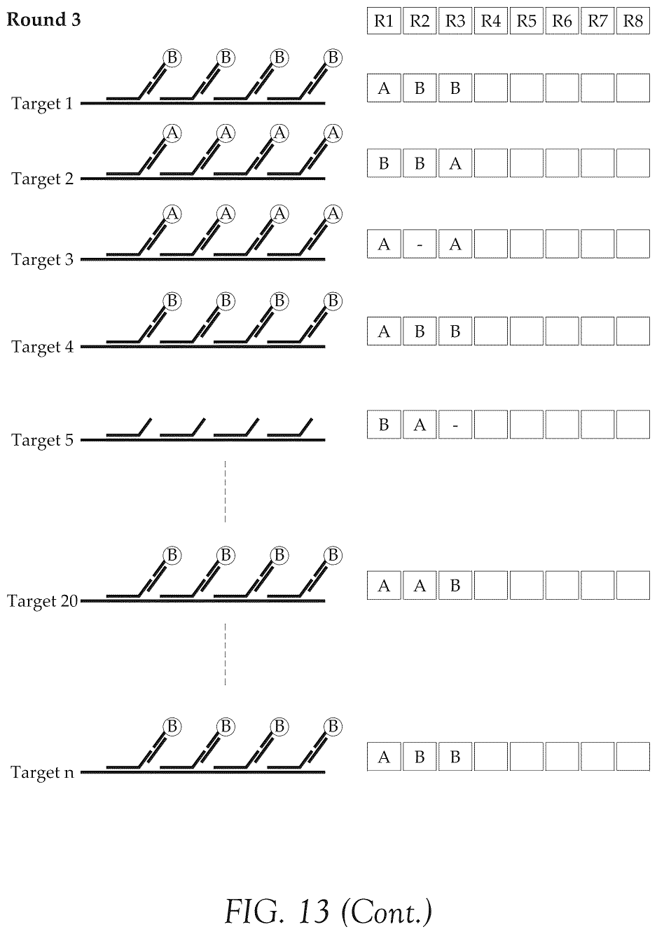

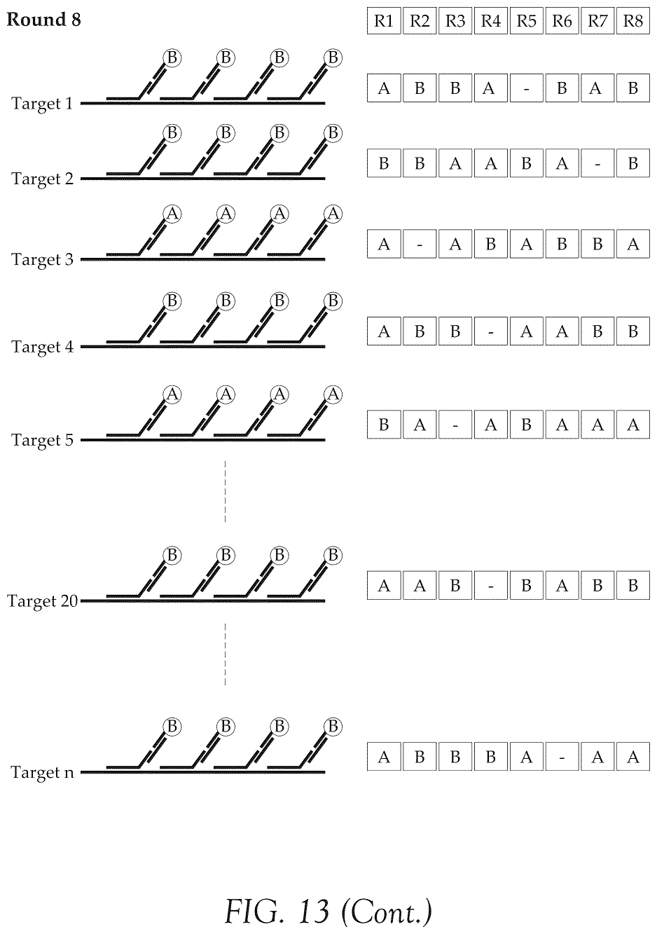

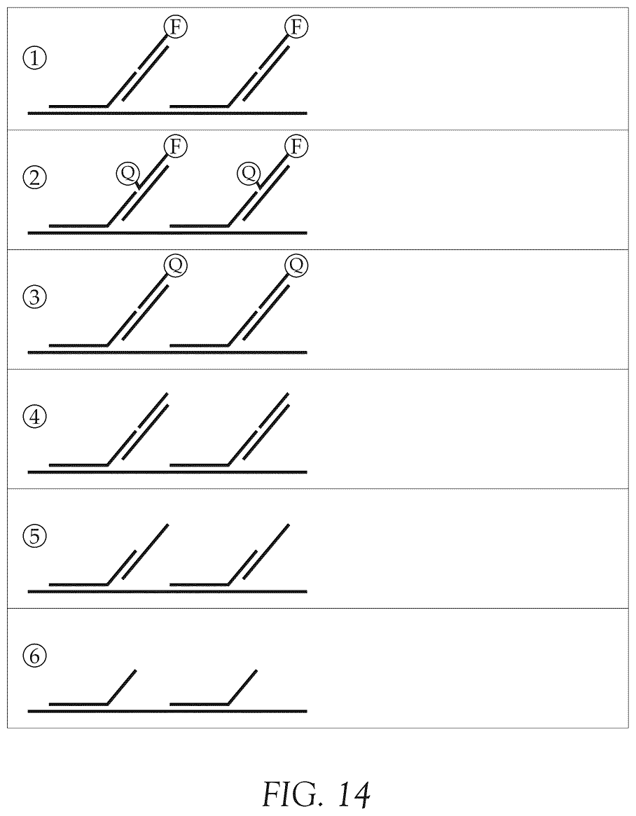

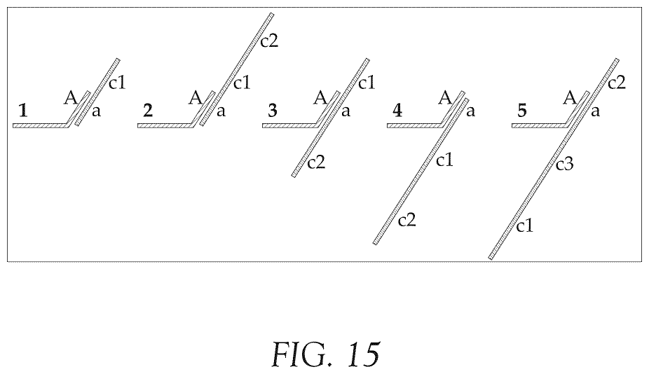

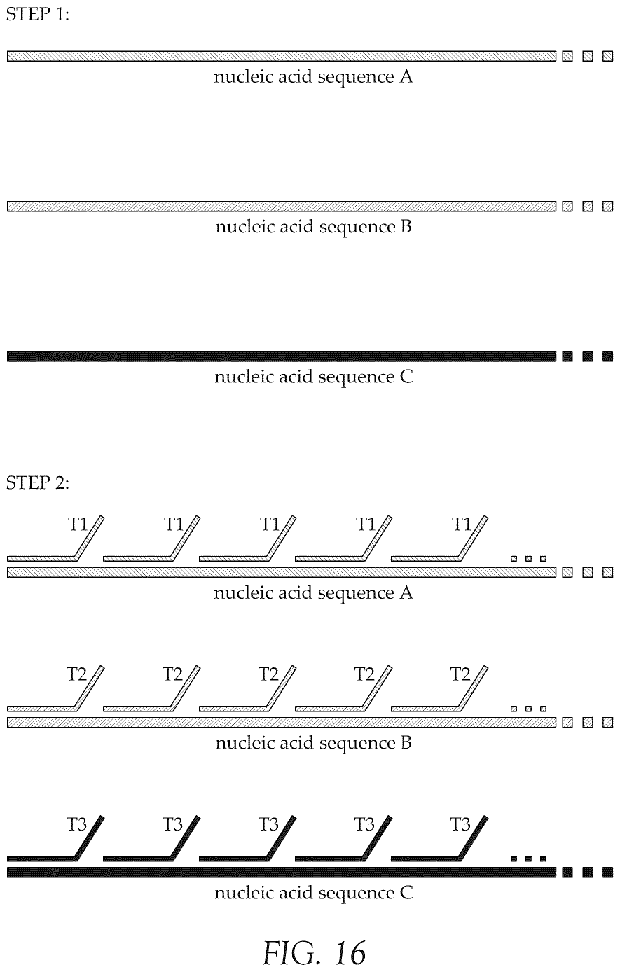

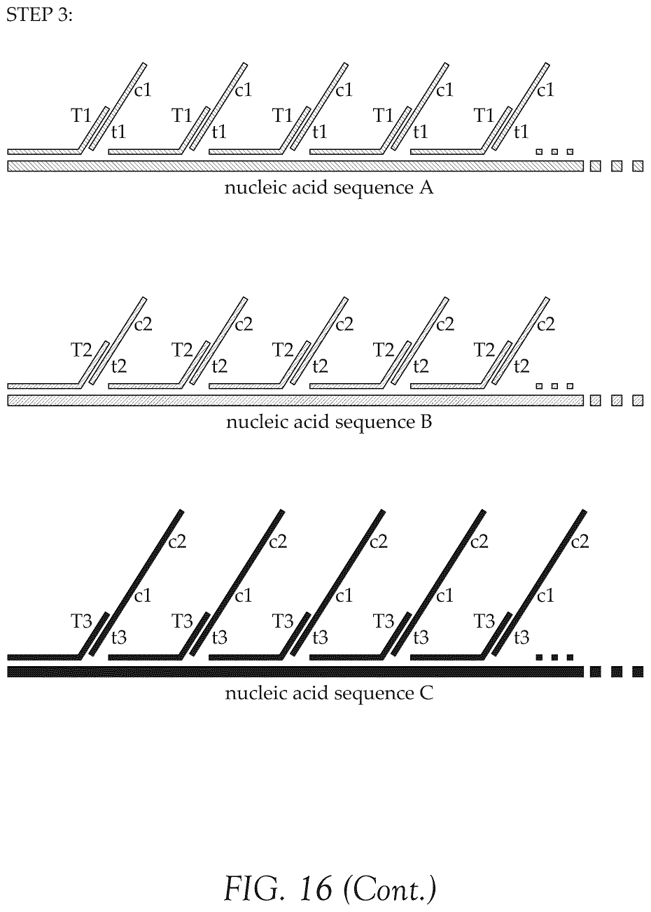

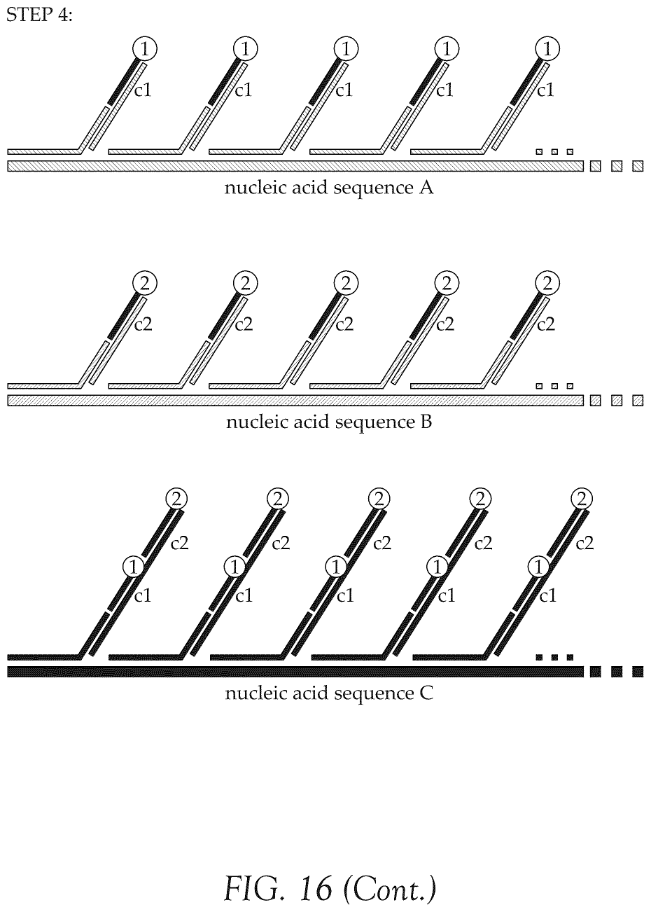

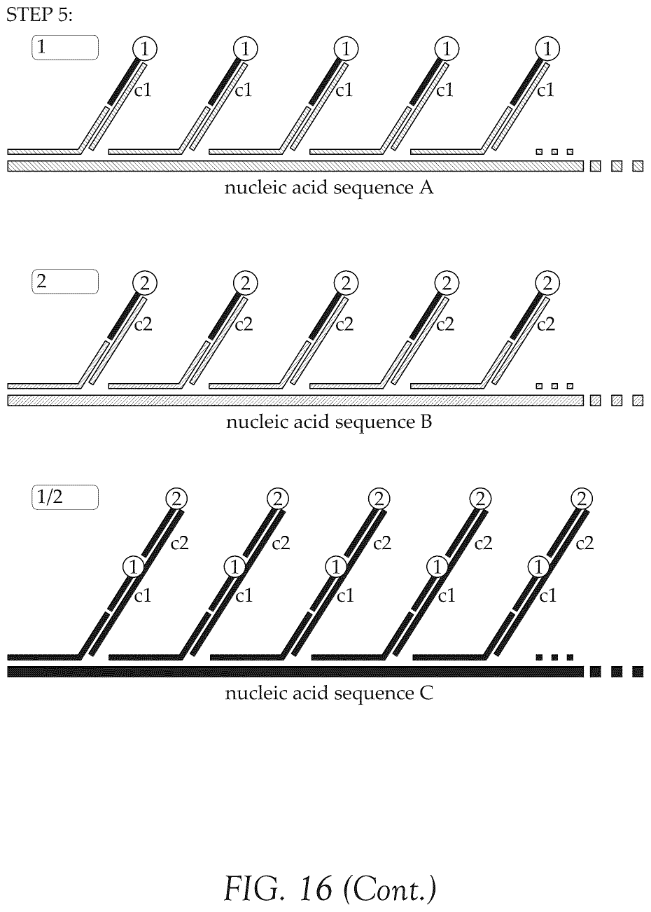

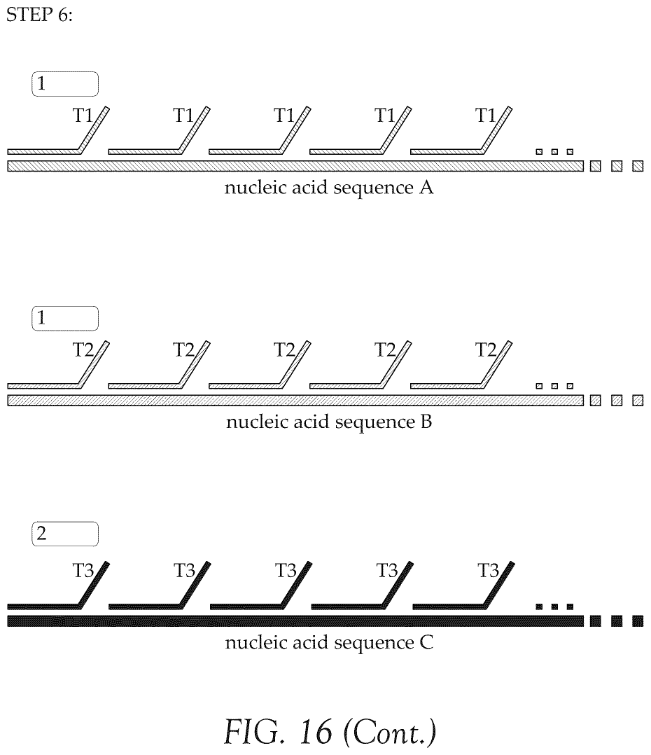

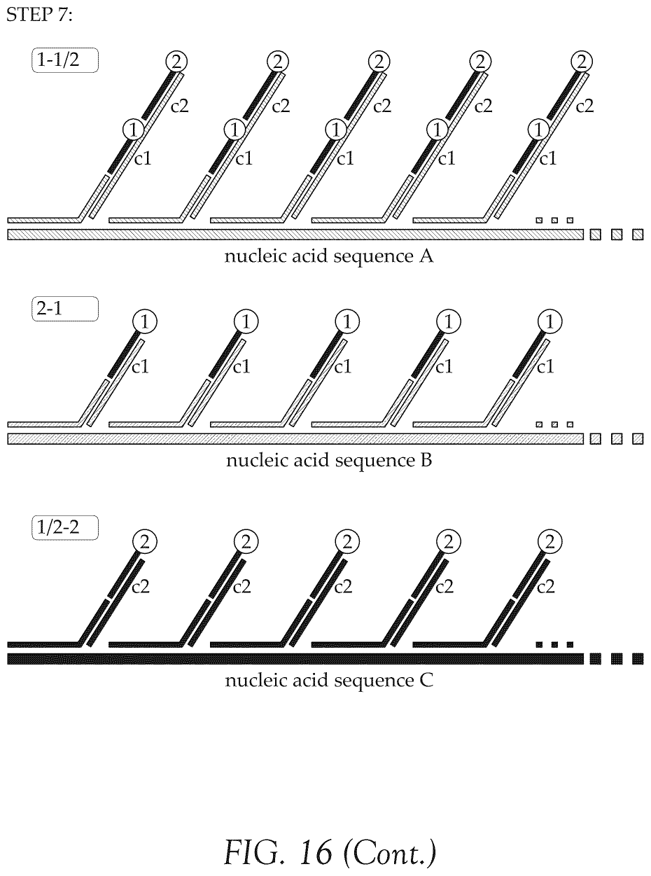

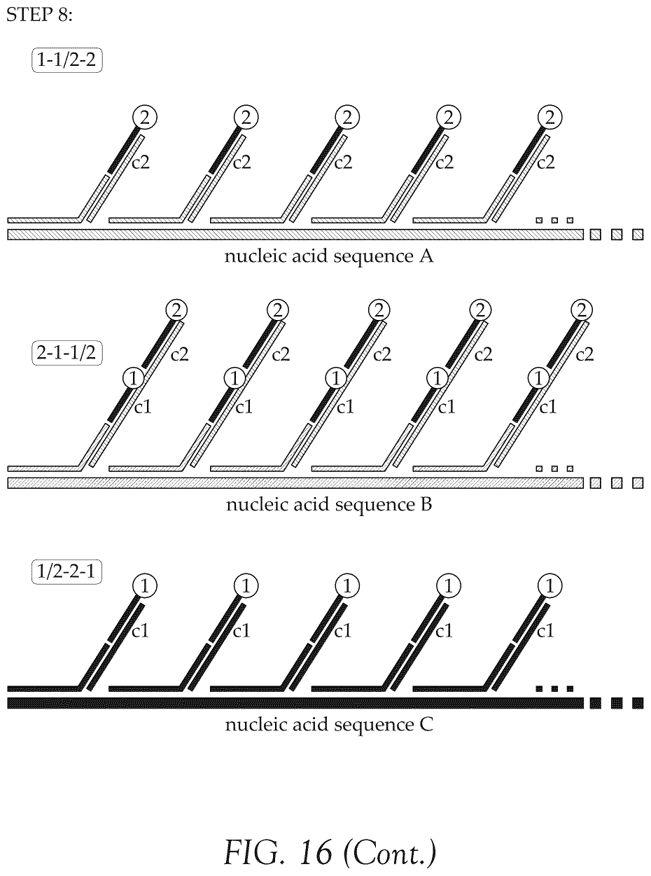

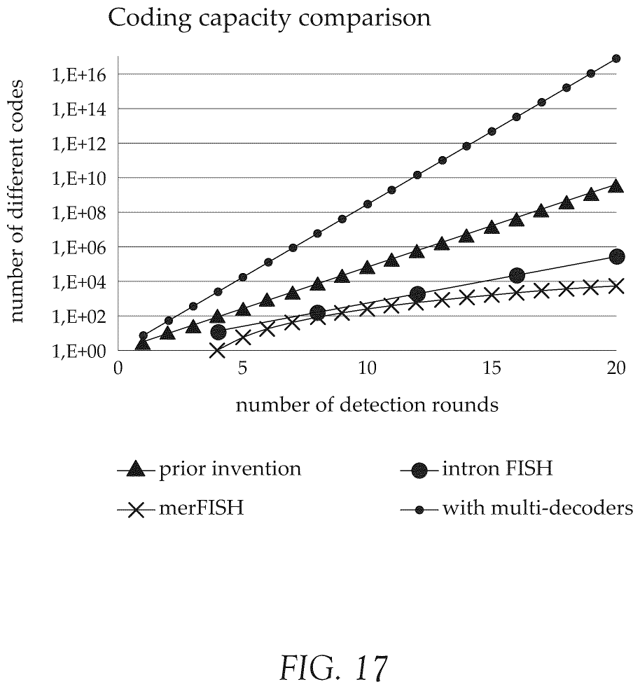

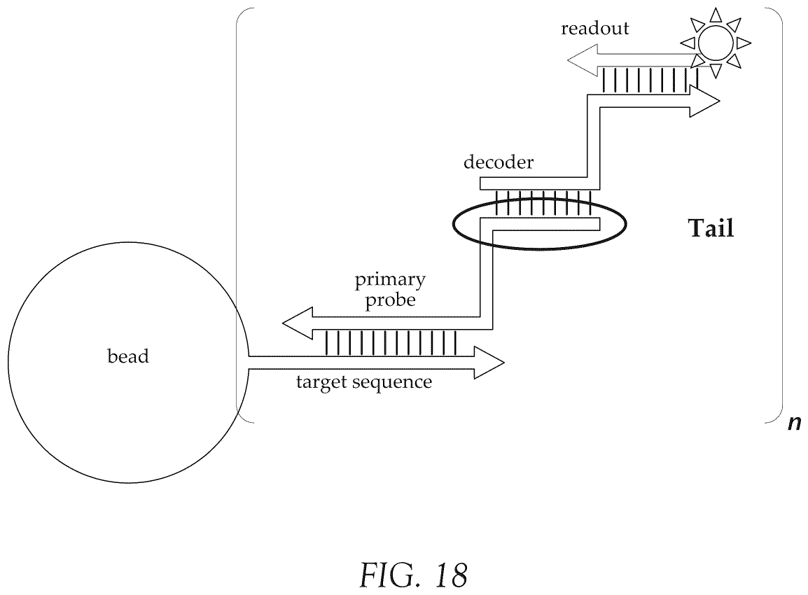

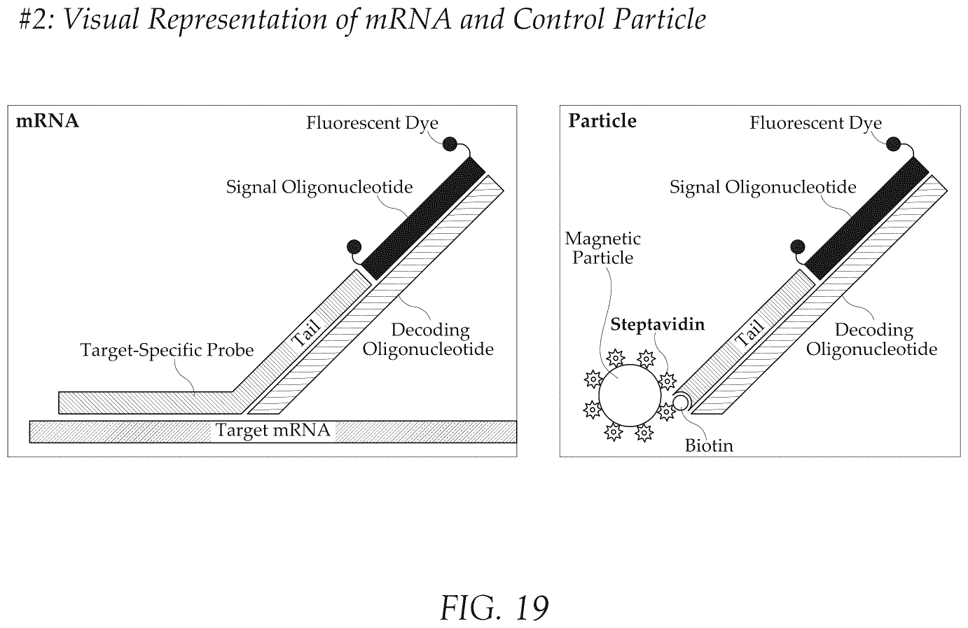

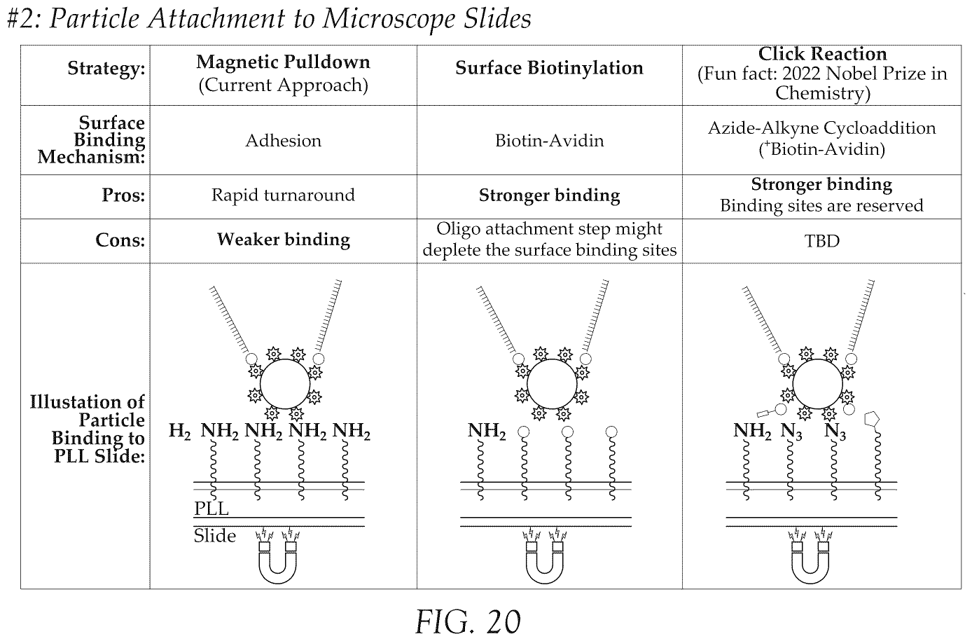

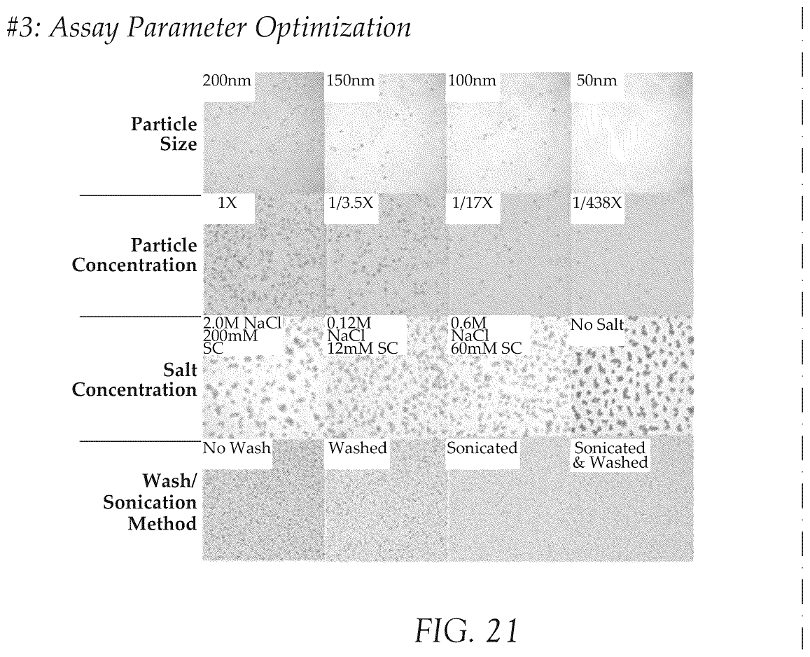

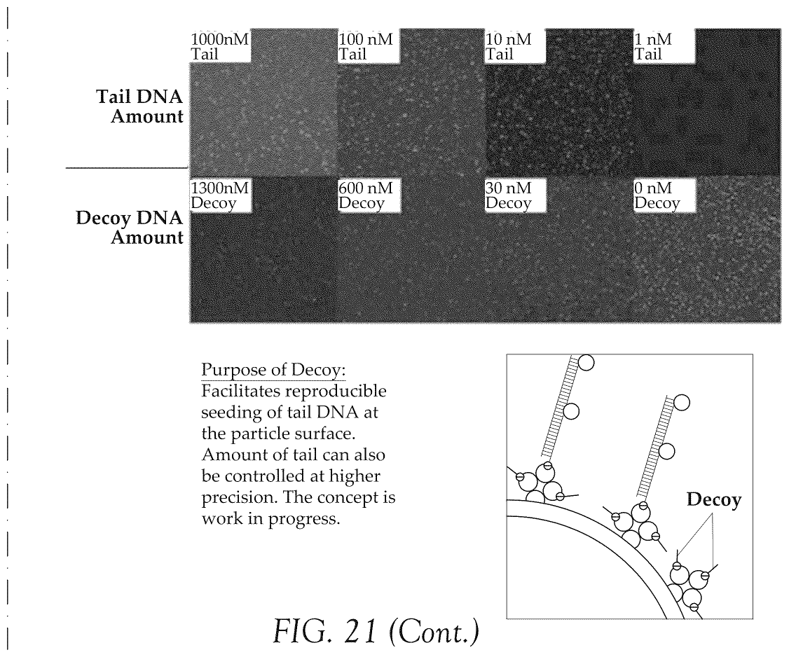

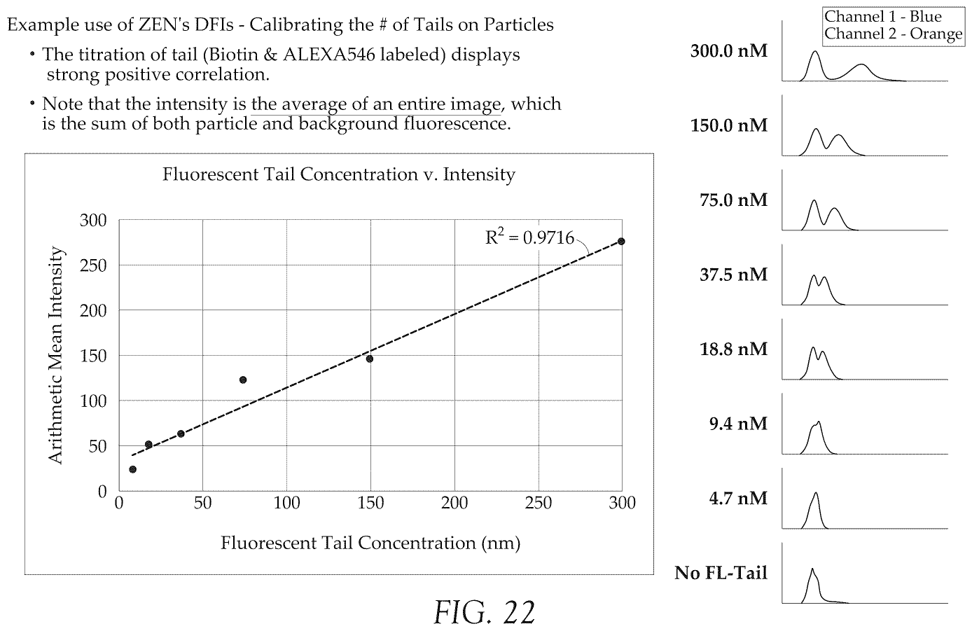

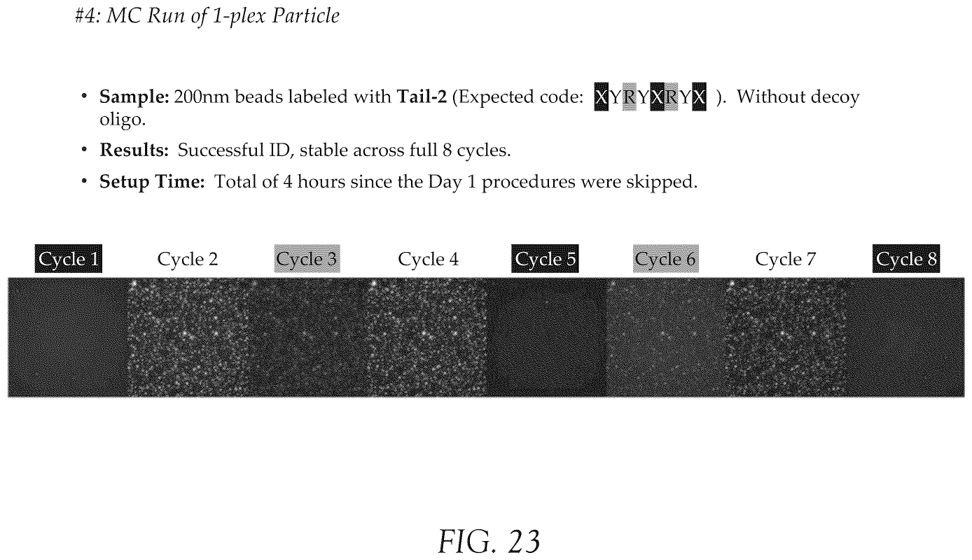

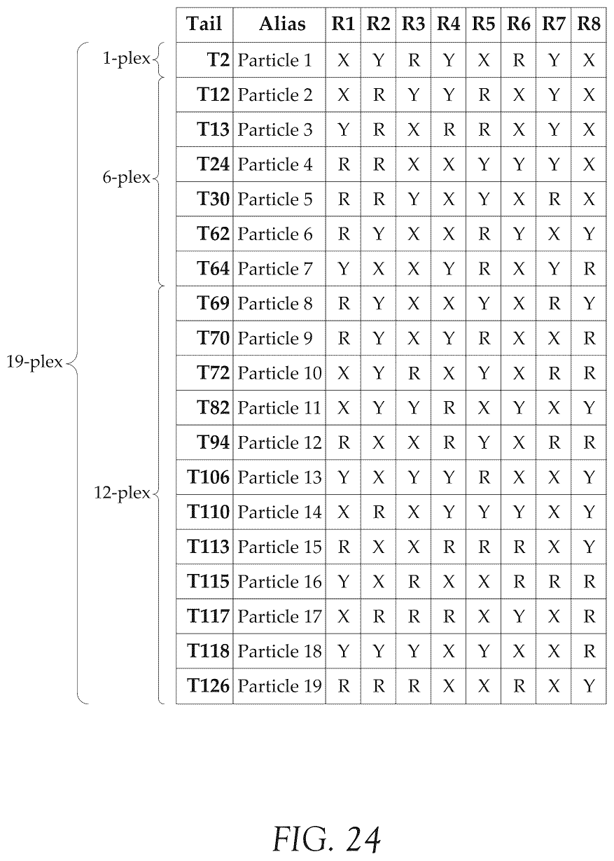

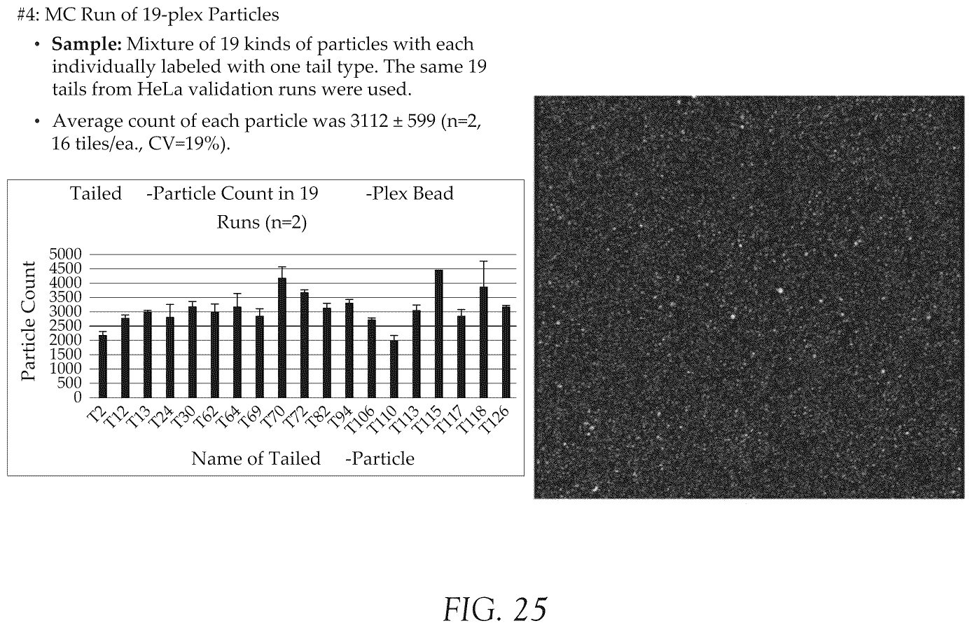

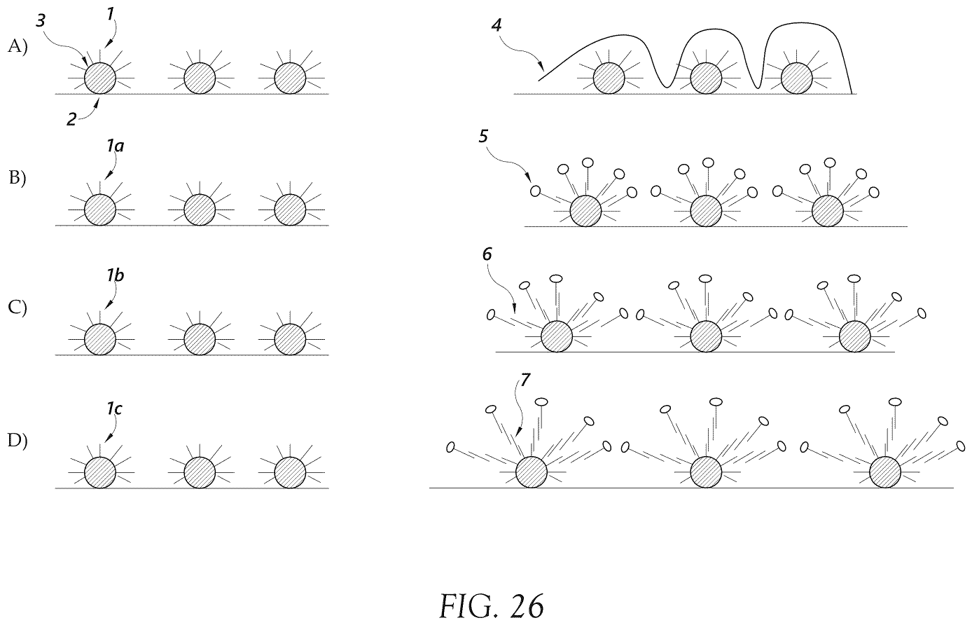

In certain embodiments, this pattern of binding or hybridization of the decoding oligonucleotides may be converted into a “codeword.” For example, the codewords could be also “101” and “110” for an analyte, where a value of 1 represents binding and a value of 0 represents no binding. The codewords may also have longer lengths in other embodiments (see FIG. 13). A codeword can be directly related to a specific unique identifier sequence of a analyte-specific probe. Accordingly, different analyte-specific probe may match certain codewords, which can then be used to identify the different analytes of the analyte-specific probe based on the binding patterns of the decoding oligonucleotide. However, if no binding is evident, then the codeword would be “000” in this example.

The values in each codeword can also be assigned in different fashions in some embodiments. For example, a value of 0 could represent binding while a value of 1 represents no binding. Similarly, a value of 1 could represent binding of a secondary nucleic acid probe with one type of signaling entity while a value of 0 could represent binding of a secondary nucleic acid probe with another type of distinguishable signaling entity. These signaling entities could be distinguished, for example, via different colors of fluorescence. In some cases, values in codewords need not be confined to 0 and 1. The values could also be drawn from larger alphabets, such as ternary (e.g., 0, 1, and 2) or quaternary (e.g., 0, 1, 2, and 3) systems. Each different value could, for example, be represented by a different distinguishable signaling entity, including (in some cases) one value that may be represented by the absence of signal.

The codewords for each analyte may be assigned sequentially, or may be assigned at random. For instance, a first analyte may be assigned to 101, while a second nucleic acid target may be assigned to 110. In addition, in some embodiments, the codewords may be assigned using an error-detection system or an error-correcting system, such as a Hamming system, a Golay code, or an extended Hamming system (or a SECDED system, i.e., single error correction, double error detection).

Generally speaking, such systems can be used to identify where errors have occurred, and in some cases, such systems can also be used to correct the errors and determine what the correct codeword should have been. For example, a codeword such as 001 may be detected as invalid and corrected using such a system to 101, e.g., if 001 is not previously assigned to a different target sequence. A variety of different error-correcting codes can be used, many of which have previously been developed for use within the computer industry; however, such error-correcting systems have not typically been used within biological systems. Additional examples of such error-correcting codes are discussed in more detail below.

“Essentially complementary” means, when referring to two nucleotide sequences, that both sequences can specifically hybridize to each other under stringent conditions, thereby forming a hybrid nucleic acid molecule with a sense and an antisense strand connected to each other via hydrogen bonds (Watson-and-Crick base pairs). “Essentially complementary” includes not only perfect base-pairing along the entire strands, i.e., perfect complementary sequences but also imperfect complementary sequences which, however, still have the capability to hybridize to each other under stringent conditions, Among experts it is well accepted that an “essentially complementary” sequence has at least 88% sequence identity to a fully or perfectly complementary sequence.

“Percent sequence identity” or “percent identity” in turn means that a sequence is compared to a claimed or described sequence after alignment of the sequence to be compared (the “Compared Sequence”) with the described or claimed sequence (the “Reference Sequence”). The percent identity is then determined according to the following formula: percent identity=100 [1−(C/R)] wherein C is the number of differences between the Reference Sequence and the Compared Sequence over the length of alignment between the Reference Sequence and the Compared Sequence, wherein
(i) each base or amino acid in the Reference Sequence that does not have a corresponding aligned base or amino acid in the Compared Sequence and
(ii) each gap in the Reference Sequence and
(iii) each aligned base or amino acid in the Reference Sequence that is different from an aligned base or amino acid in the Compared Sequence, constitutes a difference and (iiii) the alignment has to start at position 1 of the aligned sequences;
and R is the number of bases or amino acids in the Reference Sequence over the length of the alignment with the Compared Sequence with any gap created in the Reference Sequence also being counted as a base or amino acid.

If an alignment exists between the Compared Sequence and the Reference Sequence for which the percent identity as calculated above is about equal to or greater than a specified minimum Percent Identity then the Compared Sequence has the specified minimum percent identity to the Reference Sequence even though alignments may exist in which the herein above calculated percent identity is less than the specified percent identity.

In the “incubation” steps as understood herein the respective moieties or subjects such as probes or oligonucleotide, are brought into contact with each other under conditions well known to the skilled person allowing a specific binding or hybridization reaction, e.g. pH, temperature, salt conditions etc. Such steps may therefore, be preferably carried out in a liquid environment such as a buffer system which is well known in the art.

The “removing” steps according to the disclosure may include the washing away of the moieties or subjects to be removed such as the probes or oligonucleotides by certain conditions, e.g. pH, temperature, salt conditions etc., as known in the art.

It is understood that in an embodiment of the method according to the present disclosure a plurality of analytes can be encoded in parallel. This requires the use of different sets of analyte-specific probes in step (1). The analyte-specific probes of a particular set differ from the analyte-specific probes of another set. This means that the analyte-specific probes of set 1 bind to analyte 1, the analyte-specific probes of set 2 binds to analyte 2, the analyte-specific probes of set 3 binds to analyte 3, etc. In this embodiment also the use of different sets of decoding oligonucleotides is required in the methods according to the present disclosure.

The decoding oligonucleotides of a particular set differ from the decoding oligonucleotides of another set. This means, the decoding oligonucleotides of set 1 bind to the analyte-specific probes of above set 1 of analyte-specific probes, the decoding oligonucleotides of set 2 binds to the analyte-specific probes of above set 2 of analyte-specific probes, the decoding oligonucleotides of set 3 bind to the analyte-specific probes of above set 3 of analyte-specific probes, etc.

It is also understood that in an embodiment of the method according to the present disclosure a plurality of monitoring probes can be encoded in parallel. This requires the use of different sets of monitoring probes in step (2). The monitoring probes of a particular set differ from the monitoring probes of another set. This means that the monitoring probes of set 1 bind to decoding element 1, the monitoring probes of set 2 binds to decoding element 2, the monitoring probes of set 3 binds to decoding element 3, etc. In this embodiment also the use of different sets of decoding oligonucleotides is required in the methods according to the present disclosure.

The decoding oligonucleotides of a particular set differ from the decoding oligonucleotides of another set. This means, the decoding oligonucleotides of set 1 bind to the monitoring probes of above set 1 of monitoring probes, the decoding oligonucleotides of set 2 binds to the monitoring probes of above set 2 of monitoring probes, the decoding oligonucleotides of set 3 bind to the monitoring probes of above set 3 of monitoring probes, etc.

In this embodiment, where a plurality of analytes is to be encoded in parallel, the different sets of analyte-specific probes and/or monitoring probes may be provided as a premixture of different sets of analyte-specific probes and/or monitoring probes; and/or the different sets of decoding oligonucleotides may be provided as a premixture of different sets of decoding oligonucleotides. Each mixture may be contained in a single vial. Alternatively, the different sets of analyte-specific probes and/or monitoring probes; and/or the different sets of decoding oligonucleotides may be provided in steps singularly.

A “kit” is a combination of individual elements useful for carrying out the use and/or method of the disclosure, wherein the elements are optimized for use together in the methods. The kits may also contain additional reagents, chemicals, buffers, reaction vials etc. which may be useful for carrying out the method according to the disclosure. Such kits unify all essential elements required to work the method according to the disclosure, thus minimizing the risk of errors. Therefore, such kits also allow semi-skilled laboratory staff to perform the method according to the present disclosure.

The term “quencher” or “quencher dye” or “quencher molecule” refers to a dye or an equivalent molecule, such as nucleoside guanosine (G) or 2′-deoxyguanosine (dG), which is capable of reducing the fluorescence of a fluorescent reporter dye or donor dye. A quencher dye may be a fluorescent dye or non-fluorescent dye. When the quencher is a fluorescent dye, its fluorescence wavelength is typically substantially different from that of the reporter dye and the quencher fluorescence is usually not monitored during an assay. Some embodiments of the present disclosure disclose signal oligonucleotides comprising a quencher and/or a quencher in combination with a signal element (see FIG. 14), and therefore the signal oligonucleotides is not detectable during imaging.

In an embodiment of the disclosure the sample is a biological sample, preferably comprising biological tissue, further preferably comprising biological cells. A biological sample may be derived from an organ, organoids, cell cultures, stem cells, cell suspensions, primary cells, samples infected by viruses, bacteria or fungi, eukaryotic or prokaryotic samples, smears, disease samples, a tissue section.

The method is particularly qualified to monitor, encode, identify, detect, count, or quantify the in situ analysis of analytes or single analyte molecules in a biological sample, i.e., such as a sample which contains nucleic acids or proteins as said analytes. It is understood that the biological sample may be in a form as it is in its natural environment (i.e., liquid, semi-liquid, solid etc.), or processed, e.g., as a dried film on the surface of a device which may be re-liquefied before the method is carried out.

In another embodiment of the disclosure prior to step (2) the biological tissue and/or biological cells are fixed. For example, in some embodiments, the cell and/or the tissue is fixed prior to introducing the probes, e.g., to preserve the positions of the analytes like nucleic acids within the cell. Techniques for fixing cells are known to those of ordinary skill in the art. As non-limiting examples, a cell may be fixed using chemicals such as formaldehyde, paraformaldehyde, glutaraldehyde, ethanol, methanol, acetone, acetic acid, or the like. In one embodiment, a cell may be fixed using Hepes-glutamic acid buffer-mediated organic solvent (HOPE).

This measure has the advantage that the analytes to be encoded, e.g., the nuclei acids or proteins, are immobilized and cannot escape. In doing so, the analytes then prepared for a better detection or encoding by the method according to the disclosure.

In yet a further embodiment within the set of analyte-specific probes the individual analyte-specific probes comprise binding elements (S1, S2, S3, S4, S5) which specifically interact with different sub-structures of one of the analytes to be encoded.

By this measure the method becomes even more robust and reliable because the signal intensity obtained at the end of the method or a cycle, respectively, is increased. It is understood that the individual probes of a set while binding to the same analyte differ in their binding position or binding site at or on the analyte. The binding elements S1, S2, S3, S4, S5 etc. of the first, second, third fourth, fifth etc. analyte-specific probes therefore bind to or at a different position which, however, may or may not overlap.

In an advantageous embodiment, the present disclosure pertains to kit for multiplex analyte encoding, comprising:
(A1) at least a first set of analyte-specific probes for encoding different analytes, each set of analyte-specific probes interacting with a different analyte, wherein if the analyte is a nucleic acid each set of analyte-specific probes comprises analyte-specific probes which specifically interact with different sub-structures of the same analyte, each analyte-specific probe comprising
   (aa) a binding element(S) that specifically interacts with one of the different analytes to be encoded, and
   (bb) an identifier element (T) comprising a nucleotide sequence which is unique to the analyte to be encoded (unique identifier sequence),
and
(A2) at least a second set of analyte-specific probes for encoding different analytes, each set of analyte-specific probes interacting with a different analyte, wherein if the analyte is a nucleic acid each set of analyte-specific probes comprises analyte-specific probes which specifically interact with different sub-structures of the same analyte, each analyte-specific probe comprising
   (aa) a binding element(S) that specifically interacts with one of the different analytes to be encoded, and
   (bb) an identifier element (T) comprising a nucleotide sequence which is unique to the analyte to be encoded (unique identifier sequence),
wherein the analyte-specific probes of a particular set of analyte-specific probes differ from the analyte-specific probes of another set of analyte-specific probes in the nucleotide sequence of the identifier element (T), and
wherein the analyte-specific probes in each set of analyte-specific probes binds to the same analyte and comprises the same nucleotide sequence of the identifier element (T) which is unique to said analyte; and
wherein (optionally) the number of probes and/or targets of first set of analyte-specific probes according to step A1 (i.e. the transcript plexity of A1) is at least 10 times higher than the number of probes and/or targets of the second set of analyte-specific probes according to step A2 (i.e. the transcript plexity of A2); and
(B1) at least a first set of decoding oligonucleotides per analyte, wherein in each set of decoding oligonucleotides for an individual analyte each decoding oligonucleotide comprises:
   (aa) an identifier connector element (t) comprising a nucleotide sequence which is essentially complementary to at least a section of the unique identifier sequence of the identifier element (T) of the corresponding analyte-specific probe set, and
   (bb) a translator element (c) comprising a nucleotide sequence allowing a specific hybridization of a signal oligonucleotide; and
(B2) at least a second set of decoding oligonucleotides per analyte, wherein in each set of decoding oligonucleotides for an individual analyte each decoding oligonucleotide comprises:
   (aa) an identifier connector element (t) comprising a nucleotide sequence which is essentially complementary to at least a section of the unique identifier sequence of the identifier element (T) of the corresponding analyte-specific probe set, and
   (bb) a translator element (c) comprising a nucleotide sequence allowing a specific hybridization of a signal oligonucleotide;
wherein the decoding oligonucleotides of a set for an individual analyte differ from the decoding oligonucleotides of another set for a different analyte in the identifier connect element (t); and
(C) a set of signal oligonucleotides, each signal oligonucleotide comprising:
   (aa) a translator connector element (C) comprising a nucleotide sequence which is essentially complementary to at least a section of the nucleotide sequence of a translator element (c) comprised in a decoding oligonucleotide, and
   (bb) a signal element;
   And
   further comprising a monitoring probe comprising at least an identifier element (t) comprising a nucleotide sequence which is essentially complementary to at least a section of the unique set identifier sequence of the identifier element (T) of the corresponding binding element.

A multiplex method or assay allow the simultaneously measurement of multiple analytes according to the present disclosure it may be used to determine the presence or absence of a plurality of predetermined (known) analytes like nucleic acid target sequences in a sample. An analyte may be “predetermined” in that its sequence is known to design a probe that binds to the that target.

In some advantageous embodiments according to the present disclosure at least 2, at least 5, at least 10, at least 15, at least 20, in particular at least 25, in particular at least 30 different analytes are detected and/or quantified in a sample in parallel. For example, there may be at least 5, at least 10, at least 20, at least 50, at least 75, at least 100, at least 300, at least 1,000, at least 3,000, at least 10,000, or at least 30,000 distinguishable analyte-specific probes that are applied to a sample, e.g., simultaneously or sequentially.

In the multiplexing methods of the present disclosure, in particular at least 2 different subgroups of analytes (e.g., mRNA molecules) i.e., tags with spatially overlap (i.e. a distance beyond the diffraction limit of the respective microscope) are targeted.

In some advantageous embodiments, at least 4 rounds to collect information for identification of the analyte are carried out, wherein multiple readout increases the accuracy of identification and avoids false positives. The unique tag can be identified by various techniques, including hybridization, e.g., with labelled probes, directly or indirectly or by sequencing (by synthesis, ligation). In particular, the identity of the tag can be encoded with one single signal (binary code), two or more signals, wherein the signal can be a fluorescent label (e.g. attached to an oligonucleotide).

In some advantageous embodiments according to the present disclosure, the kit does not comprise sets of analyte-specific probes as defined under item A1) and A2).

Preferably, if the analyte in the kits or methods according to the present disclosure is a nucleic acid, each set of analyte-specific probes comprises at least five (10) analyte-specific probes, in particular at least fifteen (15) analyte-specific probes, in particular at least twenty (20) analyte-specific probes which specifically interact with different sub-structures of the same analyte. Nucleic acid analyte includes specific DNA molecules, e.g. genomic DNA, nuclear DNA, mitochondrial DNA, viral DNA, bacterial DNA, extra- or intracellular DNA etc., and specific mRNA molecules, e.g. hnRNA, miRNA, viral RNA, bacterial RNA, extra- or intracellular RNA, etc.,

Preferably, if the analyte in the kits or methods according to the present disclosure is a peptide, a polypeptide or a protein, each set of analyte-specific probes comprises at least one, preferably at least two (2) analyte-specific probes, in particular at least three (3) analyte-specific probes, in particular at least four (4) analyte-specific probes which specifically interact with different sub-structures of the same analyte.

In some advantageous embodiments according to the present disclosure the kit comprises at least one, preferably at least two different sets of signal oligonucleotides, wherein the signal oligonucleotides in each set comprise a different signal element and comprise a different connector element (C).

In particular, the kit may comprise at least one, preferably at least two different sets of decoding oligonucleotides per analyte, wherein the decoding oligonucleotides comprised in these different sets comprise the same identifier connector element (t) comprising a nucleotide sequence which is essentially complementary to at least a section of the unique identifier sequence of the identifier element (T) of the corresponding analyte-specific probe set, and wherein the decoding oligonucleotides of the different sets per analyte differ in the translator element (c) comprising a nucleotide sequence allowing a specific hybridization of a signal oligonucleotide.

In some embodiments the kit comprises at least one, preferably at least two different sets of decoding oligonucleotides per analyte, wherein the decoding oligonucleotides comprised in these different sets comprise the same identifier connector element (t) comprising a nucleotide sequence which is essentially complementary to at least a section of the unique identifier sequence of the identifier element (T) of the corresponding analyte-specific probe set, and wherein the decoding oligonucleotides of the different sets for at least one analyte differ in the translator element (c) comprising a nucleotide sequence allowing a specific hybridization of a signal oligonucleotide.

In some advantageous embodiments, the number of different sets of decoding oligonucleotides per analyte comprising different translator elements (c) corresponds to the number of different sets of signal oligonucleotides comprising different connector elements (C). However, the decoding oligonucleotides in a particular set of decoding oligonucleotides may interacts with identical identifier elements (T) which are unique to a particular analyte. In particular, all sets of decoding oligonucleotides for the different analytes may comprise the same type(s) of translator element(s) (c).

In another embodiment, the present disclosure is generally directed to a methods including acts of exposing a sample to a plurality of analyte-specific probes; for each of the analyte-specific probes, determining binding of the analyte-specific probes within the sample; creating codewords based on the binding of the analyte-specific probes, the decoding oligonucleotides and the signal oligonucleotides; and for at least some of the codewords, matching the codeword to a valid codeword. In certain embodiments, this pattern of binding or hybridization of the analyte-specific probes, the decoding oligonucleotides and the signal oligonucleotides may be converted into a “codeword.” For example, for instance, the codewords may be “101” and “110” for a first analyte and a second analyte, respectively, where a value of 1 represents binding and a value of 0 represents no binding of decoding oligonucleotides and/or the binding of signal oligonucleotides without and/or quenched signal element. The analyte in the detection round/cycle is therefore not detectable during imaging.

To create such a zero (0) in a codeword for an individual analyte the kit may comprise:
(D) at least a set of non-signal decoding oligonucleotides for binding to a particular identifier element (T) of analyte-specific probes, wherein the decoding oligonucleotides in the same set of non-signal decoding oligonucleotides interacting with the same different identifier element (T), wherein each non-signal decoding oligonucleotide comprises an identifier connector element (t) comprising a nucleotide sequence which is essentially complementary to at least a section of a unique identifier sequence, and does not comprise a translator element (c) comprising a nucleotide sequence allowing a specific hybridization of a signal oligonucleotide.

To create such a zero (0) in a codeword for an individual analyte the kit may comprise:
(D) at least a set of non-signal decoding oligonucleotides for binding to a particular identifier element (T) of analyte-specific probes, wherein the decoding oligonucleotides in the same set of non-signal decoding oligonucleotides interacting with the same different identifier element (T), wherein each non-signal decoding oligonucleotide comprises an identifier connector element (t) comprising a nucleotide sequence which is essentially complementary to at least a section of a unique identifier sequence, and comprise a translator element that does not interact/bind to a signal oligonucleotide due to an instabil binding sequence and/or due to the translator element is to short (c) comprising a nucleotide sequence allowing a specific hybridization of a signal oligonucleotide.

In some advantageous embodiments, the kit comprises:
(D) at least one, preferably at least two (2) different sets of non-signal decoding oligonucleotides for binding to at least one, preferably at least two different identifier elements (T) of analyte-specific probes, each set of non-signal decoding oligonucleotides interacting with a different identifier element (T),
wherein each non-signal decoding oligonucleotide comprises an identifier connector element (t) comprising a nucleotide sequence which is essentially complementary to at least a section of a unique identifier sequence, and does not comprise a translator element (c) comprising a nucleotide sequence allowing a specific hybridization of a signal oligonucleotide.

In some advantageous embodiments, the different sets of non-signal decoding oligonucleotides may be comprised in a pre-mixture of different sets of non-signal decoding oligonucleotides or exist separately.

Furthermore, in some advantageous embodiments the kit may comprise:
(E) a set of non-signal oligonucleotides, each non-signal oligonucleotide comprising:
(aa) a translator connector element (C) comprising a nucleotide sequence which is essentially complementary to at least a section of the nucleotide sequence of the translator element (c), and (bb) a quencher (Q), a signal element and a quencher (Q), or does not comprise a signal element.

In some advantageous embodiments, the kit comprises:
(E) at least one, preferably at least two sets of non-signal oligonucleotides, each non-signal oligonucleotide comprising:
(aa) a translator connector element (C) comprising a nucleotide sequence which is essentially complementary to at least a section of the nucleotide sequence of the translator element (c), and
(bb) a quencher (Q), a signal element and a quencher (Q), or does not comprise a signal element.

In some advantageous embodiments, the different sets of non-signal oligonucleotides may be comprised in a pre-mixture of different sets of non-signal oligonucleotides or exist separately.

Further, in some embodiments the decoding oligonucleotides in a particular set of decoding oligonucleotides interacts with identical identifier elements (T) which are unique to a particular analyte.

In some advantageous embodiments, the different sets of decoding oligonucleotides may be comprised in a pre-mixture of different sets of decoding oligonucleotides or exist separately. In some advantageous embodiments, the different sets of analyte-specific probes may be comprised in a pre-mixture of different sets of analyte-specific probes or exist separately. In some advantageous embodiments, the different sets of signal oligonucleotides may be comprised in a pre-mixture of different sets of signal oligonucleotides or exist separately.

In some advantageous embodiments, a mixture of decoding oligonucleotides and/or multi-decoders is provided that specifically hybridize to the unique identifier sequences of the probe sets. In some embodiments, the decoding oligonucleotides comprise of at least one, preferably at least two sequence elements, a first element that is complementary to the unique identifier sequences of the corresponding probe set and a second sequence element (translator element) that provides a sequence for the specific hybridization of a signal oligonucleotide, the translator element defines the type of signal that is recruited to the decoding oligonucleotide. In some embodiments multi-decoders comprising at least three sequence elements are used, a first element that is complementary to the unique identifier sequences of the corresponding probe set and at least to additional sequence elements (translator elements) that provide sequences for the specific hybridization of at least one, preferably at least two different signal oligonucleotides. The translator elements define the type of signals that are recruited to the multi-decoder. Different possible structures of a multi-decoder can be seen in FIG. 15. Since a multi-decoder does recruit a full signal oligonucleotide per translator element, the brightness of the signals in each channel is not lower than the brightness of signals with decoding oligonucleotides.

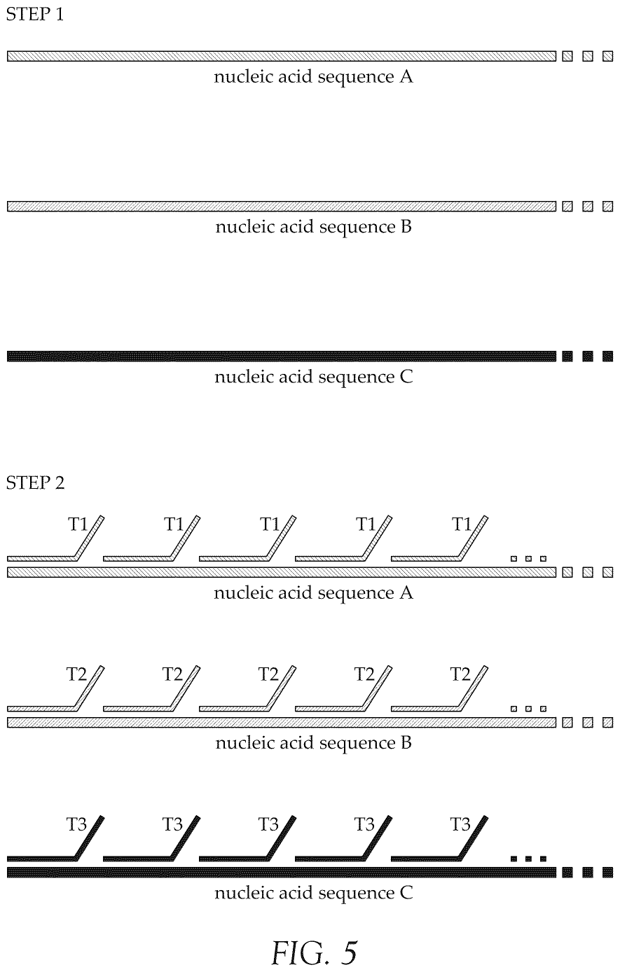

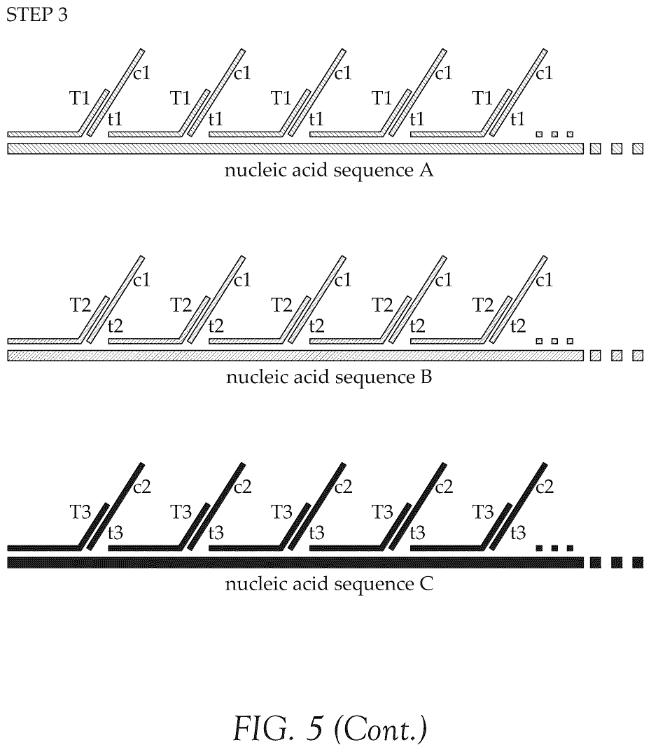

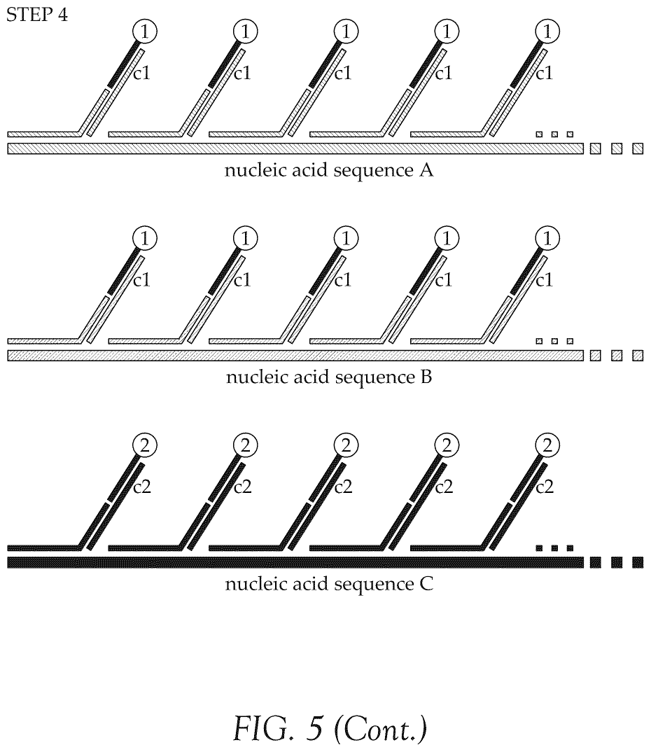

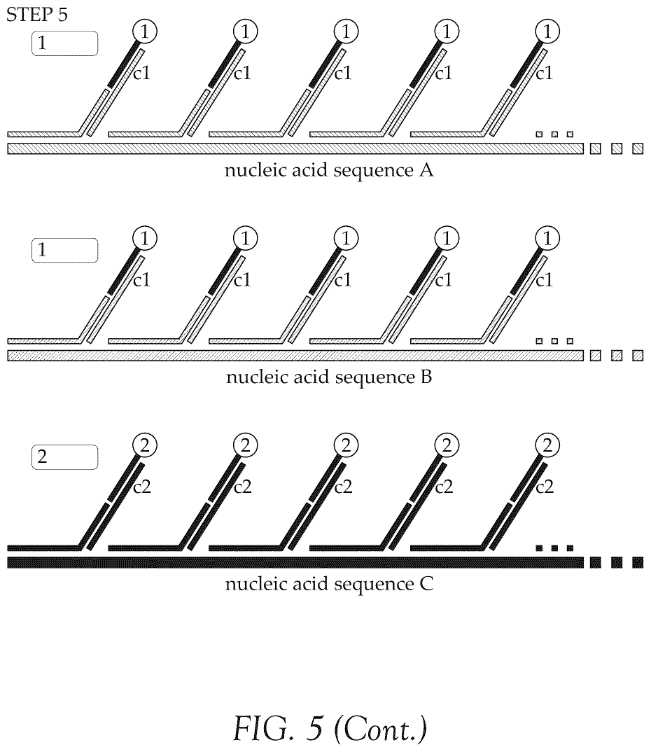

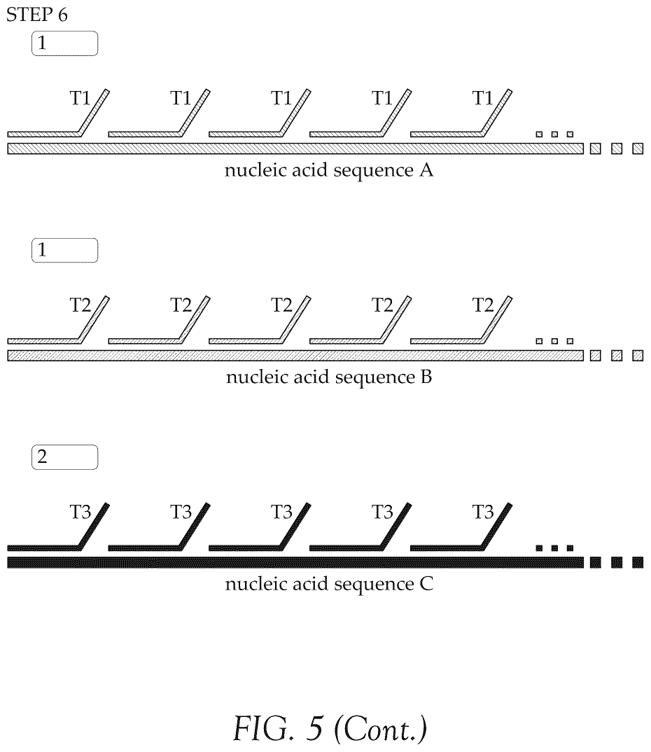

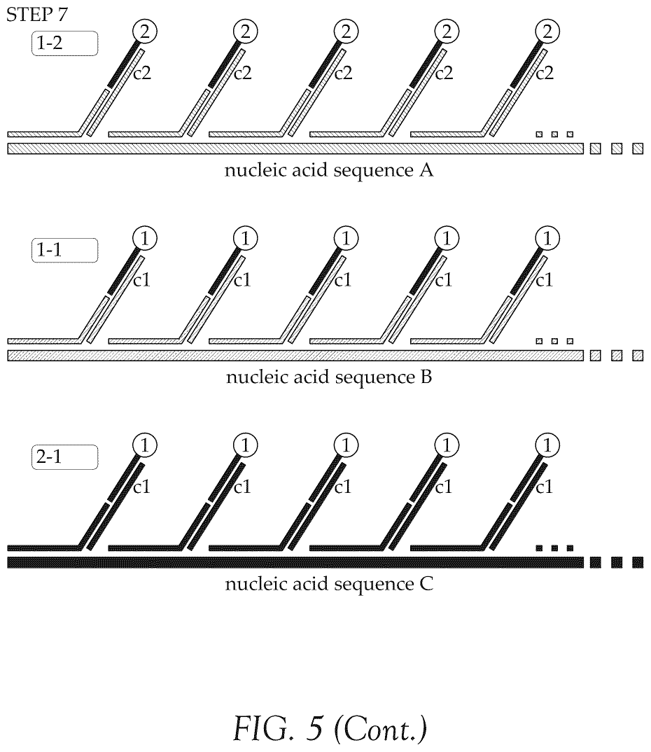

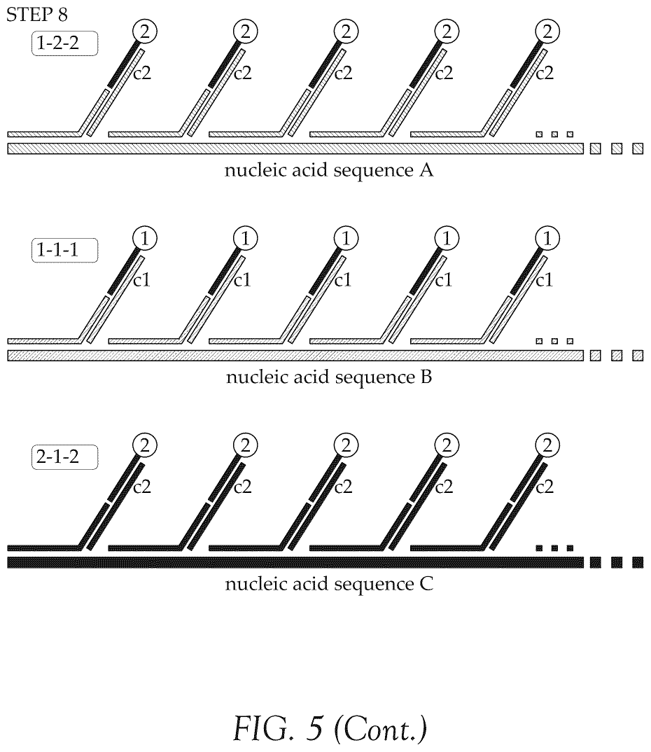

The usage of multi-decoders increases further the efficiency of the encoding scheme. FIG. 16 shows a possible encoding scheme using multi-decoders based upon the same conditions used for the examples with the decoding oligonucleotide with two sequence elements. One can clearly see that the multi-decoder based encoding scheme can create a higher hamming distance, with the same number of rounds and the same number of different signal oligonucleotides used in the example of FIG. 5.

As mentioned above the analyte to be encoded may be a nucleic acid, preferably DNA, PNA or RNA, in particular mRNA, a peptide, polypeptide, a protein, and/or mixtures thereof.

In some advantageous embodiments, the binding element(S) comprises an amino acid sequence allowing a specific binding to the analyte to be encoded. The binding element(S) may comprise moieties which are affinity moieties from affinity substances or affinity substances in their entirety selected from the group consisting of antibodies, antibody fragments, anticalin proteins, receptor ligands, enzyme substrates, lectins, cytokines, lymphokines, interleukins, angiogenic or virulence factors, allergens, peptidic allergens, recombinant allergens, allergen-idiotypical antibodies, autoimmune-provoking structures, tissue-rejection-inducing structures, immunoglobulin constant regions and combinations thereof.

In some advantageous embodiments, the binding element (S) may comprise or is an antibody or an antibody fragment selected from the group consisting of Fab, scFv; single domain, or a fragment thereof, bis scFv, F(ab)2, F(ab)3, minibody, diabody, triabody, tetrabody and tandab.

As mentioned above, the method according to the present disclosure comprises selectively removing the decoding oligonucleotides and signal oligonucleotides from the sample, thereby essentially maintaining the specific binding of the analyte-specific probes to the analyte to be encoded. In particular all steps are performed sequentially. However, some steps may be performed simultaneously, in particular the contacting steps A) to C), in particular B) and C).

By this measure the requirements for another round/cycle of binding further decoding oligonucleotides to the same analyte-specific probes are established, thus finally resulting in a code or encoding scheme comprising more than one signal. This step is realized by applying conditions and factors well known to the skilled person, e.g., pH, temperature, salt conditions, oligonucleotide concentration, polymers etc.

In some advantageous embodiments, all steps are automated, in particular by using a robotic system and/or an optical multiplexing system according to the present disclosure. In some examples, the steps may be performed in a fluidic system.

As mentioned above, with the methods according to the present disclosure an encoding scheme with a code word per analyte set is generated. Therefore, each analyte set may be associated with a specific code word, wherein said code word comprise a number of positions, and wherein each position corresponds to one cycle resulting in a plurality of distinguishable encoding schemes with the plurality of code words. In particular, said encoding scheme may be predetermined and allocated to the analyte to be encoded.

In some advantageous embodiments, the code words obtained for the individual analytes in the performed cycles comprise the detected signals and additionally at least one element corresponding to no detected signal like 0,1 or 0, 1, 2 etc. (see also FIG. 13 and FIG. 14). In particular, no signal is detected for at least one analyte within at least one cycle if using the non-signal probe according to FIG. 14, Nr. 2 to 4, or a non-signal decoding oligonucleotide as shown in FIG. 14 Nr. 5, or if in one cycle no decoding oligonucleotide is contacted with the corresponding identifier sequence comprised on analyte-specific probe interacting with the corresponding analyte in the sample. In this cycle the position has the value zero (0).

In some advantageous embodiments, at least for one individual analyte a position of the code word is zero (0). In particular, the code word zero (0) is generated by using no decoding oligonucleotides having an identifier connector element (t) comprising a nucleotide sequence which is essentially complementary to at least a section of the unique identifier sequence of the identifier element (T) of a corresponding analyte-specific probe for an individual analyte. As mentioned above, in some embodiments, if at least for one individual analyte a position of the code word is zero (0) in this cycle no corresponding decoding oligonucleotides having an identifier connector element (t) comprising a nucleotide sequence which is essentially complementary to at least a section of the unique identifier sequence of the identifier element (T) of a corresponding analyte-specific probe for an individual analyte are used.

Furthermore, in some advantageous embodiments the sample is contacted with at least one, preferably at least two different sets of signal oligonucleotides, wherein the signal oligonucleotides in each set comprise a different signal element and comprise a different connector element (C).

In more particular embodiments, the sample is contacted with at least one, preferably at least one, preferably at least two different sets of decoding oligonucleotides per analyte, wherein the decoding oligonucleotides comprised in these different sets comprise the same identifier connector element (t) comprising a nucleotide sequence which is essentially complementary to at least a section of the unique identifier sequence of the identifier element (T) of the corresponding analyte-specific probe set, and wherein the decoding oligonucleotides of the different sets per analyte differ in the translator element (c) comprising a nucleotide sequence allowing a specific hybridization of a signal oligonucleotide and a monitoring probe comprising at least an identifier element (t) comprising a nucleotide sequence which is essentially complementary to at least a section of the unique set identifier sequence of the identifier element (T) of the corresponding binding element; wherein the monitoring probe is added before, during and/or after adding the decoding element.

As mentioned above, the different sets of non-signal oligonucleotides may be comprised in a pre-mixture of different sets of non-signal oligonucleotides or exist separately.

In further embodiments, the decoding oligonucleotides in a particular set of decoding oligonucleotides interacts with identical identifier elements (T) which are unique to a particular analyte.

As mentioned above, the different sets of decoding oligonucleotides may be comprised in a pre-mixture of different sets of decoding oligonucleotides or exist separately as well as the different sets of analyte-specific probes may be comprised in a pre-mixture of different sets of analyte-specific probes or exist separately as well the different sets of signal oligonucleotides may be comprised in a pre-mixture of different sets of signal oligonucleotides or exist separately. In some advantageous embodiments of the method according to the present disclosure, the binding element(S) comprise a nucleic acid comprising a nucleotide sequence allowing a specific binding to the analyte to be encoded, preferably a specific hybridization to the analyte to be encoded.

In some advantageous embodiments of the method according to the present disclosure, after step A) and before step B) the non-bound analyte-specific probes may be removed, in particular by washing, further after step B) and before step C) the non-bound decoding oligonucleotides may be removed, in particular by washing further, after step C) and before step D) the non-bound signal oligonucleotides may be removed, in particular by washing.

In some advantageous embodiments of the method according to the present disclosure, the analyte specific probes may be incubated with the sample, thereby allowing a specific binding of the analyte specific probes to the analytes to be encoded, further the decoding oligonucleotides may be incubated with the sample, thereby allowing a specific hybridization of the decoding oligonucleotides to identifier elements (T) of the respective analyte-specific probes, further the signal oligonucleotides may be incubated with the sample, thereby allowing a specific hybridization of the signal oligonucleotides to translator elements (T) of the respective decoding oligonucleotides.

As mentioned above, the analyte to be encoded may be a nucleic acid, preferably DNA, PNA, RNA, in particular mRNA, a peptide, polypeptide, a protein or combinations thereof. Therefore, the binding element(S) may comprise an amino acid sequence allowing a specific binding to the analyte to be encoded. Examples for a binding element(S) are moieties which are affinity moieties from affinity substances or affinity substances in their entirety selected from the group consisting of antibodies, antibody fragments, anticalin proteins, receptor ligands, enzyme substrates, lectins, cytokines, lymphokines, interleukins, angiogenic or virulence factors, allergens, peptidic allergens, recombinant allergens, allergen-idiotypical antibodies, autoimmune-provoking structures, tissue-rejection-inducing structures, immunoglobulin constant regions and combinations thereof. In particular, the binding element(S) is an antibody or an antibody fragment selected from the group consisting of Fab, scFv; single domain, or a fragment thereof, bis scFv, Fab 2, Fab 3, minibody, diabody, triabody, tetrabody and tandab.

By this measure the method is further developed to such an extent that the encoded analytes can be detected by any means which is adapted to visualize the signal element. Examples of detectable physical features include e.g., light, chemical reactions, molecular mass, radioactivity, etc.

In some advantageous embodiments, the signal caused by the signal element, therefore in particular the binding of the signal oligonucleotides to the decoding oligonucleotides, interacting with the corresponding analyte probes, bound to the respective analyte is determined by:
(a) Imaging at least a portion of the sample; and/or
(b) Using an optical imaging technique; and/or
(c) Using a fluorescence imaging technique; and/or
(d) Multi-color fluorescence imaging technique; and/or
(e) Super-resolution fluorescence imaging technique.

The kits and method according to the present disclosure may be used ideally for in vitro methods for diagnosis of a disease selected from the group comprising cancer, neuronal diseases, cardiovascular diseases, inflammatory diseases, autoimmune diseases, diseases due to a viral or bacterial infection, skin diseases, skeletal muscle diseases, dental diseases, and prenatal diseases.

Further, the kits and method according to the present disclosure may be used also ideally for in vitro methods for diagnosis of a disease in plants selected from the group comprising: diseases caused by biotic stress, preferably by infectious and/or parasitic origin, or diseases caused by abiotic stress, preferably caused by nutritional deficiencies and/or unfavorable environment.

Further, the kits and method according to the present disclosure may be used also ideally for in vitro methods for screening, identifying and/or testing a substance and/or drug comprising:
(a) contacting a test sample comprising a sample with a substance and/or drug
(b) detecting different analytes in a sample by sequential signal-encoding of said analytes with a method according to the present disclosure.

An optical multiplexing system suitable for the method according to the present disclosure, comprising at least:
a reaction vessel for containing the kits or part of the kits according to the present disclosure;
a detection unit comprising a microscope, in particular a fluorescence microscope;
a camera;
a liquid handling device.

In some embodiments, optical multiplexing system may comprise further a heat and cooling device and/or a robotic system.

In some embodiments, the method according to the present disclosure encodes a nucleic acid analyte, such as an mRNA, e.g., such an mRNA coding for a particular protein.

In some advantageous embodiments, the method described herein is used for specific detection of many different analytes in parallel. The technology allows to distinguish a higher number of analytes than different signals are available. The process includes at least four consecutive rounds of specific binding, signal detection and selective denaturation (if a next round is required), eventually producing a signal code. To decouple the dependency between the analyte specific binding and the oligonucleotides providing the detectable signal, a so called “decoding”-oligonucleotide is introduced. The decoding oligonucleotide transcribes the information of the analyte specific probe set to the signal oligonucleotides.

In a specific embodiment the method may comprise the steps of 1. providing one or more analyte specific probe sets, the set of analyte specific probes consist of one or more different probes, each differing in the binding moiety that specifically interacts with the analyte, all probes of a single probe set are tethered to a sequence element (unique identifier), that is unique to a single probe set and allows the specific hybridization of a decoding oligonucleotide, 2. specific binding of the probe sets to their target binding sites of the analyte, 3. eliminating non-bound probes (e.g. by a wash step), 4. providing a mixture of decoding oligonucleotides that specifically hybridize to the unique identifier sequences of the probe sets, the decoding oligonucleotides comprise of at least one, preferably at least two sequence elements, a first element that is complementary to the unique identifier sequences of the corresponding probe set and a second sequence element (translator element) that provides a sequence for the specific hybridization of a signal oligonucleotide, the translator element defines the type of signal that is recruited to the decoding oligonucleotide, 5. specific hybridization of the decoding oligonucleotides to the unique identifier sequences provided by the bound probe sets, 6, eliminating non-bound decoding oligonucleotides (e.g. by washing step), 7. providing a mixture of signal oligonucleotides, comprising of a signal that can be detected and a nucleic acid sequence that specifically hybridizes to the translator element of one of the decoding oligonucleotides used in the former hybridization step, 8. specific hybridization of the signal oligonucleotides, 9. eliminating non-bound signal oligonucleotides, 10. detection of the signals, 11. selective release of decoding oligonucleotides and signal oligonucleotides while the binding of specific probe sets to the analyte is almost or completely unaffected, 12. eliminating released decoding oligonucleotide and signal oligonucleotides (e.g. by a washing step) while the binding of specific probes sets to the analytes is almost or completely unaffected, repeating the steps 4 to 12 at least three times until the detection of a sufficient number of signals to generate an encoding scheme for each different analyte of interest.

It is to be understood that the before-mentioned features and those to be mentioned in the following cannot only be used in the combination indicated in the respective case, but also in other combinations or in an isolated manner without departing from the scope of the disclosure.

The disclosure is now further explained by means of embodiments resulting in additional features, characteristics and advantages of the disclosure. The embodiments are of pure illustrative nature and do not limit the scope or range of the disclosure. The features mentioned in the specific embodiments are general features of the disclosure which are not only applicable in the specific embodiment but also in an isolated manner in the context of any embodiment of the disclosure.

The method disclosed herein is used for specific detection of many different analytes in parallel. The technology allows distinguishing a higher number of analytes than different signals are available. The process preferably includes at least one, preferably at least two consecutive rounds of specific binding, signal detection and selective denaturation (if a next round is required), eventually producing a signal code. To decouple the dependency between the analyte specific binding and the oligonucleotides providing the detectable signal, a so called “decoding” oligonucleotide is introduced. The decoding oligonucleotide transcribes the information of the respective analyte specific probe set to the signal oligonucleotides.

The present disclosure pertains further to monitor methods of detecting an analyte, comprising:
attaching a plurality of analyte-specific probes to the analyte, wherein the analyte-specific probes independently attach to the analyte and wherein the analyte-specific probes share a common identifier segment (T);
annealing a plurality of first decoding oligonucleotides to the analyte-specific probes, wherein the first decoding oligonucleotides share a first common region that is reverse complementary to the common identifier segment and a second common region;
annealing a first signal oligonucleotide to at least one of the plurality of first decoding oligonucleotides such that an oligo tethered to the first signal oligonucleotide is reverse complementary to the second common region;
detecting the first signal oligonucleotide;
removing the plurality of first decoding oligonucleotides;
annealing a plurality of second decoding oligonucleotides to the analyte-specific probes, wherein the second decoding oligonucleotides share a first common region that is reverse complementary to the common identifier segment and a second decoding oligonucleotides second common region that differs from the second common region of the first decoding;
annealing a second signal oligonucleotide to at least one of the plurality of second decoding oligonucleotides such that an oligo tethered to the signal oligonucleotide is reverse complementary to the second decoding oligonucleotide second common region; and detecting the second signal oligonucleotide.

In particular, in the above mentioned embodiment a second aliquot of a plurality of first decoding oligonucleotides is annealed to the analyte-specific probes. Furthermore, a first aliquot of a plurality of first decoding oligonucleotides is annealed to the analyte-specific probes.

In some further advantageous embodiments, the present disclosure pertains to a method of assigning an analyte to a position in an image, comprising assigning a fluorescence pattern to the analyte, observing the fluorescence pattern at the position in the image, and assigning the analyte to the position, in particular wherein observing the fluorescence pattern comprises repeating steps of labeling the position using a fluorophore tagged oligo drawn from a re-accessible pool, performing a single excitation at the position in the image, and contacting the analyte to a denaturant, in particular wherein observing the fluorescence pattern comprises repeating steps of labeling the position using a fluorophore tag-recruiting bridging oligo drawn from a re-accessible pool, performing a single excitation at the position in the image, and contacting the analyte to a denaturant.

In some further advantageous embodiments, the present disclosure pertains to a composition comprising a cell having nucleic acids distributed therein, wherein a first nucleic acid is tagged by a first plurality of probes that target adjacent segments of the first nucleic acid and that share a common first tether segment; a second nucleic acid is tagged by a second plurality of probes that target adjacent segments of the second nucleic acid and that share a common second tether segment; and a third nucleic acid is tagged by a third plurality of probes that target adjacent segments of the third nucleic acid and that share a common third tether segment; a first adapter population comprising molecules having a first tether reverse complementary region and a first fluorophore adapter tether; a second adapter population comprising molecules having a second tether reverse complementary region and a second fluorophore adapter tether; a third adapter population comprising molecules having a third tether reverse complementary region and a first fluorophore adapter tether; a population of first fluorophores having a first tether reverse complementary region; and a population of second fluorophores having a second tether reverse complementary region.

In some further advantageous embodiments, the present disclosure pertains to a method of assigning coded fluorescence patterns to a plurality of target analytes in a cell, comprising: subjecting the cell to a plurality of detection rounds, each detection round comprising: contacting the cell to representatives of the same at least one, preferably at least two populations of tagged fluorescence moieties, and removing the fluorescent moieties after a single excitation event, in particular
wherein the number of patterns detectable increases exponentially with the number of detection rounds,
wherein the fluorescence moieties are not tagged with nucleic acid tags that are specific to the target nucleic acids, and
wherein separate aliquots of common tagged fluorescence moieties are used across multiple detection rounds.

In particular, with the above notified method a total decoding efficiency of at least 30% may be achieved.

In some further advantageous embodiments, the present disclosure pertains to a method of assigning coded fluorescence patterns to a plurality of target analytes in a cell, comprising: contacting a target to a bipartite labeling probe, the bipartite labeling probe comprising a target-specific moiety and a fluorophore-specifying moiety; contacting the bipartite labeling probe to a first aliquot of a fluorophore reservoir comprising no more than two populations of fluorophores; replacing the fluorophore specifying moiety in the bipartite probe, and contacting the bipartite labeling probe to a second aliquot of the fluorophore reservoir comprising the same no more than two populations.

In some embodiments of the above notified method, replacing the fluorophore specifying moiety in the bipartite probe comprises denaturing a binding between a target-specific moiety and a fluorophore-specifying moiety after subjecting the bipartite labeling probe bound to a fluorophore of the fluorophore to excitation energy. In particular, replacing the fluorophore specifying moiety in the bipartite probe comprises drawing from one of no more than two fluorophore specifying moiety reservoirs,

In some further advantageous embodiments, the present disclosure pertains to monitoring a method of detecting an analyte, comprising:
attaching a plurality of probes to the analyte, in particular a nucleic acid, wherein the probes independently attach/anneal to the analyte and wherein the probes share a common identifier segment;
annealing a plurality of first adapter segments to the probes, wherein the first adapter segments share a first common region that is reverse complementary to the common identifier segment and a second common region, in particular configured to accommodate a single reporter/selected from no more than two reporter categories;
annealing a first reporter to at least one of the plurality of first adapter segments such that an oligo tethered to the first reporter is reverse complementary to the second common region; detecting the first reporter;
removing the plurality of first adapter segments, in particular without annealing a second reporter to the at least one of the plurality of first adapter segments;
annealing a plurality of second adapter segments to the probes, wherein the second adapter segments share a first common region that is reverse complementary to the common identifier segment and a second adapter second common region that differs from the second common region of the first adapter segments, in particular configured to accommodate a single reporter/selected from no more than two reporter categories;
annealing a second reporter to at least one of the plurality of second adapter segments such that an oligo tethered to the second reporter is reverse complementary to the second adapter second common region; and detecting the second reporter, in particular without annealing a third reporter to the at least one of the plurality of first adapter segments.

### METHODS AND EXAMPLES

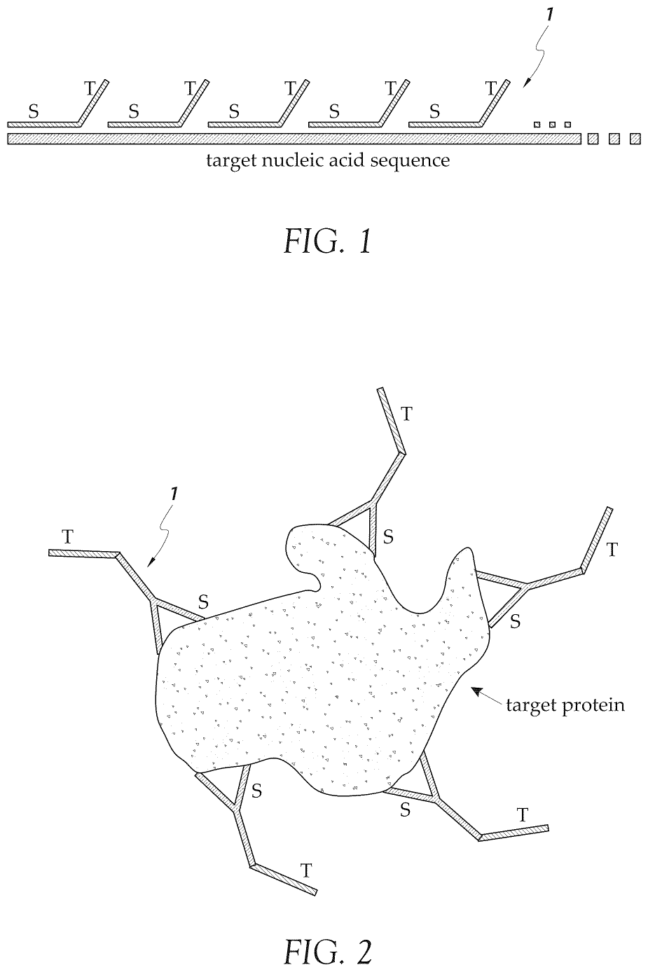

In an application variant, the analyte or target is nucleic acid, e.g., DNA or RNA, and the probe set comprises oligonucleotides that are partially or completely complementary to the whole sequence or a subsequence of the nucleic acid sequence to be detected (FIG. 1). The nucleic acid sequence specific oligonucleotide probe sets comprising analyte-specific probes (1) including a binding element(S) that specifically hybridizes to the target nucleic acid sequence to be detected, and an identifier element (T) comprising a nucleotide sequence which is unique to said set of analyte-specific probes (unique identifier sequence).

In a further application variant, the analyte or target is a protein and the probe set comprises one or more proteins, e.g. antibodies (FIG. 2). The protein specific probe set comprising analyte-specific probes (1) including a binding element (T) such as the (hyper-) variable region of an antibody, that specifically interacts with the target protein to be detected, and the identifier element (T).

In a further application variant, at least one analyte is a nucleic acid and at least a second analyte is a protein and at least the first probe set binds to the nucleic acid sequence and at least the second probe set binds specifically to the protein analyte. Other combinations are possible as well.

An embodiment of the general method of the present disclosure may be:
Step 1: Applying the at least 20 analyte- or target-specific probe sets. The target nucleic acid sequence is incubated with a probe set consisting of oligonucleotides with sequences complementary to the target nucleic acid. In this example, a probe set of 5 different probes is shown, each comprising a sequence element complementary to an individual subsequence of the target nucleic acid sequence (S1 to S5). In this example, the regions do not overlap. Each of the oligonucleotides targeting the same nucleic acid sequence comprises the identifier element or unique identifier sequence (T), respectively.
Step 2: Hybridization of the probe set. The probe set is hybridized to the target nucleic acid sequence under conditions allowing a specific hybridization. After the incubation, the probes are hybridized to their corresponding target sequences and provide the identifier element (T) for the next steps.
Step 3: Eliminating non-bound probes. After hybridization, the unbound oligonucleotides are eliminated, e.g. by washing steps.
Step 4: Applying the decoding oligonucleotides. The decoding oligonucleotides consisting of at least one, preferably at least two sequence elements (t) and (c) are applied. While sequence element (t) is complementary to the unique identifier sequence (T), the sequence element (c) provides a region for the subsequent hybridization of signal oligonucleotides (translator element).
Step 5: Hybridization of decoding oligonucleotides. The decoding oligonucleotides are hybridized with the unique identifier sequences of the probes (T) via their complementary first sequence elements (t). After incubation, the decoding oligonucleotides provide the translator sequence element (c) for a subsequent hybridization step.
Step 6: Eliminating the excess of decoding oligonucleotides. After hybridization, the unbound decoding oligonucleotides are eliminated, e.g. by washing steps.
Step 7: Applying the signal oligonucleotide. The signal oligonucleotides are applied. The signal oligonucleotides comprise at least one second connector element (C) that is essentially complementary to the translator sequence element (c) and at least one signal element that provides a detectable signal (F).
Step 8: Hybridization of the signal oligonucleotides. The signal oligonucleotides are hybridized via the complementary sequence connector element (C) to the translator element (c) of decoding oligonucleotide. After incubation, the signal oligonucleotides are hybridized to their corresponding decoding oligonucleotides and provide a signal (F) that can be detected.
Step 9: Eliminating the excess of signal oligonucleotides. After hybridization, the unbound signal oligonucleotides are eliminated, e.g. by washing steps.
Step 10: Signal detection. The signals provided by the signal oligonucleotides are detected.
The following steps (steps 11 and 12) are unnecessary for the last detection round.
Step 11: Selective denaturation. The hybridization between the unique identifier sequence (T) and the first sequence element (t) of the decoding oligonucleotides is dissolved. The destabilization can be achieved via different mechanisms well known to the trained person like for example: increased temperature, denaturing agents, etc. The target- or analyte-specific probes are not affected by this step.
Step 12: Eliminating the denatured decoding oligonucleotides. The denatured decoding oligonucleotides and signal oligonucleotides are eliminated (e.g. by washing steps) leaving the specific probe sets with free unique identifier sequences, reusable in a next round of hybridization and detection (steps 4 to 10). This detection cycle (steps 4 to 12) is repeated at least four times until the planed encoding scheme is completed.

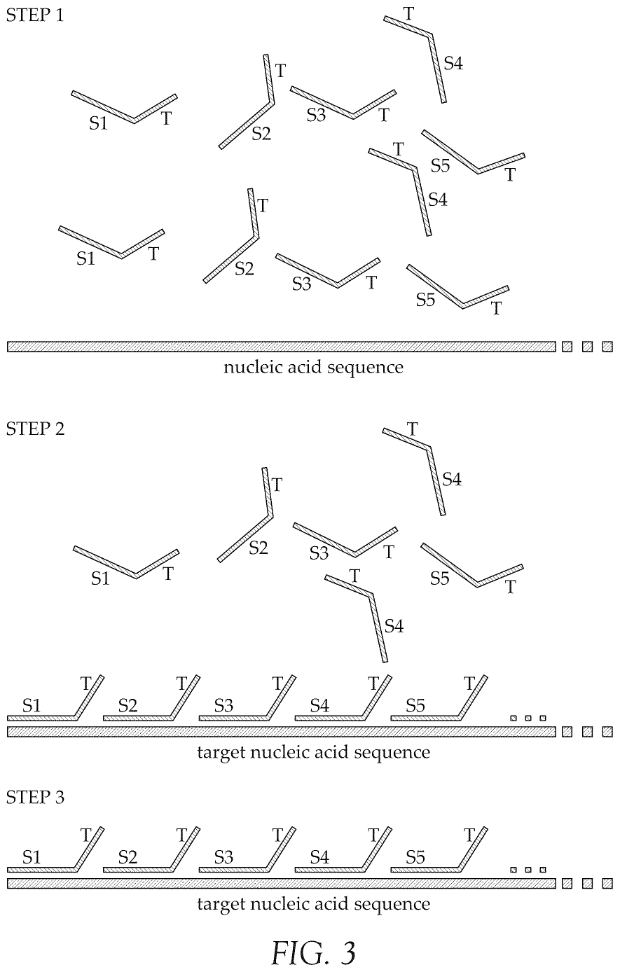

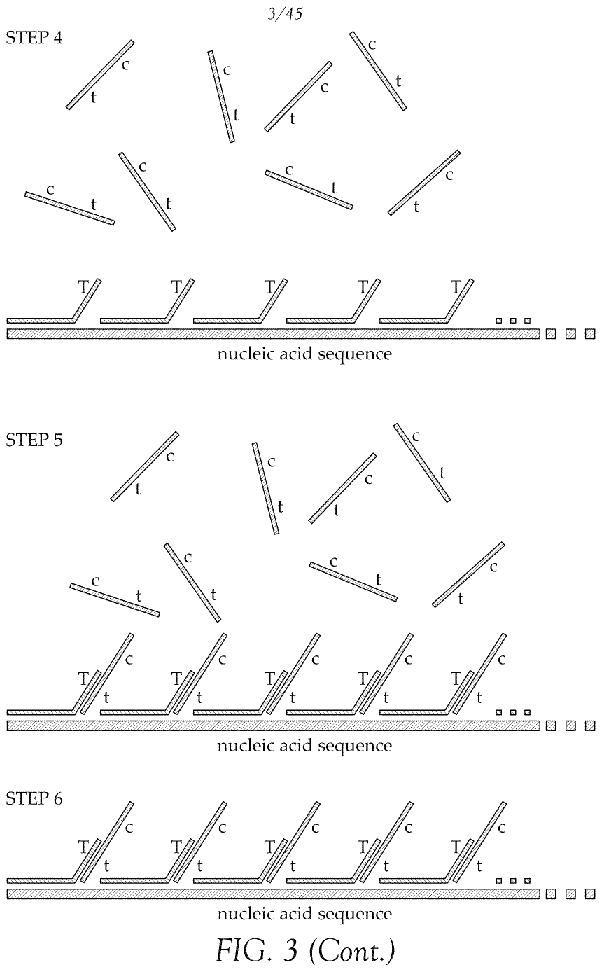

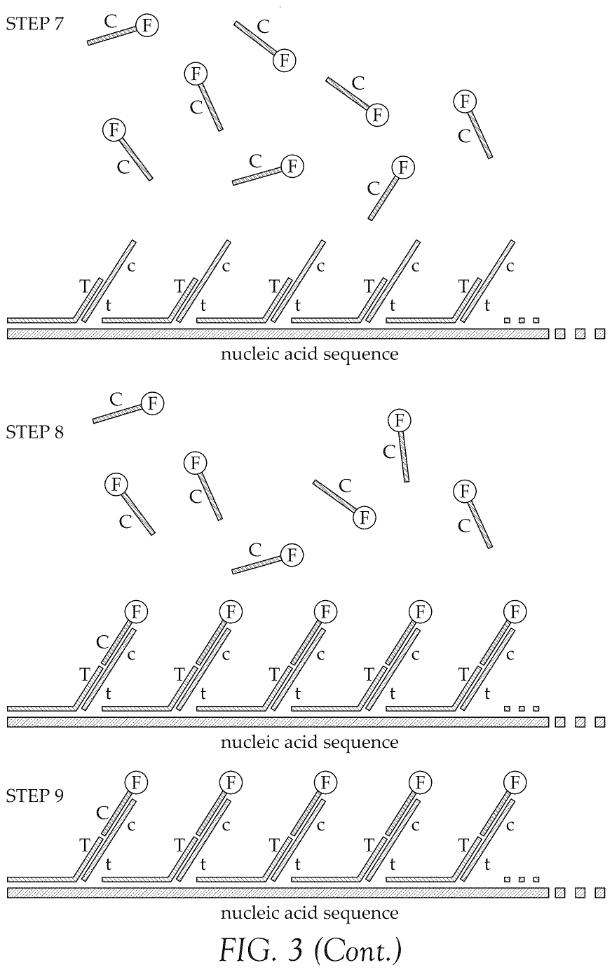

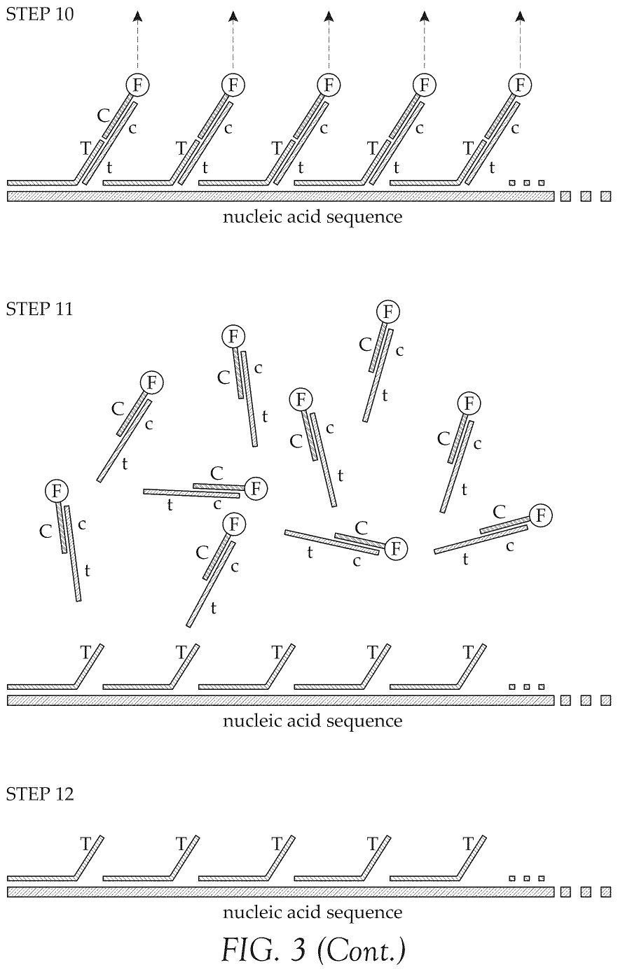

Another embodiment of the general method of the present disclosure using multi-decoders may be (FIG. 16):
Step 1: Target nucleic acids: In this example three different target nucleic acids (A), (B) and (C) have to be detected and differentiated by using only two different types of signal oligonucleotides. Before starting the experiment, a certain encoding scheme is set. In this example, the three different nucleic acid sequences are encoded by three rounds of detection with three different signal types (1), (2) and (1/2) and a resulting hamming distance of 3 to allow for error detection. The planed code words are:
sequence A: (1)-(1)-(2)
sequence B: (2)-(2)-(1/2)
sequence C: (1/2)-(1/2)-(1)
Step 2: Hybridization of the probe sets: For each target nucleic acid, an own probe set is applied, specifically hybridizing to the corresponding nucleic acid sequence of interest. Each probe set provides a unique identifier sequence (T1), (T2) or (T3). This way each different target nucleic acid is uniquely labeled. In this example sequence (A) is labeled with (T1), sequence (B) with (T2) and sequence (C) with (T3). The illustration in FIG. 16 summarizes Steps 1 to 3 of FIG. 3.
Step 3: Hybridization of the decoding oligonucleotides and multi-decoders: For each unique identifier present, a certain decoding oligonucleotide or multi-decoder is applied specifically hybridizing to the corresponding unique identifier sequence by its first sequence element (here (t1) to (T1), (t2) to (T2) and (t3) to (T3)). Each of the decoding oligonucleotides or multi-decoders provides a translator or two translator elements that define the signals that will be generated after hybridization of signal oligonucleotides. Here nucleic acid sequence (A) is labeled with (c1), (B) is labeled with (c2) and (C) is labeled with both translator elements (c1) and (c2) resulting in the signal (1/2). The illustration in FIG. 16 summarizes steps 4 to 6 of FIG. 3.
Step 4: Hybridization of signal oligonucleotides: For each type of translator element, a signal oligonucleotide with a certain signal, differentiable from signals of other signal oligonucleotides, is applied. This signal oligonucleotide can specifically hybridize to the corresponding translator element. The illustration in FIG. 16 summarizes steps 7 to 9 of FIG. 3
Step 5: Signal detection for the encoding scheme: The different signals are detected. Note that in this example the nucleic acids (A), (B) and (C) can already be distinguished after the first round of detection. This is in contrast to the step 5 of FIG. 5 explained by the additional signal type (1/2) that can be realized due to multi-decoders. Although nucleic acid sequences can already be distinguished, the additional rounds contribute to the planned hamming distance of 3. The illustration in FIG. 16 corresponds to step 10 of FIG. 3.
Step 6: Selective denaturation: The decoding (and signal) oligonucleotides and/or multi-decoders of all nucleic acid sequences to be detected are selectively denatured and eliminated as described in steps 11 and 12 of FIG. 3. Afterwards the unique identifier sequences of the different probe sets can be used for the next round of hybridization and detection.
Step 7: Second round of detection: A next round of hybridization and detection is done as described in steps 3 to 5. Note that in this new round the mix of different decoding oligonucleotides and multi-decoders is changed. For example, decoding oligonucleotide of nucleic acid sequence (A) used in the first round comprised of sequence elements (t1) and (c1) while the new multi decoder of round 2 comprises of the sequence elements (t1), (c1) and (c2). Note that now a hamming distance of 2 is already given after 2 rounds, which is the final result of the example in FIG. 3 after 3 rounds.
Step 8: Third round of detection: Again, a new combination of decoding oligonucleotides and/or multi-decoders is used leading to new signal combinations. After signal detection, the resulting code words for the three different nucleic acid sequences are not only unique and therefore distinguishable but comprise a hamming distance of 3 to other code words. Due to the hamming distance, an error in the detection of the signals (signal exchange) would not result in a valid code word and therefore could be detected and because of hamming distance 3 also corrected, in contrast to the encoding scheme of FIG. 3. This way three different nucleic acids can be distinguished in three detection rounds with two different signals, allowing an error detection and correction.

Note that in every round of detection, the type of signal provided by a certain unique identifier is controlled by the use of a certain decoding oligonucleotide. As a result, the sequence of decoding oligonucleotides applied in the detection cycles transcribes the binding specificity of the probe set into a unique signal sequence.

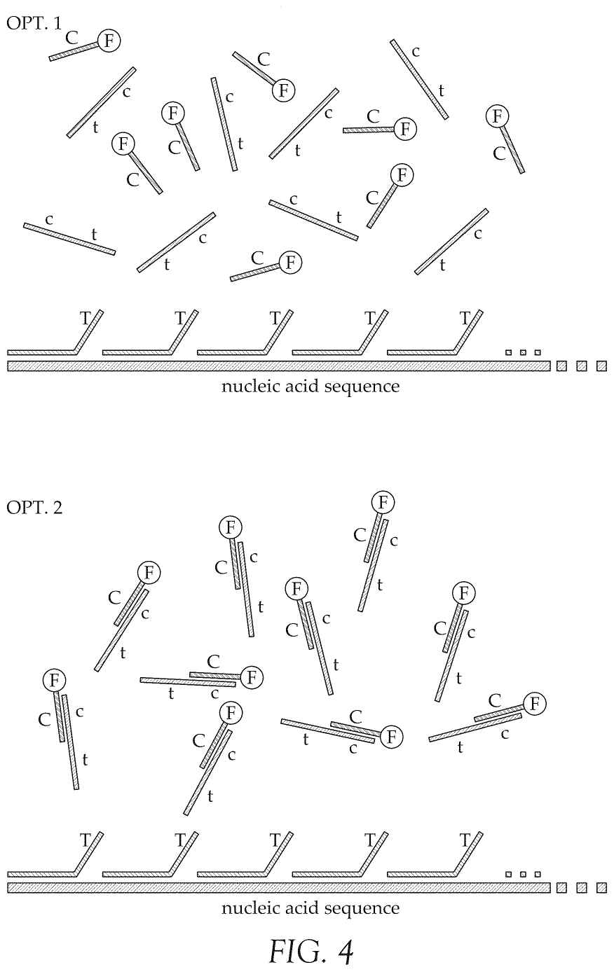

The steps of decoding oligonucleotide hybridization (steps 4 to 6) and signal oligonucleotide hybridization (steps 7 to 9) can also be combined in two alternative ways as shown in FIG. 4.

Opt. 1: Simultaneous hybridization. Instead of the steps 4 to 9 of FIG. 3, specific hybridization of decoding oligonucleotides and signal oligonucleotides can also be done simultaneously leading to the same result as shown in step 9 of FIG. 3, after eliminating the excess decoding- and signal oligonucleotides.

Opt. 2: Preincubation. Additionally to option 1 of FIG. 3, decoding- and signal oligonucleotides can be preincubated in a separate reaction before being applied to the target nucleic acid with the already bound specific probe set.

### 1. Example for Signal Encoding of Three Different Nucleic Acid Sequences by Two Different Signal Types and Three Detection Rounds

FIG. 3 shows the general concept of generation and detection of specific signals mediated by decoding oligonucleotides. It does not show the general concept of encoding that can be achieved by this procedure. To illustrate the use of the process shown in FIG. 3 for the generation of an encoding scheme, FIG. 5 shows a general example for a multiple round encoding experiment with three different nucleic acid sequences. In this example, the encoding scheme includes error detection.

Step 1: Target nucleic acids. In this example three different target nucleic acids (A), (B) and (C) have to be detected and differentiated by using only two different types of signal. Before starting the experiment, a certain encoding scheme is set. In this example, the three different nucleic acid sequences are encoded by three rounds of detection with two different signals (1) and (2) and a resulting hamming distance of 2 to allow for error detection. The planed code words are:
sequence A: (1)-(2)-(2);
sequence B: (1)-(1)-(1);
sequence C: (2)-(1)-(2).

Step 2: Hybridization of the probe sets. For each target nucleic acid, an own probe set is applied, specifically hybridizing to the corresponding nucleic acid sequence of interest. Each probe set provides a unique identifier sequence (T1), (T2) or (T3). This way each different target nucleic acid is uniquely labeled. In this example sequence (T) is labeled with (T1), sequence (B) with (T2) and sequence (C) with (T3). The illustration summarizes steps 1 to 3 of FIG. 3.

Step 3: Hybridization of the decoding oligonucleotides. For each unique identifier present, a certain decoding oligonucleotide is applied specifically hybridizing to the corresponding unique identifier sequence by its first sequence element (here (t1) to (T1), (t2) to (T2) and (t3) to (T3)). Each of the decoding oligonucleotides provides a translator element that defines the signal that will be generated after hybridization of signal oligonucleotides. Here nucleic acid sequences (A) and (B) are labeled with the translator element (c1) and sequence (C) is labeled with (c2). The illustration summarizes steps 4 to 6 of FIG. 3.

Step 4: Hybridization of signal oligonucleotides. For each type of translator element, a signal oligonucleotide with a certain signal (2), differentiable from signals of other signal oligonucleotides, is applied. This signal oligonucleotide can specifically hybridize to the corresponding translator element. The illustration summarizes steps 7 to 9 of FIG. 3.

Step 5: Signal detection for the encoding scheme. The different signals are detected. Note that in this example the nucleic acid sequence (C) can be distinguished from the other sequences by the unique signal (2) it provides, while sequences (A) and (B) provide the same kind of signal (1) and cannot be distinguished after the first cycle of detection. This is due to the fact, that the number of different nucleic acid sequences to be detected exceeds the number of different signals available. The illustration corresponds to step 10 of FIG. 3.

Step 6: Selective denaturation. The decoding (and signal) oligonucleotides of all nucleic acid sequences to be detected are selectively denatured and eliminated as described in steps 11 and 12 of FIG. 3. Afterwards the unique identifier sequences of the different probe sets can be used for the next round of hybridization and detection.

Step 7: Second round of detection. A next round of hybridization and detection is done as described in steps 3 to 5. Note that in this new round the mix of different decoding oligonucleotides is changed. For example, decoding oligonucleotide of nucleic acid sequence (A) used in the first round comprised of sequence elements (t1) and (c1) while the new decoding oligonucleotide comprises of the sequence elements (t1) and (c2). Note that now all three sequences can clearly be distinguished due to the unique combination of first and second round signals.

Step 8: Third round of detection. Again, a new combination of decoding oligonucleotides is used leading to new signal combinations. After signal detection, the resulting code words for the three different nucleic acid sequences are not only unique and therefore distinguishable but comprise a hamming distance of 2 to other code words. Due to the hamming distance, an error in the detection of the signals (signal exchange) would not result in a valid code word and therefore could be detected. By this way three different nucleic acids can be distinguished in three detection rounds with two different signals, allowing error detection.

### 2. Advantages Over Prior Art Technologies

### Coding Strategy

Compared to state-of-the-art methods, one particular advantage of the method according to the disclosure is the use of decoding oligonucleotides breaking the dependencies between the target specific probes and the signal oligonucleotides.

Without decoupling target specific probes and signal generation, two different signals can only be generated for a certain target if using two different molecular tags. Each of these molecular tags can only be used once. Multiple readouts of the same molecular tag do not increase the information about the target. In order to create an encoding scheme, a change of the target specific probe set after each round is required (SeqFISH) or multiple molecular tags must be present on the same probe set (like merFISH, intronSeqFISH).

Following the method according to the disclosure, different signals are achieved by using different decoding oligonucleotides reusing the same unique identifier (molecular tag) and a small number of different, mostly cost-intensive signal oligonucleotides. This leads to several advantages in contrast to the other methods.
(1) The encoding scheme is not defined by the target specific probe set as it is the case for all other methods of prior art. Here the encoding scheme is transcribed by the decoding oligonucleotides. This leads to a much higher flexibility concerning the number of rounds and the freedom in signal choice for the codewords. Looking on the methods of prior art (e.g. merFISH or intronSeqFISH), the encoding scheme (number, type and sequence of detectable signals) for all target sequences is predefined by the presence of the different tag sequences on the specific probe sets (4 of 16 different tags per probe set in the case of merFISH and 5 of 60 different tags in the case of intron FISH), In order to produce a sufficient number of different tags per probe set, the methods use rather complex oligonucleotide designs with several tags present on one target specific oligonucleotide. In order to change the encoding scheme for a certain target nucleic acid, the specific probe set has to be replaced. The method according to the disclosure describes the use of a single unique tag sequence (unique identifier) per analyte, because it can be reused in every detection round to produce a new information. The encoding scheme is defined by the order of decoding oligonucleotides that are used in the detection rounds. Therefore, the encoding scheme is not predefined by the specific probes (or the unique tag sequence) but can be adjusted to different needs, even during the experiment. This is achieved by simply changing the decoding oligonucleotides used in the detection rounds or adding additional detection rounds.
(2) The number of different signal oligonucleotides must match the number of different tag sequences with methods of prior art (16 in the case of merFISH and 60 in the case of intronSeqFISH). Using the method according to the disclosure, the number of different signal oligonucleotides matches the number of different signals used. Due to this, the number of signal oligonucleotides stays constant for the method described here and never exceeds the number of different signals but increases with the complexity of the encoding scheme in the methods of prior art (more detection rounds more different signal oligonucleotides needed). As a result, the method described here leads to a much lower complexity (unintended interactions of signal oligonucleotides with environment or with each other) and dramatically reduces the cost of the assay since the major cost factor are the signal oligonucleotides.
   (3) In the methods of prior art, the number of different signals generated by a target specific probe set is restricted by the number of different tag sequences the probe set can provide. Since each additional tag sequence increases the total size of the target specific probe, there is a limitation to the number of different tags a single probe can provide. This limitation is given by the size dependent increase of several problems (unintended inter- and intramolecular interactions, costs, diffusion rate, stability, errors during synthesis etc.). Additionally, there is a limitation of the total number of target specific probes that can be applied to a certain analyte. In case of nucleic acids, this limitation is given by the length of the target sequence and the proportion of suitable binding sites. These factors lead to severe limitations in the number of different signals a probe set can provide (4 signals in the case of merFISH and 5 signals in the case of intronSeqFISH). This limitation substantially affects the number of different code words that can be produced with a certain number of detection rounds. In the approach of the disclosure only one tag is needed and can be freely reused in every detection round. This leads to a low oligonucleotide complexity/length and at the same time to the maximum encoding efficiency possible (number of colors^{number of rounds}). The vast differences of coding capacity of our method compared to the other methods is shown in FIGS. 1 and 5. Due to this in approach of the disclosure a much lower number of detection rounds is needed to produce the same amount of information. A lower number of detection rounds is connected to lower cost, lower experimental time, lower complexity, higher stability and success rate, lower amount of data to be collected and analyzed and a higher accuracy of the results.

### Coding Capacity

All three methods compared in the Table 1 below use specific probe sets that are not denatured between different rounds of detection. For intronSeqFISH there are four detection rounds needed to produce the pseudo colors of one coding round, therefore data is only given for rounds 4, 8, 12, 16 and 20. The merFISH-method uses a constant number of 4 signals, therefore the data starts with the smallest number of rounds possible. After 8 detection rounds our method exceeds the maximum coding capacity reached with 20 rounds of merFISH (depicted with one asterisk) and after 12 rounds of detection the maximum coding capacity of intron FISH is exceeded (depicted with two asterisks). For the method according to the disclosure usage of 3 different signals is assumed (as is with intronSeqFISH).

TABLE 1
Comparison of coding capacity

| | Coding capacity | | |
|---|---|---|---|
| | Method of | | |
| Number of | the present | intron | |
| Rounds: | disclosure | FISH | merFISH |
| 1 | 3 | — | — |
| 2 | 9 | — | — |
| 3 | 27 | — | — |
| 4 | 81 | 12 | 1 |
| 5 | 243 | — | 5 |
| 6 | 729 | — | 15 |
| 7 | 2187 | — | 35 |
| 8* | 6561 | 144 | 70 |
| 9 | 19683 | — | 126 |
| 10 | 59049 | — | 210 |
| 11 | 177147 | — | 330 |
| 12** | 531441 | 1728 | 495 |
| 13 | 1594323 | — | 715 |
| 14 | 4782969 | — | 1001 |
| 15 | 14348907 | — | 1365 |
| 16 | 43046721 | 20736 | 1820 |
| 17 | 129140163 | — | 2380 |
| 18 | 387420489 | — | 3060 |
| 19 | 1162261467 | — | 3876 |
| 20 | 3486784401 | 248832 | 4845 |

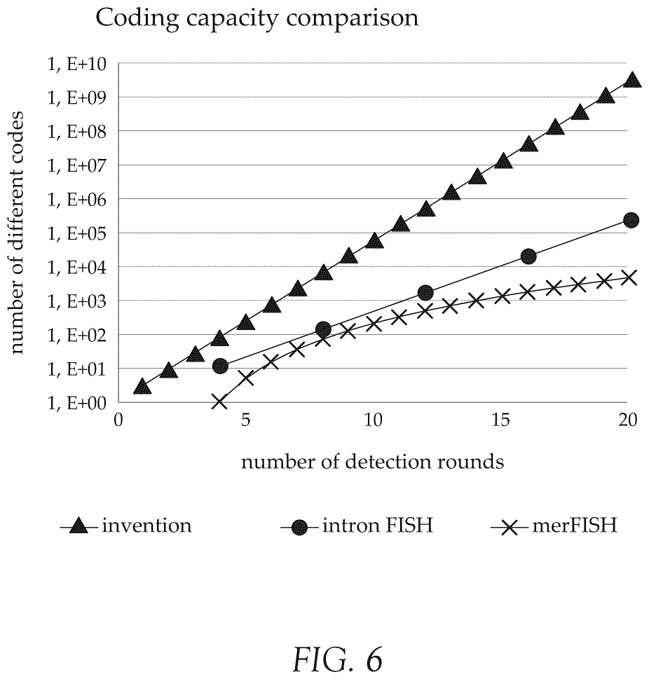

As shown in FIG. 6 the number of codewords for merFISH does not exponentially increase with the number of detection cycles but gets less effective with each added round. In contrast, the number of codewords for intronSeqFISH in the method according to the disclosure increases exponentially. The slope of the curve for the proposed method is much higher than that of intron FISH, leading to more than 10,000 times more code words usable after 20 rounds of detection.

Note that this maximum efficiency of coding capacity is also reached in case of seqFISH, where specific probes are denatured after every detection round and a new probe set is specifically hybridized to the target sequence for each detection round. However, this method has major downsides to technologies using only one specific hybridization for their encoding scheme (all other methods):
(1) For the efficient denaturation of the specific probes, rather crude conditions must be used (high temperatures, high concentrations of denaturing agent, long incubation times) leading to much higher probability for the loss or the damage of the analyte.
(2) For each round of detection an own probe set has to be used for every target nucleic acid sequence. Therefore, the number of specific probes needed for the experiment scales with the number of different signals needed for the encoding scheme. This dramatically increases the complexity and the cost of the assay.
(3) Because the hybridization efficiency of every target nucleic acid molecule is subject to some probabilistic effects, the fluctuations of signal intensity between the different detection rounds is much higher than in methods using only one specific hybridization event, reducing the proportion of complete codes.
(4) The time needed for the specific hybridization is much longer than for the hybridization of signal oligonucleotides or decoding oligonucleotides (as can be seen in the method parts of the intronSeqFISH, merFISH and seqFISH publications), which dramatically increases the time needed to complete an experiment.

Due to these reasons all other methods use a single specific hybridization event and accept the major downside of lower code complexity and therefore the need of more detection rounds and a higher oligonucleotide design complexity.

The method according to the disclosure combines the advantages of seqFISH (mainly complete freedom concerning the encoding scheme) with all advantages of methods using only one specific hybridization event while eliminating the major problems of such methods.

Note that the high numbers of code words produced after 20 rounds can also be used to introduce higher hamming distances (differences) between different codewords, allowing error detection of 1,2 or even more errors and even error corrections. Therefore, even very high coding capacities are still practically relevant.

As mentioned above, the usage of multi-decoders further increases the coding capacity of the encoding scheme. Instead of being limited to the having exactly the same number of different signal types as different signal oligonucleotides and corresponding translator elements, the use of multi-decoders increases the signal types that can be used to: (N×(N+1))/2 (with N being the number of different signal oligonucleotides used). For the code used in table 1 with 3 different signal oligonucleotides this would mean the following 7 different signal types could be used: (S1), (S2), (S3), (S1/S2), (S1/S3), (S2/S3), (S1/S2/S3). The effect to the coding efficiency can be seen in Table 1b and FIG. 17.

| | Coding capacity | | | |
|---|---|---|---|---|
| | | Method of the | | |
| | Method of the | present disclosure | | |
| Number of | present | with multi- | intron | |
| Rounds: | disclosure | decoders | FISH | merFISH |
| 1 | 3 | 7 | — | — |
| 2 | 9 | 49 | — | — |
| 3 | 27 | 343 | — | 2 |
| 4 | 81 | 2401 | 12 | 1 |
| 5* | 243 | 16807 | — | 5 |
| 6 | 729 | 117649 | — | 15 |
| 7** | 2187 | 823543 | — | 35 |
| 8 | 6561 | 5764801 | 144 | 70 |
| 9 | 19683 | 40353607 | — | 126 |
| 10 | 59049 | 282475249 | — | 210 |
| 11 | 177147 | 1977326743 | — | 330 |
| 12*** | 531441 | 1.3841 × 10\hat{ }10 | 1728 | 495 |
| 13 | 1594323 | 9.6889 × 10\hat{ }10 | — | 715 |
| 14 | 4782969 | 6.7822 × 10\hat{ }11 | — | 1001 |
| 15 | 14348907 | 4.7476 × 10\hat{ }12 | — | 1365 |
| 16 | 43046721 | 3.3233 × 10\hat{ }13 | 20736 | 1820 |
| 17 | 129140163 | 2.3263 × 10\hat{ }14 | — | 2380 |
| 18 | 387420489 | 1.6284 × 10\hat{ }15 | — | 3060 |
| 19 | 1162261467 | 1.1399 × 10\hat{ }16 | — | 3876 |
| 20 | 3486784401 | 7.9792 × 10\hat{ }16 | 248832 | 4845 |

Table 1b shows the coding capacity of the four methods. All four methods compared in the table use specific probe sets that are not denatured between different rounds of detection. For intronSeqFISH there are four detection rounds needed to produce the pseudo colors of one coding round, therefore data is only given for rounds 4, 8, 12, 16 and 20. The merFISH-method uses a constant number of 4 signals, therefore the data starts with the smallest number of rounds possible. After 4 detection rounds the method with multi-decoders as described here exceeds the maximum coding capacity reached with 20 rounds of merFISH (depicted with one asterisk), after 7 rounds of detection the maximum coding capacity of intron FISH is exceeded (depicted with two asterisks) and after 12 rounds of detection the maximum coding capacity of the method of the present disclosure is exceeded (depicted with three asterisks). The usage of 3 different signal oligonucleotides is assumed (as is with intronSeqFISH).

### 3. Selective Denaturation, Oligonucleotide Assembly and Reuse of Unique Identifiers are Surprisingly Efficient

A key factor of the method according to the disclosure is the consecutive process of decoding oligonucleotide binding, signal oligonucleotide binding, signal detection and selective denaturation. In order to generate an encoding scheme, this process has to be repeated several times (depending on the length of the code word). Because the same unique identifier is reused in every detection cycle, all events from the first to the last detection cycle are depending on each other. Additionally, the selective denaturation depends on two different events: While the decoding oligonucleotide has to be dissolved from the unique identifier with highest efficiency, specific probes have to stay hybridized with highest efficiency.

Due to this the efficiency E of the whole encoding process can be described by the following equation:

E=Bₛₚ×(B_{de}×Bₛᵢ×E_{de}×Sₛₚ)ⁿ
E=total efficiency
Bₛₚ=binding of specific probes
B_{de}=binding of decoding oligonucleotides
Bₛᵢ=binding of signal oligonucleotides
E_{de}=elimination of decoding oligonucleotides
Sₛₚ=stability of specific probes during elimination process
n=number of detection cycles

Based on this equation the efficiency of each single step can be estimated for a given total efficiency of the method. The calculation is hereby based on the assumption, that each process has the same efficiency. The total efficiency describes the portion of successfully decodable signals of the total signals present.

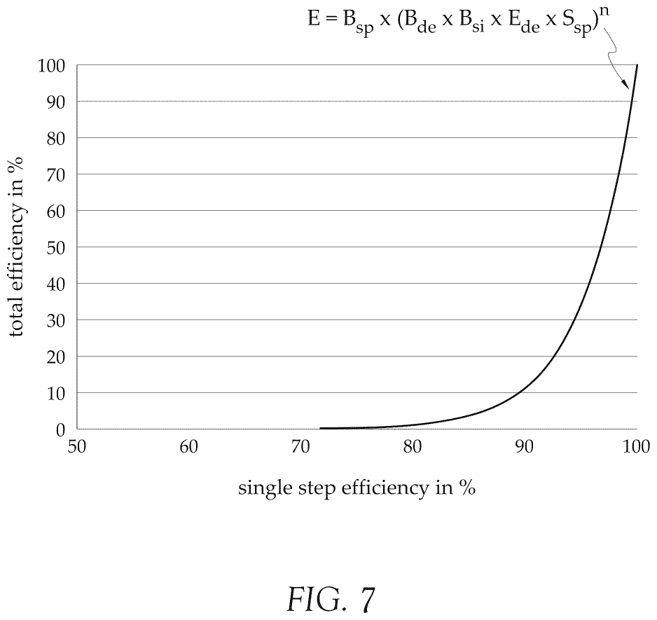

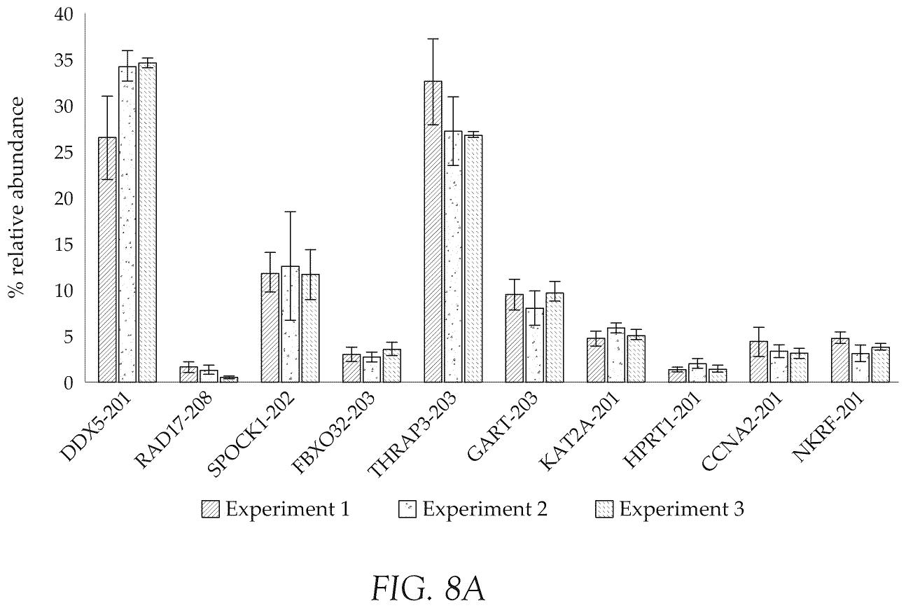

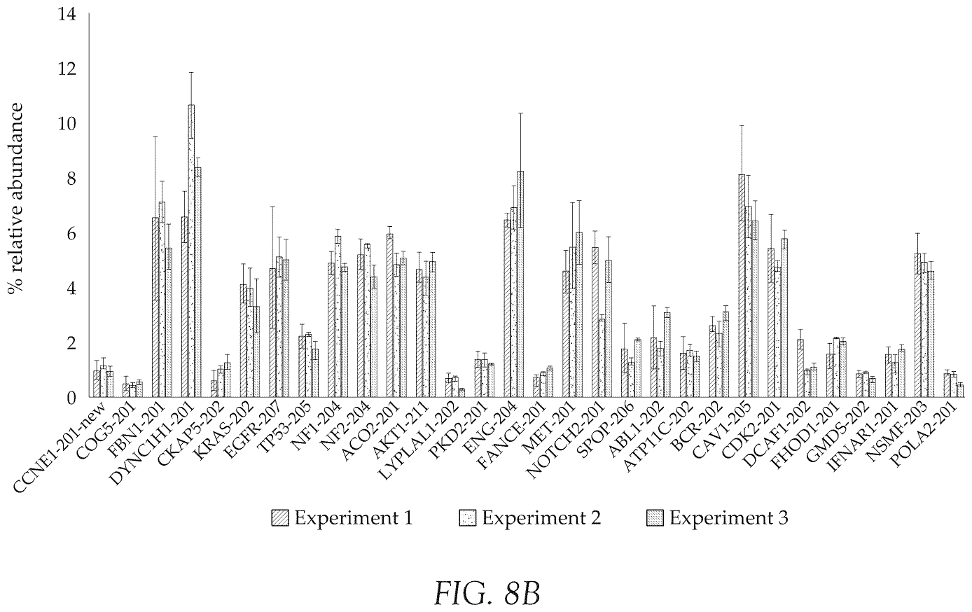

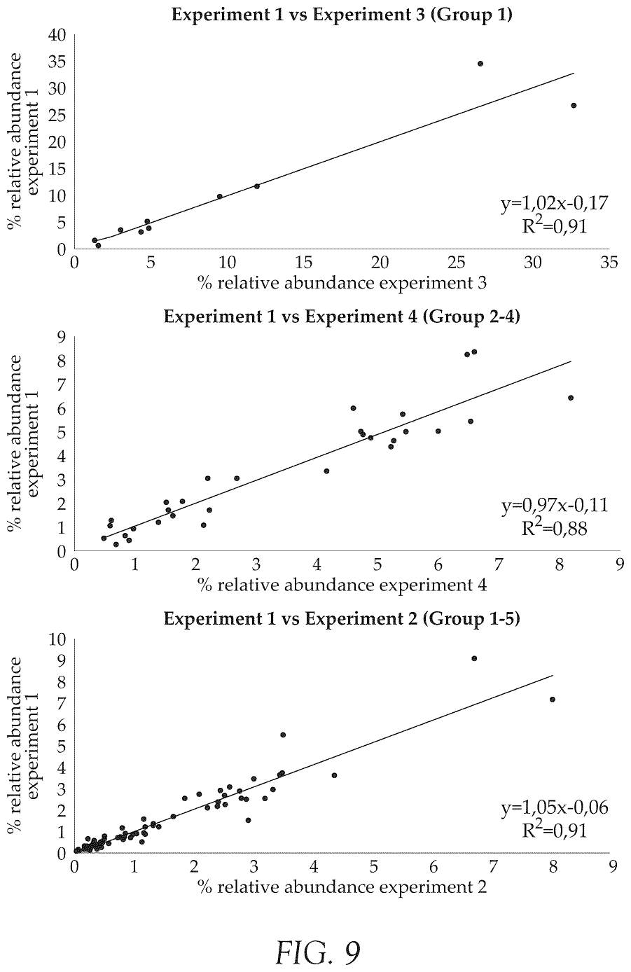

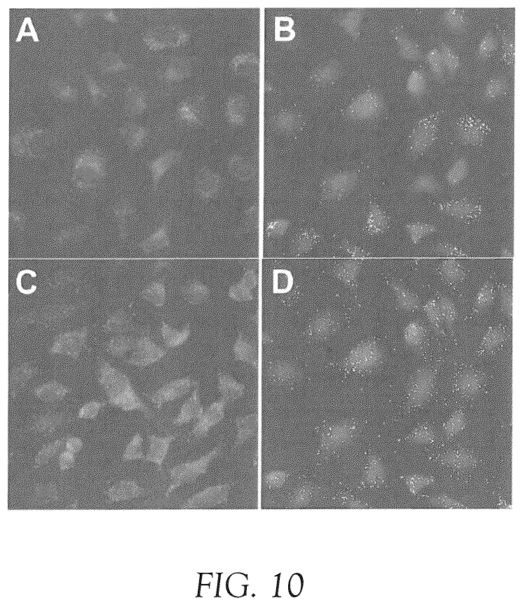

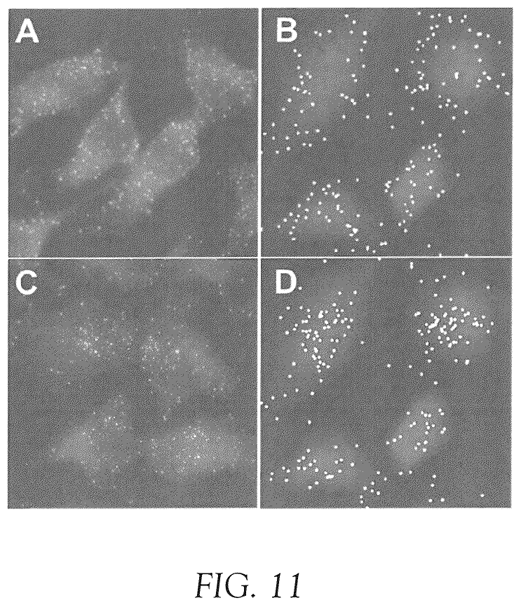

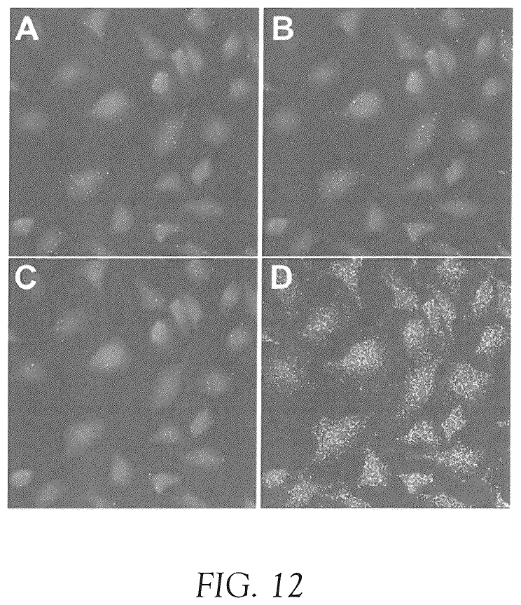

The total efficiency of the method is dependent on the efficiency of each single step of the different factors described by the equation. Under the assumption of an equally distributed efficiency the total efficiency can be plotted against the single step efficiency as shown in FIG. 7. As can be seen, a practically relevant total efficiency for an encoding scheme with 5 detection cycles can only be achieved with single step efficiencies clearly above 90%. For example, to achieve a total efficiency of 50% an average efficiency within each single step of 97.8% is needed. These calculations are even based on the assumption of a 100% signal detection and analysis efficiency. Due to broad DNA melting curves of oligonucleotides of a variety of sequences, the inventors assumed prior to experiments that the selective denaturation would work less efficient for denaturation of decoding oligonucleotides and that sequence specific binding probes are not stable enough. In contrast to this assumption, we found a surprising effectiveness of all steps and a high stability of sequence specific probes during selective denaturation.

Experimentally, the inventors achieved a total decoding efficiency of about 30% to 65% based on 5 detection cycles. A calculation of the efficiency of each single step (Bsp, Bde, Bsi, Ede, Ssp) by the formula given above revealed an average efficiency of about 94.4% to 98%. These high efficiencies are very surprising and cannot easily be anticipated by a well-trained person in this field.

### 4. Experimental Data

1. Example for signal encoding of two subgroups of nucleic acid sequences with spatial overlap The table below shows examples of implementations using two subgroups (analytic sets) in consecutive runs:

| | | Tran- | | | | |
|---|---|---|---|---|---|---|
| | | script | | | | |
| | | Plexity | Run 1 | | Run 2 | |
| | Total | (Set | Fluidics | Plexity | Fluidics | Plexity |
| | rounds | 1/Set 2) | rounds | 1 | rounds | 2 |
| 1.0 | 9 | 100/2 | 8 | 100 | 1 | 2 |
| | | | | (129) | | |
| 2.0 | 16 | 300/25 | 10 | 300 | 6 | 25 |
| | | | | (419) | | (29) |
| 2.1 | 16 | 500/50 | 10 | 500 | 6 | 50 |
| Improved | | | | (685) | | (60) |
| transcript | | | | | | |
| detection | | | | | | |

For “run 1.0” n=8 rounds of hybridization and imaging was used to detect 100 genes. For “run 2.0” the number of rounds is increased to n=10 and can thereby target 300 genes. For “run 2.1” an enhanced transcript detection probe is used which allows to target 500 transcripts with the same number of rounds (n=10).

Consecutive runs are run on the same tissue sections to detect transcripts beyond the diffraction limit.

The main steps of the method are
i. Hybridization of target probes of the two analytic sets happen simultaneously before the first run.
ii. Tails of the target probes are unique for both runs. e.g., 100, 300 and 500 tails (for 100, 300, or 500 transcripts, respectively) are used for the first run with the first analytic set and 2, 25, 50 tails (for 2, 25, 50 transcripts, respectively) for the second run with the second analytic set. This requires also different decoder sets for both runs.
iii. Both runs generate independent datasets that can be combined in-silico afterwards. Thereby, 1) enhancing the multiplexing capability without increasing optical crowding and 2) detecting spatial overlapping transcripts which would be otherwise not possible due to the diffraction limit of the microscope. This is e.g. allows for detection of fusion genes (cancer) or for detection of co-localization of different transcript types (e.g. transcriptional hubs in the nucleus). In addition, transcripts with higher expression levels can also be analyzed because the “signal spread” of the high number of abundant signals vs. the detection of other (especially lowly expressed) genes is improved.

In principle, both runs can be also interwoven, e.g.:
Detection round 1=1st round of detection of first analytical set
Detection round 2=1st round of detection of second analytical set
Detection round 3=2nd round of detection of first analytical set
Detection round 4=2nd round of detection of second analytical set
Etc.

Sequences
Sequence of Tails attached to particle used in this disclosure

| | Cat | | | | | | |
|---|---|---|---|---|---|---|---|
| No | No. | Species | Gene | Target | Tail | Code | Tall Sequence |
| 8 | PDX1J | Hs | EGFR | ENSG000001 | 2 | XYRYXR | tagacAGATGTGGTTAGT |
| | | | | 46648 | | YX | CT (SEQ ID NO: 1) |
| 15 | PQ02S | Hs | BRCA1 | ENSG000000 | 12 | XRYYRX | tagacCTAATCTTACTTA |
| | | | | 12048 | | YX | TAACG (SEQ ID NO: 2) |
| 11 | PJ71C | Hs | APC | ENSG000001 | 13 | YRXRRX | tagacGAAGGATCGGAAA |
| | | | | 34982 | | YX | G (SEQ ID NO: 3) |
| 18 | PY02L | Hs | AOX1 | ENSG000001 | 24 | RRXXYY | tagacAGAATCAAAAGTG |
| | | | | 38356 | | YX | AAC (SEQ ID NO: 4) |
| 14 | PPA1D | Hs | E2F1 | ENSG000001 | 30 | RRYXYX | tagacATCTAGGCACTTT |
| | | | | 01412 | | RX | AC (SEQ ID NO: 5) |
| 19 | PZX13 | Hs | BCR | ENSG000001 | 62 | RYXXRY | tagacAAGCTTCTATTAG |
| | | | | 86716 | | XY | CT (SEQ ID NO: 6) |
| 5 | P6127 | Hs | CDK1 | ENSG000001 | 64 | YXXYRX | tagacGTTTTCGGCATTG |
| | | | | 70312 | | YR | (SEQ ID NO: 7) |
| 9 | PEX1K | Hs | TP53 | ENSG000001 | 69 | RYXXYX | tagacTGCTATAAGAGTC |
| | | | | 41510 | | RY | CC (SEQ ID NO: 8) |
| 10 | PJ31G | Hs | DDX5 | ENSG000001 | 70 | RYXYRX | tagacGCTTACTCGAAGA |
| | | | | 08654 | | XR | C (SEQ ID NO: 9) |
| 1 | P1A1S | Hs | BIRC5 | ENSG000000 | 72 | XYRXYX | tagacGCAAACATCTTTG |
| | | | | 89685 | | RR | G (SEQ ID NO: 10) |
| 3 | P4125 | Hs | CDC25C | ENSG000001 | 82 | XYYRXY | tagacAATACTAGAAAGT |
| | | | | 58402 | | XY | ATCTG (SEQ ID NO: 11) |
| 6 | P8129 | Hs | CENPF | ENSG000001 | 94 | RXXRYX | tagacTACTGATACAAGT |
| | | | | 17724 | | RR | AGAT (SEQ ID NO: 12) |
| 17 | PT71M | Hs | BCL2 | ENSG000001 | 106 | YXYYRX | tagacAACCTAAAGTTGC |
| | | | | 71791 | | XY | TA (SEQ ID NO: 13) |
| 13 | PNT1X | Hs | AKT1 | ENSG000001 | 110 | XRXYYY | tagacCTTAAGTCTTAAT |
| | | | | 42208 | | XY | GAAAG (SEQ ID NO: 14) |
| 7 | PCX1H | Hs | KRAS | ENSG000001 | 113 | RXXRRR | tagacGAAACGTATATTT |
| | | | | 33703 | | XY | TCC (SEQ ID NO: 15) |
| 16 | PQ61J | Hs | AURKB | ENSG000001 | 115 | YXRXXR | tagacCCGTATAGGATAC |
| | | | | 78999 | | RR | AG (SEQ ID NO: 16) |
| 4 | P4X19 | Hs | HPRT1 | ENSG000001 | 117 | XRRRXY | tagacCATATTCGTAACA |
| | | | | 65704 | | XR | CG (SEQ ID NO: 17) |
| 12 | PJX1Q | Hs | ACO2 | ENSG000001 | 118 | YYYXYX | tagacTTGCTACTAGTAA |
| | | | | 00412 | | XR | GTG (SEQ ID NO: 18) |
| 2 | P2123 | Hs | CCNE1 | ENSG000001 | 126 | RRRXXR | tagacCCATAGCTTATTT |
| | | | | 05173 | | XY | AAGC (SEQ ID NO: 19) |

Sequences of Decoders used in this disclosure

| Tail | C1 (Read Out-Yellow) | C2 (Read Out-Red) |
|---|---|---|
| T2 | ACCTTAAGAATGGAATAGACTAA | GGCTAATTTATTACCTAGACTA |
| | CCACATCT (SEQ ID NO: 20) | ACCACATCT (SEQ ID NO: 21) |
| T12 | ACCTTAAGAATGGAATCGTTATA | GGCTAATTTATTACCTCGTTAT |
| | AGTAAGATTAG (SEQ ID NO: 22) | AAGTAAGATTAG (SEQ ID NO: |
| | | 23) |
| T13 | ACCTTAAGAATGGAATCTTTCCG | GGCTAATTTATTACCTCTTTCCG |
| | ATCCTTC (SEQ ID NO: 24) | ATCCTTC (SEQ ID NO: 25) |
| T24 | ACCTTAAGAATGGAATGTTCACT | GGCTAATTTATTACCTGTTCACT |
| | TTTGATTCT (SEQ ID NO: 26) | TTTGATTCT (SEQ ID NO: 27) |
| T30 | ACCTTAAGAATGGAATGTAAAGT | GGCTAATTTATTACCTGTAAAG |
| | GCCTAGAT (SEQ ID NO: 28) | TGCCTAGAT (SEQ ID NO: 29) |
| T62 | ACCTTAAGAATGGAATAGCTAAT | GGCTAATTTATTACCTAGCTAA |
| | AGAAGCTT (SEQ ID NO: 30) | TAGAAGCTT (SEQ ID NO: 31) |
| T64 | ACCTTAAGAATGGAATCAATGCC | GGCTAATTTATTACCTCAATGCC |
| | GAAAAC (SEQ ID NO: 32) | GAAAAC (SEQ ID NO: 33) |
| T69 | ACCTTAAGAATGGAATGGGACTC | GGCTAATTTATTACCTGGGACTC |
| | TTATAGCA (SEQ ID NO: 34) | TTATAGCA (SEQ ID NO: 35) |
| T70 | ACCTTAAGAATGGAATGTCTTCG | GGCTAATTTATTACCTGTCTTCG |
| | AGTAAGC (SEQ ID NO: 36) | AGTAAGC (SEQ ID NO: 37) |
| T72 | ACCTTAAGAATGGAATCCAAAGA | GGCTAATTTATTACCTCCAAAG |
| | TGTTTGC (SEQ ID NO: 38) | ATGTTTGC (SEQ ID NO: 39) |
| T82 | ACCTTAAGAATGGAATCAGATAC | GGCTAATTTATTACCTCAGATA |
| | TTTCTAGTATT (SEQ ID NO: 40) | CTTTCTAGTATT (SEQ ID NO: |
| | | 41) |
| T94 | ACCTTAAGAATGGAATATCTACT | GGCTAATTTATTACCTATCTACT |
| | TGTATCAGTA (SEQ ID NO: 42) | TGTATCAGTA (SEQ ID NO: 43) |
| T106 | ACCTTAAGAATGGAATTAGCAAC | GGCTAATTTATTACCTTAGCAA |
| | TTTAGGTT (SEQ ID NO: 44) | CTTTAGGTT (SEQ ID NO: 45) |
| T110 | ACCTTAAGAATGGAATCTTTCAT | GGCTAATTTATTACCTCTTTCAT |
| | TAAGACTTAAG (SEQ ID NO: 46) | TAAGACTTAAG (SEQ ID NO: 47) |
| T113 | ACCTTAAGAATGGAATGGAAAAT | GGCTAATTTATTACCTGGAAAA |
| | ATACGTTTC (SEQ ID NO: 48) | TATACGTTTC (SEQ ID NO: 49) |
| T115 | ACCTTAAGAATGGAATCTGTATC | GGCTAATTTATTACCTCTGTATC |
| | CTATACGG (SEQ ID NO: 50) | CTATACGG (SEQ ID NO: 51) |
| T117 | ACCTTAAGAATGGAATCGTGTTA | GGCTAATTTATTACCTCGTGTTA |
| | CGAATATG (SEQ ID NO: 52) | CGAATATG (SEQ ID NO: 53) |
| T118 | ACCTTAAGAATGGAATCACTTAC | GGCTAATTTATTACCTCACTTAC |
| | TAGTAGCAA (SEQ ID NO: 54) | TAGTAGCAA (SEQ ID NO: 55) |
| T126 | ACCTTAAGAATGGAATGCTTAAA | GGCTAATTTATTACCTGCTTAA |
| | TAAGCTATGG (SEQ ID NO: 56) | ATAAGCTATGG (SEQ ID NO: 57) |

1. A monitoring probe comprising at least one element having a nucleotide sequence, wherein said element is selected from the group consisting of
a translator (c) element comprising a nucleotide sequence allowing the binding/hybridization of a signal oligonucleotide;
an identifier element (t) comprising a nucleotide sequence allowing the binding/hybridization of a decoding element, wherein in particular the identifier element (t) comprising a nucleotide sequence which is essentially complementary to at least a section of the unique set identifier sequence of an identifier element (T) of a corresponding decoding element;
a target nucleic acid sequence allowing the binding/hybridization of a binding element.

2. The monitoring probe according to claim 1 comprising at least one identifier element (t) comprising a nucleotide sequence which is essentially complementary to at least a section of the unique set identifier sequence of an identifier element (T) of a corresponding decoding element.

3. The monitoring probe according to any one of claims 1 to 2, wherein the nucleotide acid sequence of the monitoring probe is attached to a particle selected from a non-magnetic bead particle, magnetic bead particle, a streptavidin coated particle, a biotin coated particle, and/or any combination thereof.

4. The monitoring probe according to any one of claims 1 to 3, wherein the probe further comprises a binding element complementary to at least a section of a target nucleotide acid sequence.

5. The monitoring probe according to any one of claims 1 to 3, wherein the target nucleotide acid sequence is attached to a particle selected from a non-magnetic bead particle, a magnetic bead particle, a streptavidin coated particle, a biotin coated particle, and/or any combination thereof.

6. The monitoring probe according to any of claim 4 or 5, wherein the nucleic acid sequence contains a spacer element between the portion to be immobilized onto the particle and the nucleic acid binding element and/or identifier element (t).

7. The monitoring probe according to any of claims 1 to 6, wherein the monitoring probe is immobilized on a slide surface through a poly-lysine coating of the slide surface.

8. The monitoring probe according to claim 7, wherein the monitoring probe is immobilized to the poly-lysine coated slide surface using homobifunctional or heterobifunctional cross-linking reagents via a reactive group.

9. The monitoring probe according to claim 8, wherein the monitoring probe or the slide surface comprise a reactive group consisting of an alkyne or azide, and the other contact surface comprises the partner azide or alkyne, such that the immobilization can occur using click chemistry.

10. The monitoring probe according to any of claims 1 to 9, wherein the size of the monitoring probe is 250 nm or less.

11. A method to monitor a in situ hybridization multiplex reaction wherein the method comprises at least the steps of:
1. contacting the analyte nucleotide sequence with at least one detecting nucleic acid sequence in order to conduct an in-situ hybridization; and
2. adding the monitoring probe according to any of claims 1 to 10 before, during and/or after adding the detecting nucleic acid sequence in step 1.

12. The method according to claim 11,
wherein the in-situ hybridization multiplex reaction is characterized by comprising adding a:
a.) a binding element which is essentially complementary to at least a section of an analyte nucleotide sequence to be detected and an identifier element (T) comprising a nucleotide sequence which is unique to the analyte nucleotide sequence; and/or
b.) a decoding element comprising an identifier element (t) comprising a nucleotide sequence which is essentially complementary to at least a section of the unique set identifier sequence of the identifier element (T) of the corresponding binding element, and a translator element (c) comprising a nucleotide sequence allowing a specific hybridization of a signal oligonucleotide; and/or
c.) a signal oligonucleotide comprising a translator connector element (C) comprising a nucleotide sequence which is essentially complementary to at least a section of the nucleotide sequence of a translator element (c) comprised in a decoding oligonucleotide, and a signal element facilitating a signal which is specific for the polymorphic analyte; and
wherein the method comprises the monitoring probe according to any of the claims 1 to 10; and
wherein the method comprises at least the steps of:
1. contacting the binding element with the analyte nucleotide sequence;
2. contacting the binding element with the decoding element; and
3. contacting the signal oligonucleotide with the decoding element; and
wherein the monitoring probe is added before, during and/or after adding the decoding element in step 2.

13. The method according to any of claim 11 or 12, wherein the method comprises a set of monitoring probes, wherein for each of the different identifier elements (t) of the decoding elements the set of monitoring probes comprises at least one monitoring probe with the same identifier elements (t).

14. The method according to any of claims 11 to 13, wherein the nucleic acid of the monitoring probe is attached to particle selected from a magnetic bead particle, a streptavidin coated particle, a biotin coated particle, and/or any combination thereof.

15. The method according to any of claims 11 to 14, wherein monitoring probe is designed to produce a fluorescent signal substantially equivalent to the signal intensity obtained for the target biological analytes.

16. A detection mix comprising the monitoring probe according to any of claims 1 to 10, further comprising at least one element selected from a decoy element consisting either of a magnetic bead particle, a streptavidin coated particle, a biotin coated particle, and/or any combination thereof; optionally bound to a nucleic acid oligonucleotide, which does not correspond to the identifier element (t); a decoding element comprising an identifier element (t) comprising a nucleotide sequence which is essentially complementary to at least a section of the unique set identifier sequence of the identifier element (T) of the corresponding binding element, and a translator element (c) comprising a nucleotide sequence allowing a specific hybridization of a signal oligonucleotide; a binding element which is essentially complementary to at least a section of an analyte nucleotide sequence to be detected and an identifier element (T) comprising a nucleotide sequence which is unique to the analyte nucleotide sequence; and a signal oligonucleotide comprising a translator connector element (C) comprising a nucleotide sequence which is essentially complementary to at least a section of the nucleotide sequence of a translator element (c) comprised in a decoding oligonucleotide, and a signal element facilitating a signal which is specific for the polymorphic analyte; as well as any combination thereof.

17. A method to monitor a in situ hybridization multiplex reaction with the detection mix according to claim 16,
wherein the method comprises at least the steps of:
1. contacting the analyte nucleotide sequence with at least one detecting nucleic acid sequence in order to conduct an in-situ hybridization; and
2. adding the detection mix before, during and/or after adding the detecting nucleic acid sequence in step 1.

18. The method according to claim 17,
wherein the in-situ hybridization multiplex reaction is characterized by comprising adding a:
a.) a binding element which is essentially complementary to at least a section of an analyte nucleotide sequence to be detected and an identifier element (T) comprising a nucleotide sequence which is unique to the analyte nucleotide sequence; and/or
b.) a decoding element comprising an identifier element (t) comprising a nucleotide sequence which is essentially complementary to at least a section of the unique set identifier sequence of the identifier element (T) of the corresponding binding element, and a translator element (c) comprising a nucleotide sequence allowing a specific hybridization of a signal oligonucleotide; and/or
c.) a signal oligonucleotide comprising a translator connector element (C) comprising a nucleotide sequence which is essentially complementary to at least a section of the nucleotide sequence of a translator element (c) comprised in a decoding oligonucleotide, and a signal element facilitating a signal which is specific for the polymorphic analyte; and
wherein the method comprises the detection mix according to claim 16; and
wherein the method comprises at least the steps of:
1. contacting the binding element with the analyte nucleotide sequence;
2. contacting the binding element with the decoding element; and
3. contacting the signal oligonucleotide with the decoding element; and
wherein the detection mix is added before, during and/or after adding the decoding element in step 2.

19. A method, wherein the monitoring probe according to any of claims 1 to 10, or the detection mix according to claim 16, can be detected by a signal element with a fluorophore different from the fluorophore used in the detection of the target nucleic acid sequence that permits normalizing the signal of the immobilized monitoring probe.

20. The method according to claim 19, wherein a first signal element with a fluorophore allows for measurement of hybridization efficiency of the decoder and readout probe labelled with a second signal element with a fluorophore by measuring the ratio between the first and the second fluorophore.

21. The method according to any of claim 19 or 20, wherein the first signal element with a fluorophore enables the detection of general nonspecific hybridization by identifying cycles in which the particle is labelled with a second signal element with a fluorophore that is inconsistent with the decoder element.

22. A method for generating a detection mix according to claim 16.

23. A method for controlling the density of the monitoring probes according to any of claims 1 to 10 on a slide surface by attaching azide or alkyne partner to a poly-lysine before coating it.

24. The method for controlling the density of monitoring probes according to claim 23, wherein unreactive azide or alkyne is removed, followed by a reaction step with detection mix according to claim 16.

25. A method, wherein the monitoring probes according to any of claims 1 to 10, or the detection mix according to claim 16, are placed underneath a tissue in order to assess the efficacy of a tissue sample preparation process comprising in situ oligonucleotides which penetrate through the tissue and hybridize to their intended targets.

26. A method to monitor temperature ranges of an in-situ environment in which monitoring probes according to any of claims 1 to 10 or the detection mix according to claim 16, have different delta G values corresponding to different melting points of the secondary structure in order to monitor temperature fluctuations that occur during the hybridization process.

27. The method according to claim 26, wherein the delta G value of the monitoring probes is adjusted by the sequence length or sequence composition.

28. The method according to any of claim 26 or 27, wherein the delta G value of the monitoring probes is adjusted by using modified bases which have higher stability than standard bases.

29. The method according to any of claims 26 to 28, wherein temperature fluctuation can be read-out by specific change in the fluorescence and/or colour.

30. A set of monitoring probes according to any of claims 1 to 10, or the detection mix according to claim 15, adapted to any of the methods according to any of claims 25 to 29.

31. A kit to enable optimizing in situ hybridization methods comprising slides containing immobilized monitoring probes according to any of claims 1 to 10, or the detection mix according to claim 16, and/or binding sites for monitoring probes according to any of claims 1 to 10

32. The kit according to claim 31 for monitoring a tissue sample preparation.

33. A control slide for quality control comprising a set of immobilized monitoring probes according to any of claims 1 to 9, or the detection mix according to claim 16 and/or binding sites for monitoring probes according to any of claims 1 to 10.

34. The control slide according to claim 33, for assessing in-situ hybridization instrument installations.


SEQUENCE LISTING (57 sequences)

SEQ ID NO: 1 - DNA, 20 bp, synthetic construct
  source 1..20: mol_type = other DNA
tagacagatgtggttagtct

SEQ ID NO: 2 - DNA, 23 bp, synthetic construct
  source 1..23: mol_type = other DNA
tagacctaatcttacttataacg

SEQ ID NO: 3 - DNA, 19 bp, synthetic construct
  source 1..19: mol_type = other DNA
tagacgaaggatcggaaag

SEQ ID NO: 4 - DNA, 21 bp, synthetic construct
  source 1..21: mol_type = other DNA
tagacagaatcaaaagtgaac

SEQ ID NO: 5 - DNA, 20 bp, synthetic construct
  source 1..20: mol_type = other DNA
tagacatctaggcactttac

SEQ ID NO: 6 - DNA, 20 bp, synthetic construct
  source 1..20: mol_type = other DNA
tagacaagcttctattagct

SEQ ID NO: 7 - DNA, 18 bp, synthetic construct
  source 1..18: mol_type = other DNA
tagacgttttcggcattg

SEQ ID NO: 8 - DNA, 20 bp, synthetic construct
  source 1..20: mol_type = other DNA
tagactgctataagagtccc

SEQ ID NO: 9 - DNA, 19 bp, synthetic construct
  source 1..19: mol_type = other DNA
tagacgcttactcgaagac

SEQ ID NO: 10 - DNA, 19 bp, synthetic construct
  source 1..19: mol_type = other DNA
tagacgcaaacatctttgg

SEQ ID NO: 11 - DNA, 23 bp, synthetic construct
  source 1..23: mol_type = other DNA
tagacaatactagaaagtatctg

SEQ ID NO: 12 - DNA, 22 bp, synthetic construct
  source 1..22: mol_type = other DNA
tagactactgatacaagtagat

SEQ ID NO: 13 - DNA, 20 bp, synthetic construct
  source 1..20: mol_type = other DNA
tagacaacctaaagttgcta

SEQ ID NO: 14 - DNA, 23 bp, synthetic construct
  source 1..23: mol_type = other DNA
tagaccttaagtcttaatgaaag

SEQ ID NO: 15 - DNA, 21 bp, synthetic construct
  source 1..21: mol_type = other DNA
tagacgaaacgtatattttcc

SEQ ID NO: 16 - DNA, 20 bp, synthetic construct
  source 1..20: mol_type = other DNA
tagacccgtataggatacag

SEQ ID NO: 17 - DNA, 20 bp, synthetic construct
  source 1..20: mol_type = other DNA
tagaccatattcgtaacacg

SEQ ID NO: 18 - DNA, 21 bp, synthetic construct
  source 1..21: mol_type = other DNA
tagacttgctactagtaagtg

SEQ ID NO: 19 - DNA, 22 bp, synthetic construct
  source 1..22: mol_type = other DNA
tagacccatagcttatttaagc

SEQ ID NO: 20 - DNA, 31 bp, synthetic construct
  source 1..31: mol_type = other DNA
accttaagaatggaatagactaaccacatct

SEQ ID NO: 21 - DNA, 31 bp, synthetic construct
  source 1..31: mol_type = other DNA
ggctaatttattacctagactaaccacatct

SEQ ID NO: 22 - DNA, 34 bp, synthetic construct
  source 1..34: mol_type = other DNA
accttaagaatggaatcgttataagtaagattag

SEQ ID NO: 23 - DNA, 34 bp, synthetic construct
  source 1..34: mol_type = other DNA
ggctaatttattacctcgttataagtaagattag

SEQ ID NO: 24 - DNA, 30 bp, synthetic construct
  source 1..30: mol_type = other DNA
accttaagaatggaatctttccgatccttc

SEQ ID NO: 25 - DNA, 30 bp, synthetic construct
  source 1..30: mol_type = other DNA
ggctaatttattacctctttccgatccttc

SEQ ID NO: 26 - DNA, 32 bp, synthetic construct
  source 1..32: mol_type = other DNA
accttaagaatggaatgttcacttttgattct

SEQ ID NO: 27 - DNA, 32 bp, synthetic construct
  source 1..32: mol_type = other DNA
ggctaatttattacctgttcacttttgattct

SEQ ID NO: 28 - DNA, 31 bp, synthetic construct
  source 1..31: mol_type = other DNA
accttaagaatggaatgtaaagtgcctagat

SEQ ID NO: 29 - DNA, 31 bp, synthetic construct
  source 1..31: mol_type = other DNA
ggctaatttattacctgtaaagtgcctagat

SEQ ID NO: 30 - DNA, 31 bp, synthetic construct
  source 1..31: mol_type = other DNA
accttaagaatggaatagctaatagaagctt

SEQ ID NO: 31 - DNA, 31 bp, synthetic construct
  source 1..31: mol_type = other DNA
ggctaatttattacctagctaatagaagctt

SEQ ID NO: 32 - DNA, 29 bp, synthetic construct
  source 1..29: mol_type = other DNA
accttaagaatggaatcaatgccgaaaac

SEQ ID NO: 33 - DNA, 29 bp, synthetic construct
  source 1..29: mol_type = other DNA
ggctaatttattacctcaatgccgaaaac

SEQ ID NO: 34 - DNA, 31 bp, synthetic construct
  source 1..31: mol_type = other DNA
accttaagaatggaatgggactcttatagca

SEQ ID NO: 35 - DNA, 31 bp, synthetic construct
  source 1..31: mol_type = other DNA
ggctaatttattacctgggactcttatagca

SEQ ID NO: 36 - DNA, 30 bp, synthetic construct
  source 1..30: mol_type = other DNA
accttaagaatggaatgtcttcgagtaagc

SEQ ID NO: 37 - DNA, 30 bp, synthetic construct
  source 1..30: mol_type = other DNA
ggctaatttattacctgtcttcgagtaagc

SEQ ID NO: 38 - DNA, 30 bp, synthetic construct
  source 1..30: mol_type = other DNA
accttaagaatggaatccaaagatgtttgc

SEQ ID NO: 39 - DNA, 30 bp, synthetic construct
  source 1..30: mol_type = other DNA
ggctaatttattacctccaaagatgtttgc

SEQ ID NO: 40 - DNA, 34 bp, synthetic construct
  source 1..34: mol_type = other DNA
accttaagaatggaatcagatactttctagtatt

SEQ ID NO: 41 - DNA, 34 bp, synthetic construct
  source 1..34: mol_type = other DNA
ggctaatttattacctcagatactttctagtatt

SEQ ID NO: 42 - DNA, 33 bp, synthetic construct
  source 1..33: mol_type = other DNA
accttaagaatggaatatctacttgtatcagta

SEQ ID NO: 43 - DNA, 33 bp, synthetic construct
  source 1..33: mol_type = other DNA
ggctaatttattacctatctacttgtatcagta

SEQ ID NO: 44 - DNA, 31 bp, synthetic construct
  source 1..31: mol_type = other DNA
accttaagaatggaattagcaactttaggtt

SEQ ID NO: 45 - DNA, 31 bp, synthetic construct
  source 1..31: mol_type = other DNA
ggctaatttattaccttagcaactttaggtt

SEQ ID NO: 46 - DNA, 34 bp, synthetic construct
  source 1..34: mol_type = other DNA
accttaagaatggaatctttcattaagacttaag

SEQ ID NO: 47 - DNA, 34 bp, synthetic construct
  source 1..34: mol_type = other DNA
ggctaatttattacctctttcattaagacttaag

SEQ ID NO: 48 - DNA, 32 bp, synthetic construct
  source 1..32: mol_type = other DNA
accttaagaatggaatggaaaatatacgtttc

SEQ ID NO: 49 - DNA, 32 bp, synthetic construct
  source 1..32: mol_type = other DNA
ggctaatttattacctggaaaatatacgtttc

SEQ ID NO: 50 - DNA, 31 bp, synthetic construct
  source 1..31: mol_type = other DNA
accttaagaatggaatctgtatcctatacgg

SEQ ID NO: 51 - DNA, 31 bp, synthetic construct
  source 1..31: mol_type = other DNA
ggctaatttattacctctgtatcctatacgg

SEQ ID NO: 52 - DNA, 31 bp, synthetic construct
  source 1..31: mol_type = other DNA
accttaagaatggaatcgtgttacgaatatg

SEQ ID NO: 53 - DNA, 31 bp, synthetic construct
  source 1..31: mol_type = other DNA
ggctaatttattacctcgtgttacgaatatg

SEQ ID NO: 54 - DNA, 32 bp, synthetic construct
  source 1..32: mol_type = other DNA
accttaagaatggaatcacttactagtagcaa

SEQ ID NO: 55 - DNA, 32 bp, synthetic construct
  source 1..32: mol_type = other DNA
ggctaatttattacctcacttactagtagcaa

SEQ ID NO: 56 - DNA, 33 bp, synthetic construct
  source 1..33: mol_type = other DNA
accttaagaatggaatgcttaaataagctatgg

SEQ ID NO: 57 - DNA, 33 bp, synthetic construct
  source 1..33: mol_type = other DNA
ggctaatttattacctgcttaaataagctatgg